(19) United States
(12) Patent Application Publication
(10) Pub. No.: US 2024/0173333 A1
(43) Pub. Date: May 30, 2024

(54) Treating Alagille Syndrome (ALGS)

(71) Applicant: Albireo AB, Göteborg (SE)

(72) Inventors: Jan Mattsson, Kullavik (SE); Pat Horn, Boston, MA (US)

(21) Appl. No.: 18/501,402

(22) Filed: Nov. 3, 2023

Related U.S. Application Data
(60) Provisional application No. 63/544,079, filed on Oct. 13, 2023.
(60) Provisional application No. 63/454,410, filed on Mar. 24, 2023.
(60) Provisional application No. 63/439,945, filed on Jan. 19, 2023.
(60) Provisional application No. 63/422,235, filed on Nov. 3, 2022.

(51) Int. Cl. A61K 31/554; A61P 1/16; A61P 17/04

(52) U.S. Cl. CPC A61K 31/554; A61P 1/16; A61P 17/04

(57) ABSTRACT

Provided herein are methods for Alagille syndrome (ALGS) with an ileal bile acid transport (IBAT) inhibitor such as odevixibat, or a pharmaceutically acceptable salt thereof. Such methods can include reducing pruritus score, serum bile acid concentration, increasing height, normalizing weight, improving sleep, and improving liver parameters.

### CROSS-REFERENCE TO RELATED APPLICATIONS

This application claims the benefit of U.S. Provisional Application Ser. No. 63/544,079, filed Oct. 13, 2023; and of U.S. Provisional Application Ser. No. 63/454,410, filed Mar. 24, 2023, and of U.S. Provisional Application Ser. No. 63/439,945, filed Jan. 19, 2023, and of U.S. Provisional Application Ser. No. 63/422,235, filed Nov. 3, 2022. The disclosures of the prior applications are considered part of the disclosure of this application, and are incorporated in their entirety into this application.

### TECHNICAL FIELD

Provided herein are methods for treating Alagille syndrome (ALGS) with an ileal bile acid transport (IBAT) inhibitor such as odevixibat, or a pharmaceutically acceptable salt thereof. Such methods can include reducing pruritus score, bile acid (BA) concentration, increasing height, normalizing weight, improving sleep parameters, improving quality of life, improving symptoms of itching/scratching and sleep and improving hepatic biochemical parameters and xanthomas.

### BACKGROUND

The compound 1,1-dioxo-3,3-dibutyl-5-phenyl-7-methylthio-8-(N-{(R)-α-[N-((S)-1-carboxypropyl) carbamoyl]-4-hydroxybenzyl}carbamoylmethoxy)-2,3,4,5-tetrahydro-1,2,5-benzothiadiazepine (odevixibat; also known as A4250):

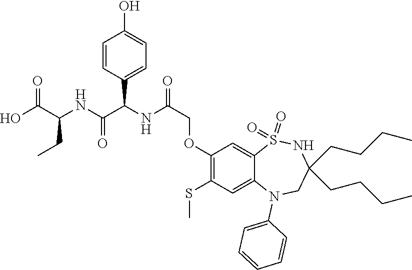 [SMILES: CCCCC1(CCCC)CN(c2ccccc2)c2cc(SC)c(OCC(=O)N[C@@H](C(=O)N[C@@H](CC)C(=O)O)c3ccc(O)cc3)cc2S(=O)(=O)N1]

is an inhibitor of the ileal bile acid transport (IBAT) mechanism. Specifically, odevixibat inhibits the natural reabsorption of bile acids from the ileum into the hepatic portal circulation. Bile acids that are not reabsorbed from the ileum are instead excreted into the feces. The overall removal of bile acids from the enterohepatic circulation leads to a decrease in the level of bile acids in serum and the liver. Odevixibat, or a pharmaceutically acceptable salt thereof, is therefore useful in the treatment of liver diseases that are associated with elevated bile acid levels, and particularly in the treatment of rare paediatric cholestatic liver diseases including Alagille syndrome (ALGS).

### SUMMARY

Provided herein are methods for treating Alagille syndrome (ALGS), in a subject in need thereof, the methods comprising orally administering to the subject a therapeutically effective amount of a pharmaceutical formulation comprising odevixibat, or a pharmaceutically acceptable salt thereof, wherein following administration of the pharmaceutical formulation, the subject exhibits a reduction from baseline in scratching score. Also provided herein are methods for treating pruritus associated with ALGS in a subject in need thereof, the methods comprising orally administering to the subject a therapeutically effective amount of a pharmaceutical formulation comprising odevixibat, or a pharmaceutically acceptable salt thereof, wherein following administration of the pharmaceutical formulation, the subject exhibits a reduction from baseline in scratching score.

Further provided herein are methods for reducing monthly scratching score in a subject having ALGS, the methods comprising orally administering to the subject a therapeutically effective amount of a pharmaceutical formulation comprising odevixibat, or a pharmaceutically acceptable salt thereof.

In some embodiments, the subject exhibits a reduction from baseline in one or more of the average AM scratching score, average PM scratching score, and average AM and PM scratching score.

In some embodiments, provided herein are methods for treating ALGS in a subject in need thereof, the methods comprising orally administering to the subject a therapeutically effective amount of a pharmaceutical formulation comprising odevixibat, or a pharmaceutically acceptable salt thereof, wherein following administration of the pharmaceutical formulation, the subject exhibits a reduction in serum bile acid concentration.

Also provided herein are methods for treating pruritus associated with ALGS in a subject in need thereof, the methods comprising orally administering to the subject a therapeutically effective amount of a pharmaceutical formulation comprising odevixibat, or a pharmaceutically acceptable salt thereof, wherein following administration of the pharmaceutical formulation, the subject exhibits a reduction in serum bile acid concentration.

Further provided herein are methods for reducing serum bile acid concentration in a subject having ALGS, the methods comprising orally administering to the subject a therapeutically effective amount of a pharmaceutical formulation comprising odevixibat, or a pharmaceutically acceptable salt thereof.

Provided herein are methods for treating ALGS in a subject in need thereof, the methods comprising orally administering to the subject a therapeutically effective amount of a pharmaceutical formulation comprising odevixibat, or a pharmaceutically acceptable salt thereof, wherein following administration of the pharmaceutical formulation for at least 4 weeks, the subject exhibits a change from baseline in serum bile acid concentration.

Also provided herein are methods for treating pruritus associated with ALGS in a subject in need thereof, the methods comprising orally administering to the subject a therapeutically effective amount of a pharmaceutical formulation comprising odevixibat, or a pharmaceutically acceptable salt thereof, wherein following administration of the pharmaceutical formulation for at least 4 weeks, the subject exhibits a serum bile acid concentration of less than 70 μmol/L.

Further provided herein are methods for treating ALGS in a subject in need thereof, the methods comprising orally administering to the subject a therapeutically effective amount of a pharmaceutical formulation comprising odevixibat, or a pharmaceutically acceptable salt thereof, wherein following administration of the pharmaceutical formulation for at least 24 weeks, the subject exhibits a reduction from baseline in serum bile acid concentrations.

In some embodiments, provided herein are methods for treating ALGS in a subject in need thereof, the methods comprising orally administering to the subject a therapeutically effective amount of a pharmaceutical formulation comprising odevixibat, or a pharmaceutically acceptable salt thereof, wherein following administration of the pharmaceutical formulation for at least 24 weeks, the subject exhibits a reduction in serum bile acid concentration of at least 50% relative to baseline.

Also provided herein are methods for reducing serum bile acid concentrations relative to baseline in a subject having ALGS, the method comprising orally administering to the subject a therapeutically effective amount of a pharmaceutical formulation comprising odevixibat, or a pharmaceutically acceptable salt thereof, for about 4 to about 24 weeks. For example, about 4 to about 8 weeks, about 8 to about 12 weeks, about 12 to about 16 weeks, about 16 to about 20 weeks, or about 20 to about 24 weeks.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 provides a schematic of the study design and key inclusion criteria for the ALGS ASSERT placebo-controlled trial.

FIG. 2 illustrates the disposition of patients in the double-blind, randomized, placebo-controlled ALGS ASSERT study. There were 52 patients who enrolled in the study: 17 were given a placebo and 35 were given odevixibat. All 52 patients (100%) completed the study and 50 patients electively enrolled in the open-label extension study.

FIG. 3 provides the baseline demographics and disease characteristics for patients in the placebo-controlled ALGS ASSERT trial.

FIG. 4 is a bar graph showing the least squares (LS) mean change from baseline in scratching score to Weeks 21-24 of treatment with odevixibat versus placebo.

FIG. 5 is a line graph showing the least squares (LS) mean (95% confidence interval (CI)) change from baseline in scratching score to Weeks 21-24 of treatment with odevixibat versus placebo.

FIG. 6 is a bar graph showing the least squares (LS) mean change from baseline in bile acids (μmol/L) to the average of Weeks 20 and 24 of treatment with odevixibat versus placebo.

FIG. 7 is a line graph showing the least squares (LS) mean (95% confidence interval (CI)) change in bile acids (μmol/L) over time from baseline to the average of Weeks 20 and 24 of treatment with odevixibat versus placebo.

FIG. 8A is a bar graph showing the percentage of patients with a pruritus response (a pruritus response is defined as a ≥1-point reduction in the monthly scratching score from baseline) at weeks 9-12 and weeks 21-24 of treatment with odevixibat versus placebo.

FIG. 8B is a table showing the number of patients with a pruritus response (a pruritus response is defined as a ≥1.5-point reduction in the monthly scratching score from baseline) at weeks 9-12 and weeks 21-24 of treatment with odevixibat versus placebo.

FIG. 8C is a bar graph showing the percentage of patients with a pruritus response (a pruritus response is defined as a ≥1.5-point reduction in the monthly scratching score from baseline) at weeks 9-12 and weeks 21-24 of treatment with odevixibat versus placebo.

FIG. 9 is a line graph showing the least squares (LS) mean (95% confidence interval (CI)) change in percentage of days of patients sleeping with the caregiver from baseline to Weeks 21-24 of treatment with odevixibat versus placebo.

FIG. 10 provides the least squares (LS) mean change in all sleep parameters measured by the ObsRO from baseline to Weeks 21-24 of treatment with odevixibat versus placebo.

FIG. 11A is a bar graph showing the global impression of change (GIC) for scratching using the percentage of clinician responses at week 24 of treatment with odevixibat versus placebo.

FIG. 11B is a bar graph showing the global impression of change (GIC) for sleep using the percentage of clinician responses at week 24 of treatment with odevixibat versus placebo.

FIG. 11C is a bar graph showing the global impression of change (GIC) for scratching using the percentage of caregiver responses at week 24 of treatment with odevixibat versus placebo.

FIG. 11D is a bar graph showing the global impression of change (GIC) for sleep using the percentage of caregiver responses at week 24 of treatment with odevixibat versus placebo.

FIG. 11E is a two bar graph showing the global impression of change (GIC) for itching using the percentage of patient responses at week 24 of treatment with odevixibat versus placebo.

FIG. 11F is a bar graph showing the global impression of change (GIC) for sleep using the percentage of patient responses at week 24 of treatment with odevixibat versus placebo.

FIG. 12A is a bar graph showing the mean (SD) change from baseline to Weeks 21-24 in scratching score of patients with JAG1 mutations and NOTCH2 mutations (after treatment with odevixibat versus placebo).

FIG. 12B is a bar graph showing the change from baseline to the average of Weeks 20 and 24 in bile acids (μmol/L) (right) of patients with JAG1 mutations and NOTCH2 mutations (after treatment with odevixibat versus placebo).

FIG. 13 provides the safety summary for all patients during treatment with odevixibat versus placebo. No patients discontinued the ASSERT placebo-controlled and no deaths or treatment-emergent adverse events (TEAEs) leading to discontinuation of treatment were reported.

FIG. 14 is a modified eDISH plot showing peak total bilirubin (x baseline) and the peak alanine aminotransferase (ALT) (x baseline) for each patient from post-baseline to Weeks 21-24 of treatment with odevixibat versus placebo.

FIG. 15 provides the baseline ALT and total bilirubin data of patients and the change from baseline to Weeks 24 in ALT and total bilirubin in patients after treatment with odevixibat versus placebo.

FIG. 16A is a bar graph showing the mean change in clinician xanthoma score of patients from baseline to Weeks 12 and 24 of treatment with odevixibat versus placebo.

FIG. 16B is a line graph showing the mean change from baseline over time in serum cholesterol levels (mmol/L) patients to Weeks 24 of treatment with odevixibat versus placebo.

FIG. 17 is a table summarizing the exposure and pooled baseline and post treatment outcomes in patients with ALGS treated with odevixibat in ASSERT and ASSERT-EXT to weeks 9-12 (pruritus and sleep) and Week 12 (bile acids).

FIG. 18A is a plot showing the mean (SD) scratching score over time in a pooled population of patients with ALGS treated with odevixibat.

FIG. 18B is a plot showing the mean (SD) bile acid levels over time in a pooled population of patients with ALGS treated with odevixibat.

FIG. 18C is a plot showing the mean (SD) autotaxin levels over time in a pooled population of patients with ALGS treated with odevixibat.

FIG. 18D is a plot showing the mean (SD) C4 levels over time in a pooled population of patients with ALGS treated with odevixibat.

FIG. 19A is a plot showing serum bile acid levels before and after odevixibat treatment in a patient found to have Alagille syndrome. Vertical dashed lines indicate odevixibat start, and shading indicates the different doses of odevixibat that were administered.

FIG. 19B is a plot showing total bilirubin levels before and after odevixibat treatment in a patient found to have Alagille syndrome. Vertical dashed lines indicate odevixibat start, and shading indicates the different doses of odevixibat that were administered.

FIG. 20 is a flow diagram of key disease features throughout the life of a patient found to have Alagille syndrome and the impact of odevixibat treatment. ALT, alanine aminotransferase; AST, aspartate aminotransferase; GGT, gamma-glutamyl transferase; UDCA, ursodeoxycholic acid.

FIG. 21 is a table showing a blinded sample size re-estimation.

FIG. 22 is a line graph showing the mean (SE) scratching score over time from the ASSERT baseline to Weeks 9-12 of ASSERT-EXT of treatment with placebo-odevixibat versus odevixibat-odevixibat. Values shown for ASSERT timepoints represent all patients randomized in ASSERT (placebo, n=17; odevixibat, n=35); values shown for ASSERT-EXT timepoints represent only those patients who rolled over to ASSERT-EXT and received odevixibat prior to the data cut-off date (placebo-odevixibat, n=17; odevixibat-odevixibat, n=32). Scratching score ranges from 0-4, with higher scores indicating worse symptoms. A pruritus reduction ≥1-point from baseline was achieved in 70% of patients in the placebo-odevixibat group at weeks 9-12 of ASSERT-EXT.

FIG. 23 is a line graph showing the mean (SE) bile acid level (μmol/L) over time from the ASSERT baseline to Weeks 9-12 of ASSERT-EXT of treatment with placebo-odevixibat versus odevixibat-odevixibat. Values shown for ASSERT timepoints represent all patients enrolled in ASSERT (placebo, n=17; odevixibat, n=35); values shown for ASSERT-EXT timepoints represent only those patients who rolled over to ASSERT-EXT and received odevixibat prior to the data cut-off date (placebo-odevixibat, n=17; odevixibat-odevixibat, n=32).

FIG. 24A is a line graph showing the mean (SE) tiredness score over time from the ASSERT baseline to Weeks 9-12 of ASSERT-EXT of treatment with placebo-odevixibat versus odevixibat-odevixibat. Values shown for ASSERT timepoints represent all patients enrolled in ASSERT (placebo, n=17; odevixibat, n=35); values shown for ASSERT-EXT timepoints represent only those patients who rolled over to ASSERT-EXT and received odevixibat prior to the data cut-off date (placebo-odevixibat, n=17; odevixibat-odevixibat, n=32). Tiredness score ranges from 0-4, with higher scores indicating worse symptoms.

FIG. 24B is a table showing all sleep parameters.

FIG. 25 is a table showing a summary of treatment emergent adverse events.

FIG. 26 is an example of the observer-reported items of the PRUCISION™ instrument.

FIG. 27 is a table showing global impressions of change (GIC) over time. GIC items are assessed on a 7-point scale: 1=very much better; 2=much/moderately better; 3=a little better; 4=no change; 5=a little worse; 6=much/moderately worse; 7=very much worse. Changes based on the GIC were analyzed using a proportional odds model with treatment and baseline GIS scores as covariates considering three categories (better, no change, worse). Better includes “very much better,” “much better,” and “a little better,” and worse includes “a little worse,” “much worse,” and “very much worse.” CaGIC=Caregiver Global Impression of Change. CGIC=Clinician Global Impression of Change. PGIC=Patient Global Impression of Change.

FIG. 28 is a table showing global impressions of symptoms (GIS) over time. CaGIS=Caregiver Global Impression of Symptoms. CGIS=Clinician Global Impression of Symptoms. PGIS=Patient Global Impression of Symptoms.

FIGS. 29A and 29B are line graphs showing change from baseline in scratching scores (FIG. 29A) and bile acids (FIG. 29B) with odevixibat treatment in pooled patients in ASSERT and ASSERT-EXT studies.

FIG. 30 is a plot showing serum bile acids levels in individual patients with ALGS who received odevixibat in ASSERT and/or ASSERT-EXT.

FIG. 31 is a plot showing scratching scores from individual patients with ALGS who received odevixibat in ASSERT and/or ASSERT-EXT.

FIG. 32A is a plot showing mean change in baseline in ALT with odevixibat treatment in pooled patients in ASSERT and ASSERT-EXT studies.

FIG. 32B is a plot showing mean change in baseline in AST with odevixibat treatment in pooled patients in ASSERT and ASSERT-EXT studies.

FIG. 32C is a plot showing mean change in baseline in GGT with odevixibat treatment in pooled patients in ASSERT and ASSERT-EXT studies.

FIG. 32D is a plot showing mean change in baseline in total bilirubin with odevixibat treatment in pooled patients in ASSERT and ASSERT-EXT studies.

FIGS. 33A and 33B are bar graphs showing the percentage of patients in ASSERT with FSV insufficiency at baseline and Week 24 for patients receiving odevixibat (FIG. 33A) and placebo (FIG. 33B). ^{a}n=32 for total population assessed for vitamin D insufficiency and any vitamin insufficiency. Vitamin insufficiency was defined as low vitamin levels based on the lower limit of normal reference ranges in relevant age groups for vitamin A (0.2 mg/L), vitamin D (25-hydroxyvitamin D; 20 ng/L), and vitamin E (alpha tocopherol; 2.0 mg/L), and the upper limit of normal reference range for vitamin K (INR; 1.1). FSV, fat-soluble vitamin; INR, international normalized ratio.

FIGS. 34A and 34B are a plot (FIG. 34A) and a violin plot (FIG. 34B) showing the change from baseline in scratching score in odevixibat-treated pruritus responders during ASSERT. The violin plot depicts the distribution and relative density of data over the range of possible scratching score values; the dotted lines indicate the quartile values (quartile 1, quartile 3), and the dashed line indicates the median value.

FIGS. 35A and 35B are a plot (FIG. 35A) and a violin plot (FIG. 35B) showing the change from baseline in bile acids in odevixibat-treated pruritus responders during ASSERT. The violin plot depicts the distribution and relative density of data over the range of observed bile acid values; the dotted lines indicate the quartile values (quartile 1, quartile 3), and the dashed line indicates the median value. ^{a}Average of weeks 20 and 24.

FIG. 36 is a box-and-whisker plot showing the change from baseline in sleep parameters in odevixibat-treated pruritus responders during ASSERT. In the box-and-whisker plot, the maximum and minimum values are indicated by the whiskers, the quartile values (quartile 1, quartile 3) by the ends of the box, the median value by the horizontal line in the box, and the mean value by the “+” symbol. Plot depicts data from n=27 patients for each parameter.

FIG. 37 is a box-and-whisker plot showing the change from baseline in total and domain scores of the PedsQL in odevixibat-treated pruritus responders. PedsQL total and domain scores range from 0 to 100, with higher scores indicating better functioning. In the box-and-whisker plot, the maximum and minimum values are indicated by the whiskers, the quartile values (quartile 1, quartile 3) by the ends of the box, the median value by the horizontal line in the box, and the mean value by the “+” symbol. Graph depicts data from n=23 patients for each score. PedsQL, Pediatric Quality of Life Inventory; QoL, quality of life.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “subject,” “individual,” or “patient,” used interchangeably, refer to any animal, including mammals such as mice, rats, other rodents, rabbits, dogs, cats, swine, cattle, sheep, horses, primates, and humans. In some embodiments, the subject is a human.

The term “pediatric” as used herein refers to a subject under the age of 21 years at the time of diagnosis or treatment. The term “pediatric” can be further divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E, Textbook of Pediatrics, 15th Ed. Philadelphia: W.B. Saunders Company, 1996; Rudolph A M, et al., Rudolph's Pediatrics, 21st Ed. New York: McGraw-Hill, 2002; and Avery M D, First L R, Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

As used herein, the term “baseline” refers to information obtained prior to the first administration of the drug or intervention of interest (e.g., at the beginning of a study) or an initial known value that is used for comparison with later data. Baseline values are taken at time “zero” (i.e., before subjects in a study receive the drug or intervention of interest or placebo).

As used herein, the term “normalized” refers to age-specific values that are within a range corresponding to a healthy individual (i.e., normal or normalized values).

As used herein, the term “pharmaceutically acceptable” refers to those compounds, materials, compositions and/or dosage forms that are suitable for human pharmaceutical use and that are generally safe, non-toxic and neither biologically nor otherwise undesirable.

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

The term “crystal modification” refers to a crystalline solid phase of an organic compound. A crystal modification can be either a solvate or an ansolvate.

The term “solvate” refers to a crystalline solid phase of an organic compound, which has solvent (i.e., solvent molecules) incorporated into its crystal structure. A “hydrate” is a solvate wherein the solvent is water.

The term “sesquihydrate” refers to a hydrate containing about 1.5 moles of water associated with the crystal per mole of organic compound (i.e., a 1.5 hydrate). As used herein, a sesquihydrate includes from about 1.2 to about 1.8, for example, from about 1.3 to about 1.7, about 1.4 to about 1.6, or about 1.45 to about 1.55 moles of water associated with each mole of odevixibat in a crystal. The amount of water calculated herein excludes water adsorbed to the surface of the crystal.

The term “mixed solvate” refers to a crystalline solid phase of an organic compound, which has two or more different solvent molecules incorporated into its crystal structure. One of the at least two solvent molecules may be water.

The term “slurry” refers to a saturated solution to which an excess of solid is added, thereby forming a mixture of solid and saturated solution.

As used herein, the term “void volumes” refers to channels, layers or other more or less isolated voids in the crystal structure.

The crystallinity of a crystalline sample of odevixibat may be measured e.g. by X-Ray Powder Diffraction (XRPD) methods or by Differential Scanning calorimetry (DSC) methods, such as the method disclosed in the experimental section. When reference is made herein to a crystalline compound, the crystallinity as measured by DSC methods is greater than about 70%, such as greater than about 80%, particularly greater than about 90%, more particularly greater than about 95%. In some embodiments, the degree of crystallinity as measured by DSC methods is greater than about 98%. In some embodiments, the degree of crystallinity as measured by DSC methods is greater than about 99%. The % crystallinity refers to the percentage by weight of the total sample mass which is crystalline.

### Methods of Treating ALGS

Alagille syndrome (ALGS) is a rare, multisystem disorder with a wide variety of clinical manifestations affecting the liver, heart, skeleton, eyes, central nervous system, kidneys, and facial features. It is an autosomal dominantly-inherited disorder caused by defects in components of the NOTCH signaling pathway, most commonly due to mutations in JAG1, in about 90% of the patients. A small number of patients with ALGS have mutations in the gene for the NOTCH2 receptor. Approximately 60% of the cases represent de novo mutations. The majority of patients present early, often within the first 3 months of life, with jaundice or cardiac symptoms.

One manifestation of ALGS is pruritus, with up to 88% of patients presenting with pruritus and up to 45% having severe pruritus, which often results in a severely diminished quality of life. In some cases, ALGS leads to cirrhosis and liver failure. Current therapies include Partial External Biliary Diversion (PEBD) and liver transplantation, however, these options can carry substantial risk of post-surgical complications, as well as psychological and social issues.

The IBAT, also called the apical sodium-dependent bile acid transporter (SLC10A2), is located on the luminal surface of enterocytes in the terminal ileum; this transporter mediates resorption of conjugated bile acids for recirculation back to the liver. Inhibition of IBAT disrupts the enterohepatic circulation and leads to fecal elimination of bile acids similar to surgical interruption of the enterohepatic circulation.

Odevixibat is an orally administered, potent, luminally restricted, selective IBAT inhibitor in development to treat cholestatic liver diseases. By inhibiting IBAT with high selectivity and potency, odevixibat can reduce the elevations in systemic bile acids that result from cholestasis and decrease pruritus in patients with ALGS. The rationale for using odevixibat is to decrease serum bile acid levels, and to reduce the major morbidity of pruritus, improving the health and wellbeing of patients affected with ALGS. By reducing the elevations in systemic bile acids, odevixibat also can improve liver function and modify the progression of liver damage in patients with ALGS.

Provided herein are methods for treating ALGS in a subject in need thereof, the method comprising orally administering to the subject a therapeutically effective amount of a pharmaceutical formulation comprising odevixibat, or a pharmaceutically acceptable salt thereof. Also provided herein are methods for treating pruritus associated with ALGS in a subject in need thereof, the method comprising orally administering to the subject a therapeutically effective amount of a pharmaceutical formulation comprising odevixibat, or a pharmaceutically acceptable salt thereof. In some embodiments, provided herein are methods for treating cholestasis associated with ALGS in a subject in need thereof, the method comprising orally administering to the subject a therapeutically effective amount of a pharmaceutical formulation comprising odevixibat, or a pharmaceutically acceptable salt thereof.

Also provided herein is a pharmaceutical formulation comprising odevixibat, or a pharmaceutically acceptable salt thereof, for use in treating ALGS, and for use in treating pruritus associated with ALGS. In some embodiments, also provided herein is a pharmaceutical formulation comprising odevixibat, or a pharmaceutically acceptable salt thereof, for use in treating ALGS, and for use in treating cholestasis associated with ALGS.

Also provided herein is the use of a pharmaceutical formulation comprising odevixibat, or a pharmaceutically acceptable salt thereof, for use in the manufacture of a medicament for the treatment of ALGS, and for the treatment of pruritus associated with ALGS. In some embodiments, provided herein is the use of a pharmaceutical formulation comprising odevixibat, or a pharmaceutically acceptable salt thereof, in the treatment of cholestasis associated with ALGS.

Odevixibat, as referred to herein, includes solvates and hydrates thereof. For example, odevixibat can be present as a hydrate (e.g., a sesquihydrate).

In some embodiments, for patients with ALGS, reducing pruritus is an important therapeutic goal. However, few instruments are available that adequately measure pruritus in patients, such as pediatric patients with ALGS. The pruritus score (also referred to herein as a “scratching score”) disclosed herein can be measured according to the PRUCISION™ patient-reported outcome (PRO) and observer-reported outcome (ObsRO) instruments to estimate a threshold for clinically meaningful change in pruritus score. These instruments can be used to demonstrate the change from baseline in pruritus score and calculate the percentage of patients who achieve a clinically meaningful response. For a description of PRUCISION™ instruments see, e.g., Gwaltney et al., Adv. Ther. (2022), 39:5105-5125, which is incorporated by reference herein in its entirety.

In some embodiments, following administration of odevixibat, or a pharmaceutically acceptable salt thereof, the subject exhibits a reduction in mean monthly pruritus score.

In some embodiments, the reduction in mean monthly pruritus score (i.e., scratching score) is at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, at least 3.4, at least 3.5, at least 3.6, at least 3.7, at least 3.8, or at least 3.9. In some embodiments, the reduction in mean monthly pruritus score is about 0.3 to about 4.0. For example, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.5 to about 3.5, about 0.5 to about 3.0, about 0.5 to about 2.5, about 0.5 to about 2.0, about 0.5 to about 1.5, about 0.5 to about 1.0, about 1.0 to about 4.0 about 1.0 to about 3.5, about 1.0 to about 3.0, about 1.0 to about 2.5, about 1.0 to about 2.0, about 1.0 to about 1.5, about 1.5 to about 4.0, about 1.5 to about 3.5, about 1.5 to about 3.0, about 1.5 to about 2.5, about 1.5 to about 2.0, about 2.0 to about 1.0, about 2.0 to about 3.5, about 2.0 to about 3.0, about 2.0 to about 2.5, about 2.5 to about 4.0 about 2.5 to about 3.5, about 2.5 to about 3.0, about 3.0 to about 4.0, about 3.0 to about 3.5, or about 3.5 to about 4.0. In some embodiments, the reduction in mean monthly pruritus score is about 0.5 to about 2.0 (e.g., about 0.5 to about 1.5; about 0.5 to about 1.2; about 0.8 to about 1.4; about 0.9 to about 1.2; about 1.2 to about 2.0; about 1.2 to about 1.5; about 1.2 to about 1.8; about 1.4 to about 2.0; about 1.6 to about 2.0; about 1.5 to about 2.0; about 1.3 to about 1.6; or about 1.4 to about 1.8). In some embodiments, the reduction in mean monthly pruritus score is about 2.0. In some embodiments, the reduction in mean monthly pruritus score is about 1.6.

In some embodiments, the reduction in mean monthly pruritus score occurs after about 1 week to about 72 weeks of administration of odevixibat, or a pharmaceutically acceptable salt thereof. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of odevixibat, or a pharmaceutically acceptable salt thereof. In some embodiments, the reduction in mean monthly pruritus score occurs after about 5 weeks to about 8 weeks, about 9 to about 12 weeks, about 21 to about 24 weeks of administration of odevixibat, or a pharmaceutically acceptable salt thereof.

In some embodiments, the reduction in mean monthly pruritus score occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of odevixibat, or a pharmaceutically acceptable salt thereof.

In some embodiments, the reduction in mean monthly pruritus score occurs following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the reduction in mean monthly pruritus score occurs following administration of odevixibat, or a pharmaceutically acceptable salt thereof, for at least 4 weeks, at least 8 weeks, at least 12 weeks, at least 16 weeks, at least 20 weeks, or at least 24 weeks. In some embodiments, the reduction in mean monthly pruritus score occurs following 4 weeks of administration. In some embodiments, the reduction in mean monthly pruritus score occurs following 24 weeks of administration. In some embodiments, the reduction in mean monthly pruritus score occurs following 48 weeks of administration. In some embodiments, the reduction in mean monthly pruritus score occurs following 72 weeks of administration.

In some embodiments, the reduction in mean monthly pruritus score is about 0.3 to about 2.0 following 24 weeks of administration of odevixibat, or a pharmaceutically acceptable salt thereof. In some embodiments, the reduction in mean monthly pruritus score is about 0.5 to about 1.5 following 24 weeks of administration of odevixibat, or a pharmaceutically acceptable salt thereof. For example, the reduction in mean monthly pruritus score is about 0.9 to about 1.3 following 24 weeks of administration of odevixibat, or a pharmaceutically acceptable salt thereof. In some embodiments, the reduction in mean monthly pruritus score is about 1.1 following 24 weeks of administration of odevixibat, or a pharmaceutically acceptable salt thereof.

In some embodiments, the reduction in mean monthly pruritus score is about 1.2 to about 2.0 following 48 weeks of administration of odevixibat, or a pharmaceutically acceptable salt thereof. For example, the reduction in mean monthly pruritus score is about 1.4 to about 1.8 following 48 weeks of administration of odevixibat, or a pharmaceutically acceptable salt thereof. In some embodiments, the reduction in mean monthly pruritus score is about 1.6 following 48 weeks of administration of odevixibat, or a pharmaceutically acceptable salt thereof.

In some embodiments, the mean monthly pruritus score is normalized following administration of odevixibat, or a pharmaceutically acceptable salt thereof. In some embodiments, the mean monthly pruritus score is normalized following 48 weeks of administration of odevixibat, or a pharmaceutically acceptable salt thereof.

In some embodiments, following administration of odevixibat, or a pharmaceutically acceptable salt thereof, the subject exhibits a reduction in pruritus score relative to baseline. In some embodiments, the reduction in pruritus score (i.e., scratching score) relative to baseline is at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, at least 3.4, at least 3.5, at least 3.6, at least 3.7, at least 3.8, or at least 3.9. In some embodiments, the reduction in pruritus score relative to baseline is about 0.3 to about 4.0. For example, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.5 to about 3.5, about 0.5 to about 3.0, about 0.5 to about 2.5, about 0.5 to about 2.0, about 0.5 to about 1.5, about 0.5 to about 1.0, about 1.0 to about 4.0 about 1.0 to about 3.5, about 1.0 to about 3.0, about 1.0 to about 2.5, about 1.0 to about 2.0, about 1.0 to about 1.5, about 1.5 to about 4.0, about 1.5 to about 3.5, about 1.5 to about 3.0, about 1.5 to about 2.5, about 1.5 to about 2.0, about 2.0 to about 1.0, about 2.0 to about 3.5, about 2.0 to about 3.0, about 2.0 to about 2.5, about 2.5 to about 4.0 about 2.5 to about 3.5, about 2.5 to about 3.0, about 3.0 to about 4.0, about 3.0 to about 3.5, or about 3.5 to about 4.0. In some embodiments, the reduction in pruritus score relative to baseline is about 0.5 to about 2.0 (e.g., about 0.5 to about 1.5; about 0.5 to about 1.2; about 0.8 to about 1.4; about 0.9 to about 1.2; about 1.2 to about 2.0; about 1.2 to about 1.5; about 1.2 to about 1.8; about 1.4 to about 2.0; about 1.6 to about 2.0; about 1.5 to about 2.0; about 1.3 to about 1.6; or about 1.4 to about 1.8). In some embodiments, the reduction in pruritus score relative to baseline is about 2.0. In some embodiments, the reduction in pruritus score relative to baseline is about 1.6.

In some embodiments, the subject exhibits a scratching score of 2.5 or more prior to the first administration of a pharmaceutical formulation comprising odevixibat, or a pharmaceutically acceptable salt thereof. For example, the subject can exhibit a scratching score of about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 prior to the first administration of a pharmaceutical formulation comprising odevixibat, or a pharmaceutically acceptable salt thereof.

In some embodiments, the subject exhibits an average scratching score of about 2.5 to about 4.0 prior to the first administration of a pharmaceutical formulation comprising odevixibat, or a pharmaceutically acceptable salt thereof. For example, the subject can exhibit an average scratching score of about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 prior to the first administration of a pharmaceutical formulation comprising odevixibat, or a pharmaceutically acceptable salt thereof. In some embodiments, the subject exhibits an average scratching score of about 2.8 prior to the first administration of a pharmaceutical formulation comprising odevixibat, or a pharmaceutically acceptable salt thereof.

In some embodiments, the reduction in pruritus score relative to baseline occurs after about 1 week to about 72 weeks of administration of odevixibat, or a pharmaceutically acceptable salt thereof. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of odevixibat, or a pharmaceutically acceptable salt thereof. In some embodiments, the reduction in pruritus score relative to baseline occurs after about 5 weeks to about 8 weeks, about 9 to about 12 weeks, about 13 to about 16 weeks, about 17 to about 20 weeks, about 21 to about 24 weeks of administration of odevixibat, or a pharmaceutically acceptable salt thereof.

In some embodiments, the reduction in pruritus score relative to baseline occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of odevixibat, or a pharmaceutically acceptable salt thereof.

In some embodiments, the reduction in pruritus score relative to baseline occurs following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the reduction in pruritus score relative to baseline occurs following administration of odevixibat, or a pharmaceutically acceptable salt thereof, for at least 4 weeks, at least 8 weeks, at least 12 weeks, at least 16 weeks, at least 20 weeks, or at least 24 weeks. In some embodiments, the reduction in pruritus score relative to baseline occurs following 1 week of administration. In some embodiments, the reduction in pruritus score relative to baseline occurs following 4 weeks of administration. In some embodiments, the reduction in pruritus score relative to baseline occurs following 24 weeks of administration. In some embodiments, the reduction in pruritus score relative to baseline occurs following 48 weeks of administration. In some embodiments, the reduction in pruritus score relative to baseline occurs following 72 weeks of administration.

In some embodiments, the reduction in pruritus score relative to baseline is about 0.3 to about 2.0 following 24 weeks of administration of odevixibat, or a pharmaceutically acceptable salt thereof. In some embodiments, the reduction in pruritus score relative to baseline is about 0.5 to about 1.5 following 24 weeks of administration of odevixibat, or a pharmaceutically acceptable salt thereof. For example, the reduction in pruritus score relative to baseline is about 0.9 to about 1.3 following 24 weeks of administration of odevixibat, or a pharmaceutically acceptable salt thereof. In some embodiments, the reduction in pruritus score relative to baseline is about 1.1 following 24 weeks of administration of odevixibat, or a pharmaceutically acceptable salt thereof. In some embodiments, the reduction in pruritus score relative to baseline is about 2.0 following about 24 weeks of administration of odevixibat, or a pharmaceutically acceptable salt thereof.

In some embodiments, the reduction in pruritus score relative to baseline is about 1.2 to about 2.0 following 48 weeks of administration of odevixibat, or a pharmaceutically acceptable salt thereof. For example, the reduction in pruritus score relative to baseline is about 1.4 to about 1.8 following 48 weeks of administration of odevixibat, or a pharmaceutically acceptable salt thereof. In some embodiments, the reduction in pruritus score relative to baseline is about 1.6 following 48 weeks of administration of odevixibat, or a pharmaceutically acceptable salt thereof.

In some embodiments, the reduction is pruritus score (i.e., scratching score) relative to baseline is a reduction in: the average AM scratching score, the average PM scratching score, or average AM and PM scratching score. In some embodiments, following administration of odevixibat, or a pharmaceutically acceptable salt thereof, the subject exhibits a reduction in the average AM and PM scratching score relative to baseline. In some embodiments, the average AM and PM scratching score is the worst scratching score as measured by the ObsRO instrument. In some embodiments, the average AM and PM scratching score is the worst scratching score as measured by the ObsRO AM and PM caregiver reported instrument.

In some embodiments, the reduction in the average AM scratching score, the average PM scratching score, or average AM and PM scratching score relative to baseline is at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, at least 3.4, at least 3.5, at least 3.6, at least 3.7, at least 3.8, or at least 3.9. In some embodiments, the reduction in the average AM scratching score, the average PM scratching score, or average AM and PM scratching score relative to baseline is about 0.3 to about 4.0. For example, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.5 to about 3.5, about 0.5 to about 3.0, about 0.5 to about 2.5, about 0.5 to about 2.0, about 0.5 to about 1.5, about 0.5 to about 1.0, about 1.0 to about 4.0 about 1.0 to about 3.5, about 1.0 to about 3.0, about 1.0 to about 2.5, about 1.0 to about 2.0, about 1.0 to about 1.5, about 1.5 to about 4.0, about 1.5 to about 3.5, about 1.5 to about 3.0, about 1.5 to about 2.5, about 1.5 to about 2.0, about 2.0 to about 1.0, about 2.0 to about 3.5, about 2.0 to about 3.0, about 2.0 to about 2.5, about 2.5 to about 4.0 about 2.5 to about 3.5, about 2.5 to about 3.0, about 3.0 to about 4.0, about 3.0 to about 3.5, or about 3.5 to about 4.0. In some embodiments, the reduction in the average AM scratching score, the average PM scratching score, or average AM and PM scratching score relative to baseline is about 0.5 to about 2.0 (e.g., about 0.5 to about 1.5; about 0.5 to about 1.2; about 0.8 to about 1.4; about 0.9 to about 1.2; about 1.2 to about 2.0; about 1.2 to about 1.5; about 1.2 to about 1.8; about 1.4 to about 2.0; about 1.6 to about 2.0; about 1.5 to about 2.0; about 1.3 to about 1.6; or about 1.4 to about 1.8). In some embodiments, the reduction in the average AM scratching score, the average PM scratching score, or average AM and PM scratching score relative to baseline is about 1.1. In some embodiments, reduction in the average AM scratching score, the average PM scratching score, or average AM and PM scratching score relative to baseline is about 1.6. In some embodiments, the reduction in the average AM scratching score, the average PM scratching score, or average AM and PM scratching score relative to baseline is about 2.0.

In some embodiments, reduction in the average AM scratching score, the average PM scratching score, or average AM and PM scratching score relative to baseline occurs after about 1 week to about 72 weeks of administration of odevixibat, or a pharmaceutically acceptable salt thereof. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 1 week to about 72 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of odevixibat, or a pharmaceutically acceptable salt thereof. In some embodiments, the reduction in the average AM scratching score, the average PM scratching score, or average AM and PM scratching score relative to baseline occurs after about 5 weeks to about 8 weeks, about 9 to about 12 weeks, about 21 to about 24 weeks of administration of odevixibat, or a pharmaceutically acceptable salt thereof.

In some embodiments, the reduction in the average AM scratching score, the average PM scratching score, or average AM and PM scratching score relative to baseline occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of odevixibat, or a pharmaceutically acceptable salt thereof.

In some embodiments, the reduction in the average AM scratching score, the average PM scratching score, or average AM and PM scratching score relative to baseline occurs following administration of odevixibat, or a pharmaceutically acceptable salt thereof, for at least 1 week, at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, reduction in the average AM scratching score, the average PM scratching score, or average AM and PM scratching score relative to baseline occurs following administration of odevixibat, or a pharmaceutically acceptable salt thereof, for at least 1 week, at least 4 weeks, at least 8 weeks, at least 12 weeks, at least 16 weeks, at least 20 weeks, or at least 24 weeks. In some embodiments, the subject exhibits a reduction in mean serum bile acid concentration.

In some embodiments, the reduction from baseline in mean serum bile acid concentration is at least 25 μmol/L, at least 50 μmol/L, at least 75 μmol/L, at least 100 μmol/L, at least 125 μmol/L, at least 150 μmol/L, at least 175 μmol/L, at least 200 μmol/L, at least 300 μmol/L, at least 400 μmol/L, at least 500 μmol/L, or at least 600 μmol/L relative to baseline. For example, the reduction in mean serum bile acid concentration is about 25 μmol/L to about 200 μmol/L relative to baseline (e.g., about 25 μmol/L to about 50 μmol/L; about 25 μmol/L to about 50 μmol/L; about 25 μmol/L to about 75 μmol/L; about 25 μmol/L to about 100 μmol/L; about 25 μmol/L to about 125 μmol/L; about 25 μmol/L to about 150 μmol/L; about 25 μmol/L to about 175 μmol/L; about 25 μmol/L to about 75 μmol/L). In some embodiments, the reduction in mean serum bile acid concentration is about 50 μmol/L to about 180 μmol/L relative to baseline (e.g., about 50 μmol/L to about 100 μmol/L; about 50 μmol/L to about 120 μmol/L; about 50 μmol/L to about 150 μmol/L; about 65 μmol/L to about 120 μmol/L; about 50 μmol/L to about 90 μmol/L; about 65 μmol/L to about 85 μmol/L; about 100 μmol/L to about 130 μmol/L; about 100 μmol/L to about 180 μmol/L; or about 150 μmol/L to about 180 μmol/L). In some embodiments, the reduction in mean serum bile acid concentration is about 50 μmol/L to about 600 μmol/L, about 100 μmol/L to about 600 μmol/L, about 150 μmol/L to about 600 μmol/L, about 200 μmol/L to about 600 μmol/L, about 250 μmol/L to about 600 μmol/L, about 300 μmol/L to about 600 μmol/L, about 350 μmol/L to about 600 μmol/L, about 400 μmol/L to about 600 μmol/L, about 450 μmol/L to about 600 μmol/L, about 500 μmol/L to about 600 μmol/L, about 550 μmol/L to about 600 μmol/L, about 50 μmol/L to about 500 μmol/L, about 100 μmol/L to about 500 μmol/L, about 150 μmol/L to about 500 μmol/L, about 200 μmol/L to about 500 μmol/L, about 250 μmol/L to about 500 μmol/L, about 300 μmol/L to about 500 μmol/L, about 350 μmol/L to about 500 μmol/L, about 400 μmol/L to about 500 μmol/L, about 450 μmol/L to about 500 μmol/L, about 50 μmol/L to about 400 μmol/L, about 100 μmol/L to about 400 μmol/L, about 150 μmol/L to about 400 μmol/L, about 200 μmol/L to about 400 μmol/L, about 250 μmol/L to about 400 μmol/L, about 300 μmol/L to about 400 μmol/L, about 350 μmol/L to about 400 μmol/L, about 50 μmol/L to about 300 μmol/L, about 100 μmol/L to about 300 μmol/L, about 150 μmol/L to about 300 μmol/L, about 200 μmol/L to about 300 μmol/L, about 250 μmol/L to about 300 μmol/L, about 50 μmol/L to about 300 μmol/L, about 100 μmol/L to about 300 μmol/L, about 150 μmol/L to about 300 μmol/L, about 200 μmol/L to about 300 μmol/L, about 250 μmol/L to about 300 μmol/L, about 50 μmol/L to about 200 μmol/L, about 100 μmol/L to about 200 μmol/L, or about 150 μmol/L to about 200 μmol/L relative to baseline. In some embodiments, the reduction in mean serum bile acid concentration of about 70 μmol/L to about 120 μmol/L relative to baseline. In some embodiments, the reduction in mean serum bile acid concentration is about 150 μmol/L to about 180 μmol/L.

In some embodiments, the reduction in mean serum bile acid concentration occurs after about 1 week to about 72 weeks of administration of odevixibat, or a pharmaceutically acceptable salt thereof. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of odevixibat, or a pharmaceutically acceptable salt thereof. In some embodiments, the reduction in mean serum bile acid concentration occurs after about 5 weeks to about 8 weeks, about 9 to about 12 weeks, about 21 to about 24 weeks of administration of odevixibat, or a pharmaceutically acceptable salt thereof.

In some embodiments, the reduction in mean serum bile acid concentration occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of odevixibat, or a pharmaceutically acceptable salt thereof.

In some embodiments, the reduction in mean serum bile acid concentration occurs following administration of odevixibat, or a pharmaceutically acceptable salt thereof, for at least 1 week, at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 60 weeks, least 72 weeks, at least 96 weeks, etc. In some embodiments, the reduction in mean serum bile acid concentration occurs following 4 weeks of administration. In some embodiments, the reduction in mean serum bile acid concentration occurs following 12 weeks of administration. In some embodiments, the reduction in mean serum bile acid concentration occurs following 24 weeks of administration. In some embodiments, the reduction in mean serum bile acid concentration occurs following 48 weeks of administration. In some embodiments, the reduction in mean serum bile acid concentration occurs following 72 weeks of administration.

In some embodiments, the reduction in mean serum bile acid concentration is about 50 μmol/L to about 90 μmol/L following 12 weeks of administration of odevixibat, or a pharmaceutically acceptable salt thereof. For example, the reduction in mean serum bile acid concentration is about 65 μmol/L to about 85 μmol/L following 12 weeks of administration of odevixibat, or a pharmaceutically acceptable salt thereof. In some embodiments, the reduction in mean serum bile acid concentration is about 70 μmol/L (e.g., about 73 μmol/L) following 12 weeks of administration of odevixibat, or a pharmaceutically acceptable salt thereof.

In some embodiments, the reduction in mean serum bile acid concentration is about 100 μmol/L to about 130 μmol/L following 24 weeks of administration of odevixibat, or a pharmaceutically acceptable salt thereof. For example, the reduction in mean serum bile acid concentration is about 110 μmol/L to about 120 μmol/L following 24 weeks of administration of odevixibat, or a pharmaceutically acceptable salt thereof. In some embodiments, the reduction in mean serum bile acid concentration is about 115 following 24 weeks of administration of odevixibat, or a pharmaceutically acceptable salt thereof.

In some embodiments, the reduction in mean serum bile acid concentration is about 150 μmol/L to about 180 μmol/L following 48 weeks of administration of odevixibat, or a pharmaceutically acceptable salt thereof. For example, the reduction in mean serum bile acid concentration is about 155 μmol/L to about 170 μmol/L following 48 weeks of administration of odevixibat, or a pharmaceutically acceptable salt thereof. In some embodiments, the reduction in mean serum bile acid concentration is about 165 μmol/L (e.g., about 166 μmol/L) following 48 weeks of administration of odevixibat, or a pharmaceutically acceptable salt thereof.

In some embodiments, the subject exhibits a reduction in bile acid concentration relative to baseline. In some embodiments, the reduction in bile acid concentration is at least 25 μmol/L, at least 50 μmol/L, at least 75 μmol/L, at least 100 μmol/L, at least 125 μmol/L, at least 150 μmol/L, at least 175 μmol/L, at least 200 μmol/L, at least 300 μmol/L, at least 400 μmol/L, at least 500 μmol/L, or at least 600 μmol/L relative to baseline. For example, the reduction in mean bile acid concentration is about 25 μmol/L to about 200 μmol/L relative to baseline (e.g., about 25 μmol/L to about 50 μmol/L; about 25 μmol/L to about 50 μmol/L; about 25 μmol/L to about 75 μmol/L; about 25 μmol/L to about 100 μmol/L; about 25 μmol/L to about 125 μmol/L; about 25 μmol/L to about 150 μmol/L; about 25 μmol/L to about 175 μmol/L; about 25 μmol/L to about 75 μmol/L). In some embodiments, the reduction in bile acid concentration is about 50 μmol/L to about 180 μmol/L relative to baseline (e.g., about 50 μmol/L to about 100 μmol/L; about 50 μmol/L to about 120 μmol/L; about 50 μmol/L to about 150 μmol/L; about 65 μmol/L to about 120 μmol/L; about 50 μmol/L to about 90 μmol/L; about 65 μmol/L to about 85 μmol/L; about 100 μmol/L to about 130 μmol/L; about 100 μmol/L to about 180 μmol/L; or about 150 μmol/L to about 180 μmol/L). In some embodiments, the reduction in bile acid concentration is about 50 μmol/L to about 600 μmol/L, about 100 μmol/L to about 600 μmol/L, about 150 μmol/L to about 600 μmol/L, about 200 μmol/L to about 600 μmol/L, about 250 μmol/L to about 600 μmol/L, about 300 μmol/L to about 600 μmol/L, about 350 μmol/L to about 600 μmol/L, about 400 μmol/L to about 600 μmol/L, about 450 μmol/L to about 600 μmol/L, about 500 μmol/L to about 600 μmol/L, about 550 μmol/L to about 600 μmol/L, about 50 μmol/L to about 500 μmol/L, about 100 μmol/L to about 500 μmol/L, about 150 μmol/L to about 500 μmol/L, about 200 μmol/L to about 500 μmol/L, about 250 μmol/L to about 500 μmol/L, about 300 μmol/L to about 500 μmol/L, about 350 μmol/L to about 500 μmol/L, about 400 μmol/L to about 500 μmol/L, about 450 μmol/L to about 500 μmol/L, about 50 μmol/L to about 400 μmol/L, about 100 μmol/L to about 400 μmol/L, about 150 μmol/L to about 400 μmol/L, about 200 μmol/L to about 400 μmol/L, about 250 μmol/L to about 400 μmol/L, about 300 μmol/L to about 400 μmol/L, about 350 μmol/L to about 400 μmol/L, about 50 μmol/L to about 300 μmol/L, about 100 μmol/L to about 300 μmol/L, about 150 μmol/L to about 300 μmol/L, about 200 μmol/L to about 300 μmol/L, about 250 μmol/L to about 300 μmol/L, about 50 μmol/L to about 300 μmol/L, about 100 μmol/L to about 300 μmol/L, about 150 μmol/L to about 300 μmol/L, about 200 μmol/L to about 300 μmol/L, about 250 μmol/L to about 300 μmol/L, about 50 μmol/L to about 200 μmol/L, about 100 μmol/L to about 200 μmol/L, or about 150 μmol/L to about 200 μmol/L relative to baseline. In some embodiments, the reduction in bile acid concentration of about 70 μmol/L to about 120 μmol/L relative to baseline. In some embodiments, the reduction in bile acid concentration is about 150 μmol/L to about 180 μmol/L.

In some embodiments, the reduction in bile acid concentration occurs after about 1 weeks to about 72 weeks of administration of odevixibat, or a pharmaceutically acceptable salt thereof. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of odevixibat, or a pharmaceutically acceptable salt thereof. In some embodiments, the reduction in bile acid concentration occurs after about 5 weeks to about 8 weeks, about 9 to about 12 weeks, about 21 to about 24 weeks of administration of odevixibat, or a pharmaceutically acceptable salt thereof.

In some embodiments, the subject exhibits a reduction in serum bile acid concentration relative to baseline. For example, the reduction in serum bile acid concentration is at least 25 μmol/L, at least 50 μmol/L, at least 75 μmol/L, at least 100 μmol/L, at least 125 μmol/L, at least 150 μmol/L, at least 175 μmol/L, at least 200 μmol/L, at least 300 μmol/L, at least 400 μmol/L, at least 500 μmol/L, or at least 600 μmol/L relative to baseline. For example, the reduction in mean serum bile acid concentration is about 25 μmol/L to about 200 μmol/L relative to baseline (e.g., about 25 μmol/L to about 50 μmol/L; about 25 μmol/L to about 50 μmol/L; about 25 μmol/L to about 75 μmol/L; about 25 μmol/L to about 100 μmol/L; about 25 μmol/L to about 125 μmol/L; about 25 μmol/L to about 150 μmol/L; about 25 μmol/L to about 175 μmol/L; about 25 μmol/L to about 75 μmol/L). In some embodiments, the reduction in serum bile acid concentration is about 50 μmol/L to about 180 μmol/L relative to baseline (e.g., about 50 μmol/L to about 100 μmol/L; about 50 μmol/L to about 120 μmol/L; about 50 μmol/L to about 150 μmol/L; about 65 μmol/L to about 120 μmol/L; about 50 μmol/L to about 90 μmol/L; about 65 μmol/L to about 85 μmol/L; about 100 μmol/L to about 130 μmol/L; about 100 μmol/L to about 180 μmol/L; or about 150 μmol/L to about 180 μmol/L). In some embodiments, the reduction in serum bile acid concentration is about 50 μmol/L to about 600 μmol/L, about 100 μmol/L to about 600 μmol/L, about 150 μmol/L to about 600 μmol/L, about 200 μmol/L to about 600 μmol/L, about 250 μmol/L to about 600 μmol/L, about 300 μmol/L to about 600 μmol/L, about 350 μmol/L to about 600 μmol/L, about 400 μmol/L to about 600 μmol/L, about 450 μmol/L to about 600 μmol/L, about 500 μmol/L to about 600 μmol/L, about 550 μmol/L to about 600 μmol/L, about 50 μmol/L to about 500 μmol/L, about 100 μmol/L to about 500 μmol/L, about 150 μmol/L to about 500 μmol/L, about 200 μmol/L to about 500 μmol/L, about 250 μmol/L to about 500 μmol/L, about 300 μmol/L to about 500 μmol/L, about 350 μmol/L to about 500 μmol/L, about 400 μmol/L to about 500 μmol/L, about 450 μmol/L to about 500 μmol/L, about 50 μmol/L to about 400 μmol/L, about 100 μmol/L to about 400 μmol/L, about 150 μmol/L to about 400 μmol/L, about 200 μmol/L to about 400 μmol/L, about 250 μmol/L to about 400 μmol/L, about 300 μmol/L to about 400 μmol/L, about 350 μmol/L to about 400 μmol/L, about 50 μmol/L to about 300 μmol/L, about 100 μmol/L to about 300 μmol/L, about 150 μmol/L to about 300 μmol/L, about 200 μmol/L to about 300 μmol/L, about 250 μmol/L to about 300 μmol/L, about 50 μmol/L to about 300 μmol/L, about 100 μmol/L to about 300 μmol/L, about 150 μmol/L to about 300 μmol/L, about 200 μmol/L to about 300 μmol/L, about 250 μmol/L to about 300 μmol/L, about 50 μmol/L to about 200 μmol/L, about 100 μmol/L to about 200 μmol/L, or about 150 μmol/L to about 200 μmol/L relative to baseline. In some embodiments, the reduction in serum bile acid concentration of about 70 μmol/L to about 120 μmol/L relative to baseline. In some embodiments, the reduction in serum bile acid concentration is about 150 μmol/L to about 180 μmol/L.

In some embodiments, the reduction in serum bile acid concentration occurs after about 1 weeks to about 72 weeks of administration of odevixibat, or a pharmaceutically acceptable salt thereof. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of odevixibat, or a pharmaceutically acceptable salt thereof. In some embodiments, the reduction in serum bile acid concentration occurs after about 5 weeks to about 8 weeks, about 9 to about 12 weeks, about 21 to about 24 weeks of administration of odevixibat, or a pharmaceutically acceptable salt thereof.

In some embodiments, the reduction in serum bile acid concentration occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of odevixibat, or a pharmaceutically acceptable salt thereof.

In some embodiments, the reduction in serum bile acid concentration occurs following administration of odevixibat, or a pharmaceutically acceptable salt thereof, for at least 1 week, at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 54 weeks, at least 60 weeks, at least 66 weeks, at least 72 weeks, etc. In some embodiments, the reduction in serum bile acid concentration occurs following 4 weeks of administration. In some embodiments, the reduction in serum bile acid concentration occurs following 12 weeks of administration. In some embodiments, the reduction in serum bile acid concentration occurs following 24 weeks of administration. In some embodiments, the reduction in serum bile acid concentration occurs following 48 weeks of administration. In some embodiments, the reduction in serum bile acid concentration occurs following 72 weeks of administration.

In some embodiments, the reduction in serum bile acid concentration is about 50 μmol/L to about 90 μmol/L following 12 weeks of administration of odevixibat, or a pharmaceutically acceptable salt thereof. For example, the reduction in serum bile acid concentration is about 65 μmol/L to about 85 μmol/L following 12 weeks of administration of odevixibat, or a pharmaceutically acceptable salt thereof. In some embodiments, the reduction in serum bile acid concentration is about 70 μmol/L (e.g., about 73 μmol/L) following 12 weeks of administration of odevixibat, or a pharmaceutically acceptable salt thereof.

In some embodiments, the reduction in serum bile acid concentration is about 100 μmol/L to about 130 μmol/L following 24 weeks of administration of odevixibat, or a pharmaceutically acceptable salt thereof. For example, the reduction in serum bile acid concentration is about 110 μmol/L to about 120 μmol/L following 24 weeks of administration of odevixibat, or a pharmaceutically acceptable salt thereof. In some embodiments, the reduction in serum bile acid concentration is about 115 μmol/L following 24 weeks of administration of odevixibat, or a pharmaceutically acceptable salt thereof.

In some embodiments, the reduction in serum bile acid concentration is about 150 μmol/L to about 180 μmol/L following 48 weeks of administration of odevixibat, or a pharmaceutically acceptable salt thereof. For example, the reduction in serum bile acid concentration is about 155 μmol/L to about 170 μmol/L following 48 weeks of administration of odevixibat, or a pharmaceutically acceptable salt thereof. In some embodiments, the reduction in serum bile acid concentration is about 165 μmol/L (e.g., about 166 μmol/L) following 48 weeks of administration of odevixibat, or a pharmaceutically acceptable salt thereof.

In some embodiments, following administration of odevixibat, or a pharmaceutically acceptable salt thereof, for at least 24 weeks, the subject exhibits a serum bile acid concentration of less than 70 μmol/L (e.g., less than 60 μmol/L; less than 50 μmol/L, etc.).

In some embodiments, following administration of odevixibat, or a pharmaceutically acceptable salt thereof, for at least 24 weeks, the subject exhibits a reduction in serum bile acid concentration of at least 50% relative to baseline (e.g., at least 55%; at least 60; at least 65%; at least 70%; at least 75%; at least 80%; at least 85%; at least 90%; at least 95%). In some embodiments, the subject exhibits a reduction in serum bile acid concentration of at least 60%, at least 70%, or at least 80% relative to baseline.

In some embodiments, the serum bile acid concentration is normalized following administration of odevixibat, or a pharmaceutically acceptable salt thereof. In some embodiments, the serum bile acid concentration is normalized following 48 weeks of administration of odevixibat, or a pharmaceutically acceptable salt thereof.

In some embodiments, following administration of odevixibat or a pharmaceutically acceptable salt thereof, growth is improved relative to placebo. In some embodiments, following administration of odevixibat, or a pharmaceutically acceptable salt thereof, the subject exhibits an increase in mean height Z score relative to baseline.

In some embodiments, the increase in mean height Z score is at least 0.1, at least 0.2, at least 0.5, at least 0.75, at least 1, at least 1.25, or at least 1.5 relative to baseline. For example, the mean height Z score increased about 0.5 to about 2.0 (e.g., about 0.5 to about 0.8; about 0.5 to about 1.2; about 0.5 to about 1.5; about 0.7 to about 1.5; about 0.8 to about 1.4; about 0.9 to about 1.3; or about 1.0 to about 1.2). In some embodiments, the mean height Z score increased about 1.1.

In some embodiments, the increase in mean height Z score occurs after about 20 week to about 72 weeks of administration of odevixibat, or a pharmaceutically acceptable salt thereof. For example, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of odevixibat, or a pharmaceutically acceptable salt thereof. In some embodiments, the increase in mean height Z score occurs after about 21 to about 24 weeks of administration of odevixibat, or a pharmaceutically acceptable salt thereof.

In some embodiments, the increase in mean height Z score occurs after about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of odevixibat, or a pharmaceutically acceptable salt thereof.

In some embodiments, the increase in mean height Z score occurs following administration of odevixibat, or a pharmaceutically acceptable salt thereof, for at least 20 weeks, at least 24 weeks, at least 28 weeks, at least 32 weeks, at least 36 weeks, at least 40 weeks, at least 44 weeks, at least 48 weeks, etc. In some embodiments, the increase in mean height Z score occurs following 24 weeks of administration. In some embodiments, the increase in mean height Z score occurs following 48 weeks of administration. In some embodiments, the increase in mean height Z score occurs following 72 weeks of administration.

In some embodiments, the mean height Z score increases about 0.9 to about 1.3 following administration of odevixibat, or a pharmaceutically acceptable salt thereof, for 48 weeks. In some embodiments, the mean height Z score increases about 1.0 to about 1.2 following administration of odevixibat, or a pharmaceutically acceptable salt thereof, for 48 weeks. In some embodiments, the mean height Z score increases about 1.1 following administration of odevixibat, or a pharmaceutically acceptable salt thereof, for 48 weeks.

In some embodiments, following administration of odevixibat, or a pharmaceutically acceptable salt thereof, the subject exhibits an increase in mean weight Z score.

In some embodiments, the increase in mean weight Z score is at least 0.2, at least 0.4, at least 0.6, at least 0.8, at least 1, at least 1.2, or at least 1.4. For example, the mean weight Z score increased about 0.2 to about 1.5 (e.g., about 0.5 to about 0.8; about 0.5 to about 1.2; about 0.5 to about 1.5; about 0.7 to about 1.5; about 0.8 to about 1.4; about 0.9 to about 1.3; or about 1.0 to about 1.2). In some embodiments, the mean weight Z score increased about 1.1.

In some embodiments, the increase in mean weight Z score occurs after about 1 week to about 72 weeks of administration of odevixibat, or a pharmaceutically acceptable salt thereof. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of odevixibat, or a pharmaceutically acceptable salt thereof. In some embodiments, the increase in mean weight Z score occurs after about 5 weeks to about 8 weeks, about 9 to about 12 weeks, about 21 to about 24 weeks of administration of odevixibat, or a pharmaceutically acceptable salt thereof.

In some embodiments, the increase in mean weight Z score occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of odevixibat, or a pharmaceutically acceptable salt thereof.

In some embodiments, the increase in mean weight Z score occurs following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the increase in mean weight Z score occurs following administration of odevixibat, or a pharmaceutically acceptable salt thereof, for at least 4 weeks, at least 8 weeks, at least 12 weeks, at least 16 weeks, at least 20 weeks, or at least 24 weeks. In some embodiments, the increase in mean weight Z score occurs following 12 weeks of administration. In some embodiments, the increase in mean weight Z score occurs following 24 weeks of administration. In some embodiments, the increase in mean weight Z score occurs following 48 weeks of administration. In some embodiments, the increase in mean weight Z score occurs following 72 weeks of administration.

In some embodiments, the mean weight Z score increases about 0.9 to about 1.3 following administration of odevixibat, or a pharmaceutically acceptable salt thereof, for 48 weeks. In some embodiments, the mean weight Z score increases about 1.0 to about 1.2 following administration of odevixibat, or a pharmaceutically acceptable salt thereof, for 48 weeks. In some embodiments, the mean weight Z score increases about 1.1 following administration of odevixibat, or a pharmaceutically acceptable salt thereof, for 48 weeks.

In some embodiments, following administration of odevixibat, or a pharmaceutically acceptable salt thereof, the subject exhibits an increase in height Z score relative to baseline. In some embodiments, the increase in height Z score is at least 0.1, at least 0.2, at least 0.5, at least 0.75, at least 1, at least 1.25, or at least 1.5 relative to baseline. For example, the height Z score increased about 0.5 to about 2.0 (e.g., about 0.5 to about 0.8; about 0.5 to about 1.2; about 0.5 to about 1.5; about 0.7 to about 1.5; about 0.8 to about 1.4; about 0.9 to about 1.3; or about 1.0 to about 1.2). In some embodiments, the height Z score increased about 1.1.

In some embodiments, the increase in height Z score occurs after about 20 week to about 72 weeks of administration of odevixibat, or a pharmaceutically acceptable salt thereof. For example, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of odevixibat, or a pharmaceutically acceptable salt thereof. In some embodiments, the increase in height Z score occurs after about 21 to about 24 weeks of administration of odevixibat, or a pharmaceutically acceptable salt thereof.

In some embodiments, the increase in height Z score occurs after about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of odevixibat, or a pharmaceutically acceptable salt thereof.

In some embodiments, the increase in height Z score occurs following administration of odevixibat, or a pharmaceutically acceptable salt thereof, for at least 20 weeks, at least 24 weeks, at least 28 weeks, at least 32 weeks, at least 36 weeks, at least 40 weeks, at least 44 weeks, at least 48 weeks, etc. In some embodiments, the increase in height Z score occurs following 24 weeks of administration. In some embodiments, the increase in height Z score occurs following 48 weeks of administration. In some embodiments, the increase in height Z score occurs following 72 weeks of administration.

In some embodiments, the height Z score increases about 0.9 to about 1.3 following administration of odevixibat, or a pharmaceutically acceptable salt thereof, for 48 weeks. In some embodiments, the height Z score increases about 1.0 to about 1.2 following administration of odevixibat, or a pharmaceutically acceptable salt thereof, for 48 weeks. In some embodiments, the height Z score increases about 1.1 following administration of odevixibat, or a pharmaceutically acceptable salt thereof, for 48 weeks.

In some embodiments, following administration of odevixibat, or a pharmaceutically acceptable salt thereof, the subject exhibits an increase in weight Z score.

In some embodiments, the increase in weight Z score is at least 0.2, at least 0.4, at least 0.6, at least 0.8, at least 1, at least 1.2, or at least 1.4. For example, the weight Z score increased about 0.2 to about 1.5 (e.g., about 0.5 to about 0.8; about 0.5 to about 1.2; about 0.5 to about 1.5; about 0.7 to about 1.5; about 0.8 to about 1.4; about 0.9 to about 1.3; or about 1.0 to about 1.2). In some embodiments, the weight Z score increased about 1.1.

In some embodiments, the increase in weight Z score occurs after about 1 week to about 72 weeks of administration of odevixibat, or a pharmaceutically acceptable salt thereof. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of odevixibat, or a pharmaceutically acceptable salt thereof. In some embodiments, the increase in weight Z score occurs after about 5 weeks to about 8 weeks, about 9 to about 12 weeks, about 21 to about 24 weeks of administration of odevixibat, or a pharmaceutically acceptable salt thereof.

In some embodiments, the increase in weight Z score occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of odevixibat, or a pharmaceutically acceptable salt thereof.

In some embodiments, the increase in weight Z score occurs following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the increase in weight Z score occurs following administration of odevixibat, or a pharmaceutically acceptable salt thereof, for at least 4 weeks, at least 8 weeks, at least 12 weeks, at least 16 weeks, at least 20 weeks, or at least 24 weeks. In some embodiments, the increase in weight Z score occurs following 12 weeks of administration. In some embodiments, the increase in weight Z score occurs following 24 weeks of administration. In some embodiments, the increase in weight Z score occurs following 48 weeks of administration. In some embodiments, the increase in weight Z score occurs following 72 weeks of administration.

In some embodiments, the weight Z score increases about 0.9 to about 1.3 following administration of odevixibat, or a pharmaceutically acceptable salt thereof, for 48 weeks. In some embodiments, the weight Z score increases about 1.0 to about 1.2 following administration of odevixibat, or a pharmaceutically acceptable salt thereof, for 48 weeks. In some embodiments, the weight Z score increases about 1.1 following administration of odevixibat, or a pharmaceutically acceptable salt thereof, for 48 weeks.

In some embodiments, the subject exhibits improvement in sleep parameters following administration of odevixibat, or a pharmaceutically acceptable salt thereof. Improvements in sleep parameters can include, for example, decreases in tiredness, caregiver-reported percentage of days with scratching associated with bleeding, needing help falling asleep, needing soothing, sleeping with caregiver, or taking medication to induce sleep, as well as caregiver-reported percentage of days with daytime tiredness, and caregiver-reported number of awakenings per night. As described in the Examples, at week 24, most sleep parameters for patients were significantly improved since starting odevixibat.

In some embodiments, the mean decrease in caregiver-reported percentage of days with scratching associated with bleeding is about 14% to about 45% (e.g., mean decrease of about 15%, about 20%, about 25%, about 30%, about 35%, or about 45%) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the mean decrease in caregiver-reported percentage of days with scratching associated with bleeding is about 14% to about 45% following administration of odevixibat, or a pharmaceutically acceptable salt thereof, for 48 weeks.

In some embodiments, the mean decrease in caregiver-reported percentage of days needing help falling asleep is about 20% to about 75% (e.g., mean decrease of about 22%, about 25%, about 30%, about 35%, about 45%, about 50%, about 55%, about 60%, about 65%, about 70%, or about 75%) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the mean decrease in caregiver-reported percentage of days needing help falling asleep is about 20% to about 75% following administration of odevixibat, or a pharmaceutically acceptable salt thereof, for 48 weeks.

In some embodiments, the mean decrease in caregiver-reported percentage of days needing soothing is about 20% to about 75% (e.g., mean decrease of about 22%, about 25%, about 30%, about 35%, about 45%, about 50%, about 55%, about 60%, about 65%, about 70%, or about 75%) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the mean decrease in caregiver-reported percentage of days needing soothing is about 20% to about 75% following administration of odevixibat, or a pharmaceutically acceptable salt thereof, for 48 weeks.

In some embodiments, the mean decrease in caregiver-reported percentage of days needing to sleep with caregiver is about 20% to about 75% (e.g., mean decrease of about 22%, about 25%, about 30%, about 35%, about 45%, about 50%, about 55%, about 60%, about 65%, about 70%, or about 75%) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the mean decrease in caregiver-reported percentage of days needing to sleep with the caregiver is about 20% to about 75% following administration of odevixibat, or a pharmaceutically acceptable salt thereof, for 48 weeks.

In some embodiments, the mean decrease in caregiver-reported percentage of days with taking medication to induce sleep is about 0.5% to about 10% (e.g., mean decrease of about 1%, about 2%, about 2.5%, about 3%, about 5%, or about 7.5%) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the mean decrease in caregiver-reported percentage of days with taking medication to induce sleep is about 0.5% to about 10% following administration of odevixibat, or a pharmaceutically acceptable salt thereof, for 48 weeks. In some embodiments, the mean decrease in caregiver-reported percentage of days with taking medication to induce sleep is about 1% to about 5% following administration of odevixibat, or a pharmaceutically acceptable salt thereof, for 48 weeks.

In some embodiments, the mean decrease in caregiver-reported amount of days with daytime tiredness is about 0.1 to about 5 (e.g., mean decrease of about 0.1, about 0.5, about 1, about 1.5, about 2, or about 3.5) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the mean decrease in caregiver-reported percentage of days with taking medication to induce sleep is about 0.1 to about 5 following administration of odevixibat, or a pharmaceutically acceptable salt thereof, for 48 weeks. In some embodiments, the mean decrease in caregiver-reported percentage of days with taking medication to induce sleep is about 0.5 to about 1.5 following administration of odevixibat, or a pharmaceutically acceptable salt thereof, for 48 weeks.

In some embodiments, the mean decrease in caregiver-reported number of awakenings per night is about 0.1 to about 7.5 (e.g., mean decrease of about 0.5, about 1, about 2, about 2.5, about 3, or about 5) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the mean decrease in caregiver-reported percentage of days with taking medication to induce sleep is about 0.1 to about 7.5 following administration of odevixibat, or a pharmaceutically acceptable salt thereof, for 48 weeks. In some embodiments, the mean decrease in caregiver-reported percentage of days with taking medication to induce sleep is about 1 to about 5 following administration of odevixibat, or a pharmaceutically acceptable salt thereof, for 48 weeks.

In some embodiments, the decrease in tiredness relative to baseline is at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, at least 3.4, at least 3.5, at least 3.6, at least 3.7, at least 3.8, or at least 3.9. In some embodiments, the decrease in tiredness relative to baseline is about 0.3 to about 4.0. For example, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.5 to about 3.5, about 0.5 to about 3.0, about 0.5 to about 2.5, about 0.5 to about 2.0, about 0.5 to about 1.5, about 0.5 to about 1.0, about 1.0 to about 4.0 about 1.0 to about 3.5, about 1.0 to about 3.0, about 1.0 to about 2.5, about 1.0 to about 2.0, about 1.0 to about 1.5, about 1.5 to about 4.0, about 1.5 to about 3.5, about 1.5 to about 3.0, about 1.5 to about 2.5, about 1.5 to about 2.0, about 2.0 to about 1.0, about 2.0 to about 3.5, about 2.0 to about 3.0, about 2.0 to about 2.5, about 2.5 to about 4.0 about 2.5 to about 3.5, about 2.5 to about 3.0, about 3.0 to about 4.0, about 3.0 to about 3.5, or about 3.5 to about 4.0. In some embodiments, the decrease in tiredness is measured according to a PRO instrument and/or ObsRO instrument.

In some embodiments, the decrease in caregiver-reported percentage of days with scratching associated with bleeding is about 14% to about 45% (e.g., decrease of about 15%, about 20%, about 25%, about 30%, about 35%, or about 45%) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the decrease in caregiver-reported percentage of days with scratching associated with bleeding is about 14% to about 45% following administration of odevixibat, or a pharmaceutically acceptable salt thereof, for 48 weeks.

In some embodiments, the decrease in caregiver-reported percentage of days needing help falling asleep is about 20% to about 75% (e.g., decrease of about 22%, about 25%, about 30%, about 35%, about 45%, about 50%, about 55%, about 60%, about 65%, about 70%, or about 75%) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the decrease in caregiver-reported percentage of days needing help falling asleep is about 20% to about 75% following administration of odevixibat, or a pharmaceutically acceptable salt thereof, for 48 weeks.

In some embodiments, the decrease in caregiver-reported percentage of days needing soothing is about 20% to about 75% (e.g., decrease of about 22%, about 25%, about 30%, about 35%, about 45%, about 50%, about 55%, about 60%, about 65%, about 70%, or about 75%) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the decrease in caregiver-reported percentage of days needing soothing is about 20% to about 75% following administration of odevixibat, or a pharmaceutically acceptable salt thereof, for 48 weeks.

In some embodiments, the decrease in caregiver-reported percentage of days needing to sleep with caregiver is about 20% to about 75% (e.g., decrease of about 22%, about 25%, about 30%, about 35%, about 45%, about 50%, about 55%, about 60%, about 65%, about 70%, or about 75%) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the decrease in caregiver-reported percentage of days needing to sleep with the caregiver is about 20% to about 75% following administration of odevixibat, or a pharmaceutically acceptable salt thereof, for 48 weeks.

In some embodiments, the decrease in caregiver-reported percentage of days with taking medication to induce sleep is about 0.5% to about 10% (e.g., decrease of about 1%, about 2%, about 2.5%, about 3%, about 5%, or about 7.5%) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the decrease in caregiver-reported percentage of days with taking medication to induce sleep is about 0.5% to about 10% following administration of odevixibat, or a pharmaceutically acceptable salt thereof, for 48 weeks. In some embodiments, the decrease in caregiver-reported percentage of days with taking medication to induce sleep is about 1% to about 5% following administration of odevixibat, or a pharmaceutically acceptable salt thereof, for 48 weeks.

In some embodiments, the decrease in caregiver-reported score for daytime tiredness is about 0.1 to about 5 (e.g., decrease of about 0.1, about 0.5, about 1, about 1.5, about 2, or about 3.5) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc.

In some embodiments, the decrease in caregiver-reported percentage of days with taking medication to induce sleep is about 0.1 to about 5 following administration of odevixibat, or a pharmaceutically acceptable salt thereof, for 48 weeks. In some embodiments, the decrease in caregiver-reported percentage of days with taking medication to induce sleep is about 0.5 to about 1.5 following administration of odevixibat, or a pharmaceutically acceptable salt thereof, for 48 weeks.

In some embodiments, the decrease in caregiver-reported number of awakenings per night is about 0.1 to about 7.5 (e.g., decrease of about 0.5, about 1, about 2, about 2.5, about 3, or about 5) following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. In some embodiments, the decrease in caregiver-reported percentage of days with taking medication to induce sleep is about 0.1 to about 7.5 following administration of odevixibat, or a pharmaceutically acceptable salt thereof, for 48 weeks. In some embodiments, the decrease in caregiver-reported percentage of days with taking medication to induce sleep is about 1 to about 5 following administration of odevixibat, or a pharmaceutically acceptable salt thereof, for 48 weeks.

In some embodiments, the subject exhibits improvement in liver parameters or liver biomarkers following administration of odevixibat, or a pharmaceutically acceptable salt thereof. Non-limiting examples of biomarkers indicative of one or more of liver damage, liver inflammation, liver fibrosis, and/or liver cirrhosis include alanine transaminase (ALT) levels, aspartate transaminase (AST) levels, alkaline phosphatase (ALP) levels, gamma-glutamyl transferase (GGT) levels, total and direct bilirubin levels, autotaxin levels, prothrombin time (PT), the international normalized ratio (INR), and total protein and albumin (see, e.g., Lala et al., “Liver Function Tests.” StatPearls, StatPearls Publishing, 5 Oct. 2022 (PMID: 29494096).

For example, in some embodiments, levels of autotaxin, which is linked to cholestatic pruritus intensity, and/or plasma 7α-hydroxy-4-cholesten-3-one (p-C4), a marker of bile acid synthesis, are improved following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc.

In some embodiments, autotaxin levels are decreased following administration of odevixibat, or a pharmaceutically acceptable salt thereof. In some embodiments, autotaxin levels can be decreased about 10 to about 1000 ng/mL from baseline following administration of odevixibat, or a pharmaceutically acceptable salt thereof. For example, autotaxin levels can be decreased about 10 to about 100 ng/mL, about 10 to about 200 ng/mL, about 10 to about 300 ng/mL, about 10 to about 400 ng/mL, about 10 to about 500 ng/mL, about 10 to about 600 ng/mL, about 10 to about 700 ng/mL, about 10 to about 800 ng/mL, about 10 to about 900 ng/mL, about 100 to about 200 ng/mL, about 100 to about 300 ng/mL, about 100 to about 400 ng/mL, about 100 to about 500 ng/mL, about 100 to about 600 ng/mL, about 100 to about 700 ng/mL, about 100 to about 800 ng/mL, about 100 to about 900 ng/mL, about 100 to about 1000 ng/mL, about 200 to about 300 ng/mL, about 200 to about 400 ng/mL, about 200 to about 500 ng/mL, about 200 to about 600 ng/mL, about 200 to about 700 ng/mL, about 200 to about 800 ng/mL, about 200 to about 900 ng/mL, about 200 to about 1000 ng/mL, about 300 to about 400 ng/mL, about 300 to about 500 ng/mL, about 300 to about 600 ng/mL, about 300 to about 700 ng/mL, about 300 to about 800 ng/mL, about 300 to about 900 ng/mL, about 300 to about 1000 ng/mL, about 400 to about 500 ng/mL, about 400 to about 600 ng/mL, about 400 to about 700 ng/mL, about 400 to about 800 ng/mL, about 400 to about 900 ng/mL, about 400 to about 1000 ng/mL, about 500 to about 600 ng/mL, about 500 to about 700 ng/mL, about 500 to about 800 ng/mL, about 500 to about 900 ng/mL, about 500 to about 1000 ng/mL, about 600 to about 700 ng/mL, about 600 to about 800 ng/mL, about 600 to about 900 ng/mL, about 600 to about 1000 ng/mL, about 700 to about 800 ng/mL, about 700 to about 900 ng/mL, about 700 to about 1000 ng/mL, about 800 to about 900 ng/mL, about 800 to about 1000 ng/mL, or about 900 to about 1000 ng/mL from baseline following administration of odevixibat, or a pharmaceutically acceptable salt thereof.

In some embodiments, autotaxin levels can be decreased about 500 to about 1000 ng/mL, about 750 to about 1500 ng/mL, about 1000 to about 2000 ng/mL, or about 1500 to about 2500 ng/mL from baseline following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, autoxtaxin levels can be reduced approximately 50% following administration of odevixibat, or a pharmaceutically acceptable salt thereof, for at least 24 weeks.

In some embodiments, plasma C4 levels are increased following administration of odevixibat, or a pharmaceutically acceptable salt thereof. In some embodiments, plasma C4 levels (ng/mL) can be increased about 1 to about 30 ng/mL from baseline following administration of odevixibat, or a pharmaceutically acceptable salt thereof. For example, about 1 to about 5 ng/mL, about 1 to about 10 ng/mL, about 1 to about 15 ng/mL, about 1 to about 20 ng/mL, about 1 to about 25 ng/mL, about 5 to about 10 ng/mL, about 5 to about 15 ng/mL, about 5 to about 20 ng/mL, about 5 to about 25 ng/mL, about 5 to about 30 ng/mL, about 10 to about 15 ng/mL, about 10 to about 20 ng/mL, about 10 to about 25 ng/mL, about 10 to about 30 ng/mL, about 15 to about 20 ng/mL, about 15 to about 25 ng/mL, about 15 to about 30 ng/mL, about 20 to about 25 ng/mL, about 20 to about 30 ng/mL, or about 25 to about 30 ng/mL from baseline following administration of odevixibat, or a pharmaceutically acceptable salt thereof.

In some embodiments, plasma C4 levels (ng/mL) can be increased 7.5 to 15 ng/mL, 10 to 20 ng/mL, 15 to 25 ng/mL, 20 to 30 ng/mL, or 25 to 35 ng/mL from baseline following administration of odevixibat, or a pharmaceutically acceptable salt thereof, for at least 4 weeks, at least 12 weeks, at least 24 weeks, at least 36 weeks, at least 48 weeks, at least 72 weeks, at least 96 weeks, etc.

In some embodiments, serum alanine aminotransferase (ALT) levels are improved following administration of odevixibat, or a pharmaceutically acceptable salt thereof. In some embodiments, ALT levels are decreased following administration of odevixibat, or a pharmaceutically acceptable salt thereof. In some embodiments, ALT levels are decreased about 10 U/L to about 125 U/L from baseline following administration of odevixibat, or a pharmaceutically acceptable salt thereof. For example, about 10 U/L to about 25 U/L, about 10 U/L to about 50 U/L, about 10 U/L to about 75 U/L, about 25 U/L to about 50 U/L, about 25 U/L to about 75 U/L, about 25 U/L to about 100 U/L, about 50 U/L to about 75 U/L, about 50 U/L to about 100 U/L, or about 75 U/L to about 100 U/L from baseline following administration of odevixibat, or a pharmaceutically acceptable salt thereof.

In some embodiments, ALT levels are decreased about 50 U/L to about 175 U/L, about 50 U/L to about 150 U/L, about 50 U/L to about 125 U/L, or about 100 U/L to about 150 U/L from baseline following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. For example, ALT levels can be reduced approximately 70% following administration of odevixibat, or a pharmaceutically acceptable salt thereof, for at least 21-24 weeks.

In some embodiments, total bilirubin levels are decreased following administration of odevixibat, or a pharmaceutically acceptable salt thereof. In some embodiments, total bilirubin levels are decreased about 0.5 mg/dL to about 5.5 mg/dL, about 1 mg/dL to about 5.5 mg/dL, about 1.5 mg/dL to about 5.5 mg/dL, about 2 mg/dL to about 5.5 mg/dL, about 2.5 mg/dL to about 5.5 mg/dL, about 3 mg/dL to about 5.5 mg/dL, about 3.5 mg/dL to about 5.5 mg/dL, about 4 mg/dL to about 5.5 mg/dL, about 4.5 mg/dL to about 5.5 mg/dL, about 5 mg/dL to about 5.5 mg/dL, about 1 mg/dL to about 5 mg/dL, about 1.5 mg/dL to about 5 mg/dL, about 2 mg/dL to about 5 mg/dL, about 2.5 mg/dL to about 5 mg/dL, about 3 mg/dL to about 5 mg/dL, about 3.5 mg/dL to about 5 mg/dL, about 4 mg/dL to about 5 mg/dL, about 4.5 mg/dL to about 5 mg/dL, about 1 mg/dL to about 4.5 mg/dL, about 1.5 mg/dL to about 4.5 mg/dL, about 2 mg/dL to about 4.5 mg/dL, about 2.5 mg/dL to about 4.5 mg/dL, about 3 mg/dL to about 4.5 mg/dL, about 3.5 mg/dL to about 4.5 mg/dL, about 4 mg/dL to about 4.5 mg/dL, about 1 mg/dL to about 4 mg/dL, about 1.5 mg/dL to about 4 mg/dL, about 2 mg/dL to about 4 mg/dL, about 2.5 mg/dL to about 4 mg/dL, about 3 mg/dL to about 4 mg/dL, about 3.5 mg/dL to about 4 mg/dL, about 1 mg/dL to about 3.5 mg/dL, about 1.5 mg/dL to about 3.5 mg/dL, about 2 mg/dL to about 3.5 mg/dL, about 2.5 mg/dL to about 3.5 mg/dL, about 3 mg/dL to about 3.5 mg/dL, about 1 mg/dL to about 3 mg/dL, about 1.5 mg/dL to about 3 mg/dL, about 2 mg/dL to about 3 mg/dL, about 2.5 mg/dL to about 3 mg/dL, about 1 mg/dL to about 2.5 mg/dL, about 1.5 mg/dL to about 2.5 mg/dL, about 2 mg/dL to about 2.5 mg/dL, about 1 mg/dL to about 2 mg/dL, about 1.5 mg/dL to about 2 mg/dL, or about 1 mg/dL to about 1.5 mg/dL from baseline following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, total bilirubin can be reduced at least 70% (e.g., approximately 99%) following administration of odevixibat, or a pharmaceutically acceptable salt thereof, for at least 24 weeks.

In some embodiments, total bilirubin levels are decreased about 0.5 mg/dL to about 3.5 mg/dL, about 1 mg/dL to about 3.5 mg/dL, about 1 mg/dL to about 3 mg/dL, or about 1.5 mg/dL to about 3.1 mg/dL from baseline following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. For example, total bilirubin can be reduced at least 70% (e.g., approximately 99%) following administration of odevixibat, or a pharmaceutically acceptable salt thereof, for at least 24 weeks.

In some embodiments, serum aspartate aminotransferase (AST) levels are improved following administration of odevixibat, or a pharmaceutically acceptable salt thereof.

In some embodiments, following administration of odevixibat, or a pharmaceutically acceptable salt thereof, the subject exhibits a reduction in clinician xanthoma score. The clinician xanthoma score ranges from 0 to 4, with higher scores (e.g., 4 or 3) indicating more lesions and greater interference with activities, and lower scores indicating no/less lesions and no/less interference with activities.

In some embodiments, the reduction in clinician xanthoma score is at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.2, at least 2.4, at least 2.6, at least 2.8, at least, 3.0, at least 3.2, at least 3.4, at least 3.6, at least 3.8, or at least 4.0 from baseline following administration of odevixibat, or a pharmaceutically acceptable salt thereof. In some embodiments, the reduction in clinician xanthoma score is about 0.3 to about 4.0 For example, about 0.3 to about 1.0, about 0.3 to about 2.0, about 0.3 to about 3.0, about 1.0 to about 2.0, about 1.0 to about 3.0, about 1.0 to about 4.0, about 2.0 to about 3.0, about 2.0 to about 4.0, or about 3.0 to about 4.0 from baseline following administration of odevixibat, or a pharmaceutically acceptable salt thereof. In some embodiments, the reduction in clinician xanthoma score is about 0.3 to about 2.0 (e.g., about 0.5 to about 1.5; about 0.5 to about 1.2; about 0.5 to about 1; or about 0.5 to about 0.8). In some embodiments, the reduction in clinician xanthoma score is about 0.5. In some embodiments, the reduction in clinician xanthoma score is about 0.6.

In some embodiments, the reduction in clinician xanthoma score occurs following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. For example, the reduction in clinician xanthoma score can occur following administration of odevixibat, or a pharmaceutically acceptable salt thereof, for at least 4 weeks, at least 8 weeks, at least 12 weeks, at least 16 weeks, at least 20 weeks, or at least 24 weeks. In some embodiments, the reduction in clinician xanthoma score occurs following 4 weeks of administration. In some embodiments, the reduction in clinician xanthoma score occurs following 24 weeks of administration. In some embodiments, the reduction in clinician xanthoma score occurs following 48 weeks of administration. In some embodiments, the reduction in clinician xanthoma score occurs following 72 weeks of administration.

In some embodiments, the reduction in clinician xanthoma score is about 0.4 to about 1 following 24 weeks of administration of odevixibat, or a pharmaceutically acceptable salt thereof. For example, the reduction in clinician xanthoma score is about 0.5 to about 0.8 following 24 weeks of administration of odevixibat, or a pharmaceutically acceptable salt thereof. In some embodiments, the reduction in clinician xanthoma score is about 0.6 following 24 weeks of administration of odevixibat, or a pharmaceutically acceptable salt thereof.

In some embodiments, the reduction in clinician xanthoma score is about 0.5 to about 2.0 following 48 weeks of administration of odevixibat, or a pharmaceutically acceptable salt thereof. For example, the reduction in clinician xanthoma score is about 0.8 to about 1.5 following 48 weeks of administration of odevixibat, or a pharmaceutically acceptable salt thereof.

In some embodiments, following administration of odevixibat, or a pharmaceutically acceptable salt thereof, the subject exhibits a reduction in clinician xanthoma score relative to baseline. In some embodiments, the reduction in clinician xanthoma score relative to baseline is at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0. For example, the reduction in clinician xanthoma score relative to baseline is about 0.3 to about 2.0 (e.g., about 0.5 to about 1.5; about 0.5 to about 1.2; about 0.5 to about 1; or about 0.5 to about 0.8). In some embodiments, the reduction in clinician xanthoma score relative to baseline is about 0.5. In some embodiments, the reduction in clinician xanthoma score is about 0.6.

In some embodiments, the reduction in clinician xanthoma score relative to baseline occurs following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 72 weeks, at least 96 weeks, etc. For example, the reduction in clinician xanthoma score relative to baseline can occur following administration of odevixibat, or a pharmaceutically acceptable salt thereof, for at least 4 weeks, at least 8 weeks, at least 12 weeks, at least 16 weeks, at least 20 weeks, or at least 24 weeks. In some embodiments, the reduction in clinician xanthoma score relative to baseline occurs following 4 weeks of administration. In some embodiments, the reduction in clinician xanthoma score relative to baseline occurs following 24 weeks of administration. In some embodiments, the reduction in clinician xanthoma score relative to baseline occurs following 48 weeks of administration. In some embodiments, the reduction in clinician xanthoma score relative to baseline occurs following 72 weeks of administration.

In some embodiments, the reduction in clinician xanthoma score relative to baseline is about 0.4 to about 1 following 24 weeks of administration of odevixibat, or a pharmaceutically acceptable salt thereof. For example, the reduction in clinician xanthoma score relative to baseline is about 0.5 to about 0.8 following 24 weeks of administration of odevixibat, or a pharmaceutically acceptable salt thereof. In some embodiments, the reduction in clinician xanthoma score relative to baseline is about 0.6 following 24 weeks of administration of odevixibat, or a pharmaceutically acceptable salt thereof.

In some embodiments, the reduction in clinician xanthoma score relative to baseline is about 0.5 to about 2.0 following 48 weeks of administration of odevixibat, or a pharmaceutically acceptable salt thereof. For example, the reduction in clinician xanthoma score relative to baseline is about 0.8 to about 1.5 following 48 weeks of administration of odevixibat, or a pharmaceutically acceptable salt thereof.

In some embodiments, following administration of odevixibat, or a pharmaceutically acceptable salt thereof, the subject exhibits a reduction in serum cholesterol levels (mmol/L).

In some embodiments, the reduction in cholesterol level is at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, or at least 2.2 mmol/L. For example, the reduction in cholesterol level is about 0.2 to about 2.5 mmol/L (e.g., about 0.2 to about 2.0; about 0.2 to about 1.5; about 0.2 to about 1.2; about 0.2 to about 1.0; about 0.2 to about 0.8; about 0.2 to about 0.5; about 0.5 to about 2.5; about 0.5 to about 2.0; about 0.5 to about 1.5; about 0.5 to about 1.2; about 0.5 to about 1.0; about 0.5 to about 0.8; about 1.0 to about 2.5; about 1.0 to about 2.0; about 1.0 to about 1.5; about 1.0 to about 1.2; about 1.2 to about 2.5; about 1.2 to about 2.0; about 1.2 to about 1.5; about 1.5 to about 2.5; about 1.5 to about 2.0; or about 2.0 to about 2.5 mmol/L). In some embodiments, the reduction in cholesterol level is about 0.5 mmol/L. In some embodiments, the reduction in cholesterol level is about 0.6 mmol/L.

In some embodiments, the reduction in cholesterol level occurs after about 1 week to about 72 weeks of administration of odevixibat, or a pharmaceutically acceptable salt thereof. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of odevixibat, or a pharmaceutically acceptable salt thereof. In some embodiments, the reduction in cholesterol level occurs after about 5 weeks to about 8 weeks, about 9 to about 12 weeks, about 21 to about 24 weeks of administration of odevixibat, or a pharmaceutically acceptable salt thereof.

In some embodiments, the reduction in cholesterol level occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of odevixibat, or a pharmaceutically acceptable salt thereof.

In some embodiments, the reduction in cholesterol level occurs following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the reduction in cholesterol level can occur following administration of odevixibat, or a pharmaceutically acceptable salt thereof, for at least 4 weeks, at least 8 weeks, at least 12 weeks, at least 16 weeks, at least 20 weeks, or at least 24 weeks. In some embodiments, the reduction in cholesterol level occurs following 4 weeks of administration. In some embodiments, the reduction in cholesterol level occurs following 24 weeks of administration. In some embodiments, the reduction in cholesterol level occurs following 48 weeks of administration. In some embodiments, the reduction in cholesterol level occurs following 72 weeks of administration.

In some embodiments, the reduction in cholesterol level is about 0.4 to about 1 mmol/L following 24 weeks of administration of odevixibat, or a pharmaceutically acceptable salt thereof. For example, the reduction in cholesterol level is about 0.5 to about 0.8 mmol/L following 24 weeks of administration of odevixibat, or a pharmaceutically acceptable salt thereof. In some embodiments, the reduction in cholesterol level is about 0.6 mmol/L following 24 weeks of administration of odevixibat, or a pharmaceutically acceptable salt thereof.

In some embodiments, the reduction in cholesterol level is about 0.5 to about 2.0 mmol/L following 48 weeks of administration of odevixibat, or a pharmaceutically acceptable salt thereof. For example, the reduction in cholesterol level is about 0.8 to about 1.5 mmol/L following 48 weeks of administration of odevixibat, or a pharmaceutically acceptable salt thereof.

In some embodiments, following administration of odevixibat, or a pharmaceutically acceptable salt thereof, the subject exhibits a reduction in cholesterol level relative to baseline. In some embodiments, the reduction in cholesterol level relative to baseline is at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0 mmol/L. For example, the reduction in cholesterol level relative to baseline is about 0.3 to about 2.0 mmol/L (e.g., about 0.5 to about 1.5; about 0.5 to about 1.2; about 0.5 to about 1; or about 0.5 to about 0.8 mmol/L). In some embodiments, the reduction in cholesterol level relative to baseline is about 0.5 mmol/L. In some embodiments, the reduction in cholesterol level is about 0.6 mmol/L.

In some embodiments, the reduction in cholesterol level relative to baseline occurs following administration of odevixibat, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the reduction in cholesterol level relative to baseline can occur following administration of odevixibat, or a pharmaceutically acceptable salt thereof, for at least 4 weeks, at least 8 weeks, at least 12 weeks, at least 16 weeks, at least 20 weeks, or at least 24 weeks. In some embodiments, the reduction in cholesterol level relative to baseline occurs following 4 weeks of administration. In some embodiments, the reduction in cholesterol level relative to baseline occurs following 24 weeks of administration. In some embodiments, the reduction in cholesterol level relative to baseline occurs following 48 weeks of administration. In some embodiments, the reduction in cholesterol level relative to baseline occurs following 72 weeks of administration.

In some embodiments, the reduction in cholesterol level relative to baseline is about 0.4 to about 1 mmol/L following 24 weeks of administration of odevixibat, or a pharmaceutically acceptable salt thereof. For example, the reduction in cholesterol level relative to baseline is about 0.5 to about 0.8 mmol/L following 24 weeks of administration of odevixibat, or a pharmaceutically acceptable salt thereof. In some embodiments, the reduction in cholesterol level relative to baseline is about 0.6 mmol/L following 24 weeks of administration of odevixibat, or a pharmaceutically acceptable salt thereof.

In some embodiments, the reduction in cholesterol level relative to baseline is about 0.5 to about 2.0 mmol/L following 48 weeks of administration of odevixibat, or a pharmaceutically acceptable salt thereof. For example, the reduction in cholesterol level relative to baseline is about 0.8 to about 1.5 mmol/L following 48 weeks of administration of odevixibat, or a pharmaceutically acceptable salt thereof.

In some embodiments, the subject is a pediatric subject. In some embodiments, the subject is not a pediatric subject. In some embodiments, the subject is an adult subject.

In some embodiments, a subject has a decrease in scratching score of 1 or more points after administration of odevixibat, or a pharmaceutically acceptable salt thereof, relative to baseline.

In some embodiments, the subject is a pruritus responder. For example, the subject has a decrease or one or more points in scratching score after administration of odevixibat, or a pharmaceutically acceptable salt thereof, relative to baseline. In some embodiments, a pruritus responder has a decrease in scratching score of 1 or more points after administration of odevixibat, or a pharmaceutically acceptable salt thereof, for 21 to 24 weeks (e.g., 21, 22, 23, and/or 24 weeks) relative to baseline. In some embodiments, a pruritus responder has a scratching score averaged at weeks 20-24 of odevixibat administration that is one or more points decreased relative to baseline. In some embodiments, the scratching score as measured using the PRUCISION™ ObsRO instrument. In some embodiments, the subject is administered about 20 to about 800 μg/kg/day of odevixibat, or a pharmaceutically acceptable salt thereof. For example, about 20 to about 600, about 20 to about 400, about 20 to about 200, about 20 to about 180, about 20 to about 160, about 20 to about 140, about 20 to about 120, about 20 to about 100, about 20 to about 80, about 20 to about 60, about 20 to about 40, about 40 to about 800, about 40 to about 600, about 40 to about 400, about 40 to about 200, about 40 to about 180, about 40 to about 160, about 40 to about 140, about 40 to about 120, about 40 to about 100, about 40 to about 80, about 40 to about 60, about 60 to about 800, about 60 to about 600, about 60 to about 400, about 60 to about 200, about 60 to about 180, about 60 to about 160, about 60 to about 140, about 60 to about 120, about 60 to about 100, about 60 to about 80, about 80 to about 800, about 80 to about 600, about 80 to about 400, about 80 to about 200, about 80 to about 180, about 80 to about 160, about 80 to about 140, about 80 to about 120, about 80 to about 100, about 100 to about 800, about 100 to about 600, about 100 to about 400, about 100 to about 200, about 100 to about 180, about 100 to about 160, about 100 to about 140, about 100 to about 120, about 120 to about 800, about 120 to about 600, about 120 to about 400, about 120 to about 200, about 120 to about 180, about 120 to about 160, about 120 to about 140, about 140 to about 800, about 140 to about 600, about 140 to about 400, about 140 to about 200, about 140 to about 180, about 140 to about 160, about 160 to about 800, about 160 to about 600, about 160 to about 400, about 160 to about 200, about 160 to about 180, about 180 to about 800, about 180 to about 600, about 180 to about 400, about 180 to about 200, about 200 to about 800, about 200 to about 600, about 200 to about 400, about 400 to about 800, about 400 to about 600, or about 600 to about 800 μg/kg/day of odevixibat, or a pharmaceutically acceptable salt thereof. In some embodiments, the subject is administered about 20, about 30, about 40, about 50, about 60, about 70, about 80, about 90, about 100, about 120, about 140, about 150, about 160, about 170, about 180, about 190, about 200, about 400, about 600, or about 800 μg/kg/day of odevixibat, or a pharmaceutically acceptable salt thereof. In some embodiments, the subject is administered 120 μg/kg/day of odevixibat, or a pharmaceutically acceptable salt thereof. In some embodiments, the subject is administered 800 μg/kg/day of odevixibat, or a pharmaceutically acceptable salt thereof.

In some embodiments, the subject is administered odevixibat, or a pharmaceutically acceptable salt thereof, in an amount that does not exceed 6 mg per day.

In some embodiments, odevixibat, or a pharmaceutically acceptable salt thereof, is administered as a unit dose ranging from about 1 μg to about 100 mg, such as from about 10 μg to about 10 mg, such as from about 100 μg to about 2000 μg, or such as from about 200 μg to about 1500 μg. In some embodiments, odevixibat, or a pharmaceutically acceptable salt thereof, is administered as a unit dose ranging from about 10 μg to about 9 mg, about 10 μg to about 8 mg, about 10 μg to about 7 mg, about 10 μg to about 6 mg, about 10 μg to about 5 mg, about 10 μg to about 4 mg, about 10 μg to about 3 mg, about 10 μg to about 2 mg, about 10 μg to about 1 mg, about 10 μg to about 800 μg, about 10 μg to about 600 μg, about 10 μg to about 400 μg, about 10 μg to about 200 μg, about 10 μg to about 100 μg, about 10 μg to about 50 μg, about 50 μg to about 10 mg, about 50 μg to about 9 mg, about 50 μg to about 8 mg, about 50 μg to about 7 mg, about 50 μg to about 6 mg, about 50 μg to about 5 mg, about 50 μg to about 4 mg, about 50 μg to about 3 mg, about 50 μg to about 2 mg, about 50 μg to about 1 mg, about 50 μg to about 800 μg, about 50 μg to about 600 μg, about 50 μg to about 400 μg, about 50 μg to about 200 μg, about 50 μg to about 100 μg, about 100 μg to about 10 mg, about 100 μg to about 9 mg, about 100 μg to about 8 mg, about 100 μg to about 7 mg, about 100 μg to about 6 mg, about 100 μg to about 5 mg, about 100 μg to about 4 mg, about 100 μg to about 3 mg, about 100 μg to about 2 mg, about 100 μg to about 1 mg, about 100 μg to about 800 μg, about 100 μg to about 600 μg, about 100 μg to about 400 μg, about 100 μg to about 200 μg, about 200 μg to about 10 mg, about 200 μg to about 9 mg, about 200 μg to about 8 mg, about 200 μg to about 7 mg, about 200 μg to about 6 mg, about 200 μg to about 5 mg, about 200 μg to about 4 mg, about 200 μg to about 3 mg, about 200 μg to about 2 mg, about 200 μg to about 1 mg, about 200 μg to about 800 μg, about 200 μg to about 600 μg, about 200 μg to about 400 μg, about 200 μg to about 10 mg, about 200 μg to about 9 mg, about 400 μg to about 8 mg, about 400 μg to about 7 mg, about 400 μg to about 6 mg, about 400 μg to about 5 mg, about 400 μg to about 4 mg, about 400 μg to about 3 mg, about 400 μg to about 2 mg, about 400 μg to about 1 mg, about 400 μg to about 800 μg, about 400 μg to about 600 μg, about 600 μg to about 10 mg, about 600 μg to about 9 mg, about 600 μg to about 8 mg, about 600 μg to about 7 mg, about 600 μg to about 6 mg, about 600 μg to about 5 mg, about 600 μg to about 4 mg, about 600 μg to about 3 mg, about 600 μg to about 2 mg, about 600 μg to about 1 mg, about 600 μg to about 800 μg, about 800 μg to about 10 mg, about 800 μg to about 9 mg, about 800 μg to about 8 mg, about 800 μg to about 7 mg, about 800 μg to about 6 mg, about 800 μg to about 5 mg, about 800 μg to about 4 mg, about 800 μg to about 3 mg, about 800 μg to about 2 mg, about 800 μg to about 1 mg, about 1 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4 mg, about 1 mg to about 3 mg, about 1 mg to about 2 mg, about 2 mg to about 10 mg, about 2 mg to about 9 mg, about 2 mg to about 8 mg, about 2 mg to about 7 mg, about 2 mg to about 6 mg, about 2 mg to about 5 mg, about 2 mg to about 4 mg, about 2 mg to about 3 mg, about 3 mg to about 10 mg, about 3 mg to about 9 mg, about 3 mg to about 8 mg, about 3 mg to about 7 mg, about 3 mg to about 6 mg, about 3 mg to about 5 mg, about 3 mg to about 4 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 10 mg, about 5 mg to about 9 mg, about 5 mg to about 8 mg, about 5 mg to about 7 mg, about 5 mg to about 6 mg, about 6 mg to about 10 mg, about 6 mg to about 9 mg, about 6 mg to about 8 mg, about 6 mg to about 7 mg, about 7 mg to about 10 mg, about 7 mg to about 9 mg, about 7 mg to about 8 mg, about 8 mg to about 10 mg, about 8 mg to about 9 mg, or about 9 mg to about 10 mg, In some embodiments, odevixibat, or a pharmaceutically acceptable salt thereof, is administered as a unit dose of about 100 μg, about 200 μg, about 300 μg, about 400 μg, about 500 μg, about 600 μg, about 700 μg, about 800 μg, about 900 μg, about 1000 μg, about 1100 μg, about 1200 μg, about 1300 μg, about 1400 μg, about 1500 μg, about 1600 μg, about 1700 μg, about 1800 μg, about 1900 μg, or about 2000 μg.

In some embodiments, odevixibat, or a pharmaceutically acceptable salt thereof, is administered as a unit dose of about 200 μg. In some embodiments, odevixibat, or a pharmaceutically acceptable salt thereof, is administered as a unit dose of about 400 μg. In some embodiments, odevixibat, or a pharmaceutically acceptable salt thereof, is administered as a unit dose of about 600 μg. In some embodiments, odevixibat, or a pharmaceutically acceptable salt thereof, is administered as a unit dose of about 1200 μg.

The frequency of administration can vary from once or twice a week to once or more times a day, such as two or three times daily. In some embodiments, odevixibat, or a pharmaceutically acceptable salt thereof, is administered once daily. The frequency of administration can furthermore remain constant or be variable during the duration of the treatment. Several factors can influence the frequency of administration and the effective amount of the formulation that should be used for a particular treatment, such as the severity of the condition being treated, the duration of the treatment, as well as the age, weight, sex, diet and general medical condition of the patient being treated.

In some embodiments, odevixibat, or a pharmaceutically acceptable salt thereof, is administered as a unit dose of about 200 μg per day. In some embodiments, odevixibat, or a pharmaceutically acceptable salt thereof, is administered as a unit dose of about 400 μg per day. In some embodiments, odevixibat, or a pharmaceutically acceptable salt thereof, is administered as a unit dose of about 600 μg per day. In some embodiments, odevixibat, or a pharmaceutically acceptable salt thereof, is administered as a unit dose of about 1200 μg per day.

In some embodiments, the subject was odevixibat naïve prior to the first administration of the pharmaceutical formulation comprising odevixibat, or a pharmaceutically acceptable salt thereof.

In some embodiments, the subject was administered one or more antiprurituc agents prior to the first administration of a pharmaceutical formulation comprising odevixibat, or a pharmaceutically acceptable salt thereof. Non-limiting examples of an antiprurituc agent include cholestyramine, ursodeoxycholic acid (UDCA), rifampicin, phenobarbital, ondansetron, sertraline, and naltrexone.

In some embodiments, the subject has a scratching score of ______ prior to the first administration of a pharmaceutical formulation comprising odevixibat, or a pharmaceutically acceptable salt thereof.

### IBAT Inhibitors

Provided herein are methods for treating ALGS with an ileal bile acid transport (IBAT) inhibitor (also referred to as an apical sodium-dependent bile acid transport inhibitor ASBTI). In some embodiments, the IBAT inhibitor is

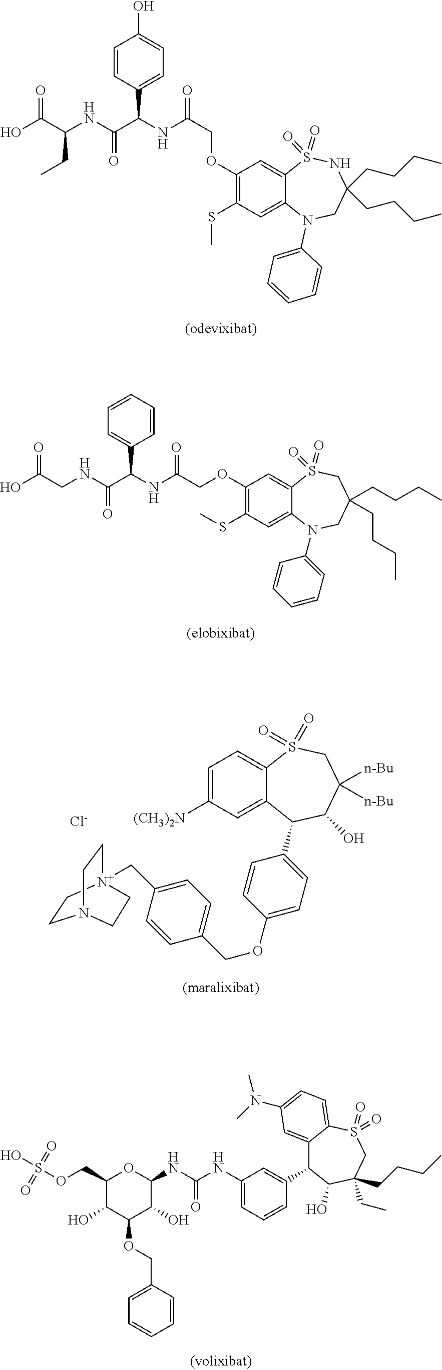

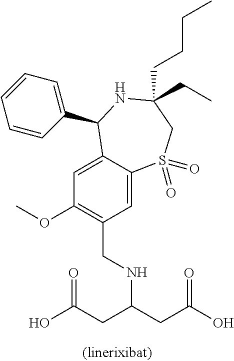 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof. An IBAT inhibitor as provided herein includes solvates and hydrates thereof. For example, odevixibat can be present as a hydrate (e.g., a sesquihydrate). In some embodiments, the IBAT inhibitor is odevixibat, or a pharmaceutically acceptable salt thereof. In some embodiments, the IBAT inhibitor is maralixibat, or a pharmaceutically acceptable salt thereof. In some embodiments, the IBAT inhibitor is volixibat, or a pharmaceutically acceptable salt thereof. In some embodiments, the IBAT inhibitor is elobixibat, or a pharmaceutically acceptable salt thereof. In some embodiments, the IBAT inhibitor is linerixibat, or a pharmaceutically acceptable salt thereof. In some embodiments, the IBAT inhibitor comprises a combination of two or more of odevixibat, maralixibat, volixibat, elobixibat, and linerixibat, or a pharmaceutically acceptable salt thereof.

IBAT inhibitors can be prepared using described methods, for example, U.S. Pat. Nos. 5,994,391; 6,020,330; 6,906,058; 7,192,945; 7,132,416; 7,238,684; and International Publication No. WO 96/05188. The IBAT inhibitor can be present in amorphous or crystalline form. See, for example, U.S. Pat. Nos. 9,409,875; 10,183,920; and International Publication No. WO 2019/245448.

Provided herein are methods for treating Alagille syndrome (ALGS) in a subject in need thereof, the method comprising administering (e.g., orally) to the subject a therapeutically effective amount of a pharmaceutical formulation comprising an IBAT inhibitor, or a pharmaceutically acceptable salt thereof. Also provided herein are methods for treating pruritus associated with Alagille syndrome (ALGS) in a subject in need thereof, the method comprising administering (e.g., orally) to the subject a therapeutically effective amount of a pharmaceutical formulation comprising an IBAT inhibitor, or a pharmaceutically acceptable salt thereof. In some embodiments, provided herein are methods for treating cholestasis associated with ALGS in a subject in need thereof, the method comprising administering (e.g., orally) to the subject a therapeutically effective amount of a pharmaceutical formulation comprising an IBAT inhibitor, or a pharmaceutically acceptable salt thereof

In some embodiments, following administration of the IBAT inhibitor, the subject exhibits a reduction in mean monthly pruritus score.

In some embodiments, the reduction in mean monthly pruritus score (i.e., scratching score) is at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, at least 3.4, at least 3.5, at least 3.6, at least 3.7, at least 3.8, or at least 3.9. In some embodiments, the reduction in mean monthly pruritus score is about 0.3 to about 4.0. For example, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.5 to about 3.5, about 0.5 to about 3.0, about 0.5 to about 2.5, about 0.5 to about 2.0, about 0.5 to about 1.5, about 0.5 to about 1.0, about 1.0 to about 4.0 about 1.0 to about 3.5, about 1.0 to about 3.0, about 1.0 to about 2.5, about 1.0 to about 2.0, about 1.0 to about 1.5, about 1.5 to about 4.0, about 1.5 to about 3.5, about 1.5 to about 3.0, about 1.5 to about 2.5, about 1.5 to about 2.0, about 2.0 to about 1.0, about 2.0 to about 3.5, about 2.0 to about 3.0, about 2.0 to about 2.5, about 2.5 to about 4.0 about 2.5 to about 3.5, about 2.5 to about 3.0, about 3.0 to about 4.0, about 3.0 to about 3.5, or about 3.5 to about 4.0. In some embodiments, the reduction in mean monthly pruritus score is about 0.5 to about 2.0 (e.g., about 0.5 to about 1.5; about 0.5 to about 1.2; about 0.8 to about 1.4; about 0.9 to about 1.2; about 1.2 to about 2.0; about 1.2 to about 1.5; about 1.2 to about 1.8; about 1.4 to about 2.0; about 1.6 to about 2.0; about 1.5 to about 2.0; about 1.3 to about 1.6; or about 1.4 to about 1.8). In some embodiments, the reduction in mean monthly pruritus score is about 2.0. In some embodiments, the reduction in mean monthly pruritus score is about 1.6.

In some embodiments, the reduction in mean monthly pruritus score occurs after about 1 week to about 72 weeks of administration of the IBAT inhibitor. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of the MAT inhibitor. In some embodiments, the reduction in mean monthly pruritus score occurs after about 5 weeks to about 8 weeks, about 9 to about 12 weeks, about 21 to about 24 weeks of administration of the IBAT inhibitor.

In some embodiments, the reduction in mean monthly pruritus score occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of the IBAT inhibitor.

In some embodiments, the reduction in mean monthly pruritus score occurs following administration of the IBAT inhibitor for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the reduction in mean monthly pruritus score occurs following administration of the IBAT inhibitor for at least 4 weeks, at least 8 weeks, at least 12 weeks, at least 16 weeks, at least 20 weeks, or at least 24 weeks. In some embodiments, the reduction in mean monthly pruritus score occurs following 4 weeks of administration. In some embodiments, the reduction in mean monthly pruritus score occurs following 24 weeks of administration. In some embodiments, the reduction in mean monthly pruritus score occurs following 48 weeks of administration. In some embodiments, the reduction in mean monthly pruritus score occurs following 72 weeks of administration.

In some embodiments, the reduction in mean monthly pruritus score is about 0.3 to about 2.0 following 24 weeks of administration of the IBAT inhibitor. In some embodiments, the reduction in mean monthly pruritus score is about 0.5 to about 1.5 following 24 weeks of administration of the IBAT inhibitor. For example, the reduction in mean monthly pruritus score is about 0.9 to about 1.3 following 24 weeks of administration of the IBAT inhibitor. In some embodiments, the reduction in mean monthly pruritus score is about 1.1 following 24 weeks of administration of the IBAT inhibitor.

In some embodiments, the reduction in mean monthly pruritus score is about 1.2 to about 2.0 following 48 weeks of administration of the IBAT inhibitor. For example, the reduction in mean monthly pruritus score is about 1.4 to about 1.8 following 48 weeks of administration of the IBAT inhibitor. In some embodiments, the reduction in mean monthly pruritus score is about 1.6 following 48 weeks of administration of the IBAT inhibitor.

In some embodiments, following administration of the IBAT inhibitor, the subject exhibits a reduction in pruritus score relative to baseline. In some embodiments, the reduction in pruritus score (i.e., scratching score) relative to baseline is at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, at least 3.4, at least 3.5, at least 3.6, at least 3.7, at least 3.8, or at least 3.9. In some embodiments, the reduction in pruritus score relative to baseline is about 0.3 to about 4.0. For example, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.5 to about 3.5, about 0.5 to about 3.0, about 0.5 to about 2.5, about 0.5 to about 2.0, about 0.5 to about 1.5, about 0.5 to about 1.0, about 1.0 to about 4.0 about 1.0 to about 3.5, about 1.0 to about 3.0, about 1.0 to about 2.5, about 1.0 to about 2.0, about 1.0 to about 1.5, about 1.5 to about 4.0, about 1.5 to about 3.5, about 1.5 to about 3.0, about 1.5 to about 2.5, about 1.5 to about 2.0, about 2.0 to about 1.0, about 2.0 to about 3.5, about 2.0 to about 3.0, about 2.0 to about 2.5, about 2.5 to about 4.0 about 2.5 to about 3.5, about 2.5 to about 3.0, about 3.0 to about 4.0, about 3.0 to about 3.5, or about 3.5 to about 4.0. In some embodiments, the reduction in pruritus score relative to baseline is about 0.5 to about 2.0 (e.g., about 0.5 to about 1.5; about 0.5 to about 1.2; about 0.8 to about 1.4; about 0.9 to about 1.2; about 1.2 to about 2.0; about 1.2 to about 1.5; about 1.2 to about 1.8; about 1.4 to about 2.0; about 1.6 to about 2.0; about 1.5 to about 2.0; about 1.3 to about 1.6; or about 1.4 to about 1.8). In some embodiments, the reduction in pruritus score relative to baseline is about 2.0. In some embodiments, the reduction in pruritus score relative to baseline is about 1.6.

In some embodiments, the reduction in pruritus score relative to baseline occurs after about 1 week to about 72 weeks of administration of the IBAT inhibitor. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of the IBAT inhibitor. In some embodiments, the reduction in pruritus score relative to baseline occurs after about 5 weeks to about 8 weeks, about 9 to about 12 weeks, about 13 to about 16 weeks, about 17 to about 20 weeks, about 21 to about 24 weeks of administration of the IBAT inhibitor.

In some embodiments, the reduction in pruritus score relative to baseline occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of the IBAT inhibitor.

In some embodiments, the reduction in pruritus score relative to baseline occurs following administration of the IBAT inhibitor, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the reduction in pruritus score relative to baseline occurs following administration of the IBAT inhibitor, for at least 4 weeks, at least 8 weeks, at least 12 weeks, at least 16 weeks, at least 20 weeks, or at least 24 weeks. In some embodiments, the reduction in pruritus score relative to baseline occurs following 1 week of administration. In some embodiments, the reduction in pruritus score relative to baseline occurs following 4 weeks of administration. In some embodiments, the reduction in pruritus score relative to baseline occurs following 24 weeks of administration. In some embodiments, the reduction in pruritus score relative to baseline occurs following 48 weeks of administration. In some embodiments, the reduction in pruritus score relative to baseline occurs following 72 weeks of administration.

In some embodiments, the reduction in pruritus score relative to baseline is about 0.3 to about 2.0 following 24 weeks of administration of the IBAT inhibitor. In some embodiments, the reduction in pruritus score relative to baseline is about 0.5 to about 1.5 following 24 weeks of administration of the IBAT inhibitor. For example, the reduction in pruritus score relative to baseline is about 0.9 to about 1.3 following 24 weeks of administration of the IBAT inhibitor. In some embodiments, the reduction in pruritus score relative to baseline is about 1.1 following 24 weeks of administration of the IBAT inhibitor.

In some embodiments, the reduction in pruritus score relative to baseline is about 1.2 to about 2.0 following 48 weeks of administration of the IBAT inhibitor. For example, the reduction in pruritus score relative to baseline is about 1.4 to about 1.8 following 48 weeks of administration of the IBAT inhibitor. In some embodiments, the reduction in pruritus score relative to baseline is about 1.6 following 48 weeks of administration of the IBAT inhibitor.

In some embodiments, the reduction is pruritus score (i.e., scratching score) relative to baseline is a reduction in: the average AM scratching score, the average PM scratching score, or average AM and PM scratching score. In some embodiments, following administration of the IBAT inhibitor, the subject exhibits a reduction in the average AM and PM scratching score relative to baseline. In some embodiments, the average AM and PM scratching score is the worst scratching score as measured by the ObsRO instrument. In some embodiments, the average AM and PM scratching score is the worst scratching score as measured by the ObsRO AM and PM caregiver reported instrument.

In some embodiments, the reduction in the average AM scratching score, the average PM scratching score, or average AM and PM scratching score relative to baseline is at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, at least 3.4, at least 3.5, at least 3.6, at least 3.7, at least 3.8, or at least 3.9. In some embodiments, the reduction in the average AM scratching score, the average PM scratching score, or average AM and PM scratching score relative to baseline is about 0.3 to about 4.0. For example, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.5 to about 3.5, about 0.5 to about 3.0, about 0.5 to about 2.5, about 0.5 to about 2.0, about 0.5 to about 1.5, about 0.5 to about 1.0, about 1.0 to about 4.0 about 1.0 to about 3.5, about 1.0 to about 3.0, about 1.0 to about 2.5, about 1.0 to about 2.0, about 1.0 to about 1.5, about 1.5 to about 4.0, about 1.5 to about 3.5, about 1.5 to about 3.0, about 1.5 to about 2.5, about 1.5 to about 2.0, about 2.0 to about 1.0, about 2.0 to about 3.5, about 2.0 to about 3.0, about 2.0 to about 2.5, about 2.5 to about 4.0 about 2.5 to about 3.5, about 2.5 to about 3.0, about 3.0 to about 4.0, about 3.0 to about 3.5, or about 3.5 to about 4.0. In some embodiments, the reduction in the average AM scratching score, the average PM scratching score, or average AM and PM scratching score relative to baseline is about 0.5 to about 2.0 (e.g., about 0.5 to about 1.5; about 0.5 to about 1.2; about 0.8 to about 1.4; about 0.9 to about 1.2; about 1.2 to about 2.0; about 1.2 to about 1.5; about 1.2 to about 1.8; about 1.4 to about 2.0; about 1.6 to about 2.0; about 1.5 to about 2.0; about 1.3 to about 1.6; or about 1.4 to about 1.8). In some embodiments, the reduction in the average AM scratching score, the average PM scratching score, or average AM and PM scratching score relative to baseline is about 1.1. In some embodiments, reduction in the average AM scratching score, the average PM scratching score, or average AM and PM scratching score relative to baseline is about 1.6. In some embodiments, the reduction in the average AM scratching score, the average PM scratching score, or average AM and PM scratching score relative to baseline is about 2.0.

In some embodiments, reduction in the average AM scratching score, the average PM scratching score, or average AM and PM scratching score relative to baseline occurs after about 1 week to about 72 weeks of administration of the IBAT inhibitor. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 1 week to about 72 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of the IBAT inhibitor. In some embodiments, the reduction in the average AM scratching score, the average PM scratching score, or average AM and PM scratching score relative to baseline occurs after about 5 weeks to about 8 weeks, about 9 to about 12 weeks, about 21 to about 24 weeks of administration of the IBAT inhibitor.

In some embodiments, the reduction in the average AM scratching score, the average PM scratching score, or average AM and PM scratching score relative to baseline occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of the IBAT inhibitor.

In some embodiments, the reduction in the average AM scratching score, the average PM scratching score, or average AM and PM scratching score relative to baseline occurs following administration of the IBAT inhibitor, for at least 1 week, at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, reduction in the average AM scratching score, the average PM scratching score, or average AM and PM scratching score relative to baseline occurs following administration of the IBAT inhibitor, for at least 1 week, at least 4 weeks, at least 8 weeks, at least 12 weeks, at least 16 weeks, at least 20 weeks, or at least 24 weeks.

In some embodiments, the subject exhibits a reduction in mean serum bile acid concentration.

In some embodiments, the reduction from baseline in mean serum bile acid concentration is at least 25 μmol/L, at least 50 μmol/L, at least 75 μmol/L, at least 100 μmol/L, at least 125 μmol/L, at least 150 μmol/L, at least 175 μmol/L, at least 200 μmol/L, at least 300 μmol/L, at least 400 μmol/L, at least 500 μmol/L, or at least 600 μmol/L relative to baseline. For example, the reduction in mean serum bile acid concentration is about 25 μmol/L to about 200 μmol/L relative to baseline (e.g., about 25 μmol/L to about 50 μmol/L; about 25 μmol/L to about 50 μmol/L; about 25 μmol/L to about 75 μmol/L; about 25 μmol/L to about 100 μmol/L; about 25 μmol/L to about 125 μmol/L; about 25 μmol/L to about 150 μmol/L; about 25 μmol/L to about 175 μmol/L; about 25 μmol/L to about 75 μmol/L). In some embodiments, the reduction in mean serum bile acid concentration is about 50 μmol/L to about 180 μmol/L relative to baseline (e.g., about 50 μmol/L to about 100 μmol/L; about 50 μmol/L to about 120 μmol/L; about 50 μmol/L to about 150 μmol/L; about 65 μmol/L to about 120 μmol/L; about 50 μmol/L to about 90 μmol/L; about 65 μmol/L to about 85 μmol/L; about 100 μmol/L to about 130 μmol/L; about 100 μmol/L to about 180 μmol/L; or about 150 μmol/L to about 180 μmol/L). In some embodiments, the reduction in mean serum bile acid concentration is about 50 μmol/L to about 600 μmol/L, about 100 μmol/L to about 600 μmol/L, about 150 μmol/L to about 600 μmol/L, about 200 μmol/L to about 600 μmol/L, about 250 μmol/L to about 600 μmol/L, about 300 μmol/L to about 600 μmol/L, about 350 μmol/L to about 600 μmol/L, about 400 μmol/L to about 600 μmol/L, about 450 μmol/L to about 600 μmol/L, about 500 μmol/L to about 600 μmol/L, about 550 μmol/L to about 600 μmol/L, about 50 μmol/L to about 500 μmol/L, about 100 μmol/L to about 500 μmol/L, about 150 μmol/L to about 500 μmol/L, about 200 μmol/L to about 500 μmol/L, about 250 μmol/L to about 500 μmol/L, about 300 μmol/L to about 500 μmol/L, about 350 μmol/L to about 500 μmol/L, about 400 μmol/L to about 500 μmol/L, about 450 μmol/L to about 500 μmol/L, about 50 μmol/L to about 400 μmol/L, about 100 μmol/L to about 400 μmol/L, about 150 μmol/L to about 400 μmol/L, about 200 μmol/L to about 400 μmol/L, about 250 μmol/L to about 400 μmol/L, about 300 μmol/L to about 400 μmol/L, about 350 μmol/L to about 400 μmol/L, about 50 μmol/L to about 300 μmol/L, about 100 μmol/L to about 300 μmol/L, about 150 μmol/L to about 300 μmol/L, about 200 μmol/L to about 300 μmol/L, about 250 μmol/L to about 300 μmol/L, about 50 μmol/L to about 300 μmol/L, about 100 μmol/L to about 300 μmol/L, about 150 μmol/L to about 300 μmol/L, about 200 μmol/L to about 300 μmol/L, about 250 μmol/L to about 300 μmol/L, about 50 μmol/L to about 200 μmol/L, about 100 μmol/L to about 200 μmol/L, or about 150 μmol/L to about 200 μmol/L relative to baseline. In some embodiments, the reduction in mean serum bile acid concentration of about 70 μmol/L to about 120 μmol/L relative to baseline. In some embodiments, the reduction in mean serum bile acid concentration is about 150 μmol/L to about 180 μmol/L.

In some embodiments, the reduction in mean serum bile acid concentration occurs after about 1 week to about 72 weeks of administration of the IBAT inhibitor. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of the IBAT inhibitor. In some embodiments, the reduction in mean serum bile acid concentration occurs after about 5 weeks to about 8 weeks, about 9 to about 12 weeks, about 21 to about 24 weeks of administration of the IBAT inhibitor.

In some embodiments, the reduction in mean serum bile acid concentration occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of the IBAT inhibitor.

In some embodiments, the reduction in mean serum bile acid concentration occurs following administration of the IBAT inhibitor, for at least 1 week, at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 60 weeks, least 72 weeks, at least 96 weeks, etc. In some embodiments, the reduction in mean serum bile acid concentration occurs following 4 weeks of administration. In some embodiments, the reduction in mean serum bile acid concentration occurs following 12 weeks of administration. In some embodiments, the reduction in mean serum bile acid concentration occurs following 24 weeks of administration. In some embodiments, the reduction in mean serum bile acid concentration occurs following 48 weeks of administration. In some embodiments, the reduction in mean serum bile acid concentration occurs following 72 weeks of administration.

In some embodiments, the reduction in mean serum bile acid concentration is about 50 μmol/L to about 90 μmol/L following 12 weeks of administration of the IBAT inhibitor. For example, the reduction in mean serum bile acid concentration is about 65 μmol/L to about 85 μmol/L following 12 weeks of administration of the IBAT inhibitor. In some embodiments, the reduction in mean serum bile acid concentration is about 70 μmol/L (e.g., about 73 μmol/L) following 12 weeks of administration of the IBAT inhibitor.

In some embodiments, the reduction in mean serum bile acid concentration is about 100 μmol/L to about 130 μmol/L following 24 weeks of administration of the IBAT inhibitor. For example, the reduction in mean serum bile acid concentration is about 110 μmol/L to about 120 μmol/L following 24 weeks of administration of the IBAT inhibitor. In some embodiments, the reduction in mean serum bile acid concentration is about 115 following 24 weeks of administration of the IBAT inhibitor.

In some embodiments, the reduction in mean serum bile acid concentration is about 150 μmol/L to about 180 μmol/L following 48 weeks of administration of the IBAT inhibitor. For example, the reduction in mean serum bile acid concentration is about 155 μmol/L to about 170 μmol/L following 48 weeks of administration of the IBAT inhibitor. In some embodiments, the reduction in mean serum bile acid concentration is about 165 μmol/L (e.g., about 166 μmol/L) following 48 weeks of administration of the IBAT inhibitor.

In some embodiments, the subject exhibits a reduction in serum bile acid concentration relative to baseline. In some embodiments, the reduction in serum bile acid concentration is at least 25 μmol/L, at least 50 μmol/L, at least 75 μmol/L, at least 100 μmol/L, at least 125 μmol/L, at least 150 μmol/L, at least 175 μmol/L, at least 200 μmol/L, at least 300 μmol/L, at least 400 μmol/L, at least 500 μmol/L, or at least 600 μmol/L relative to baseline. For example, the reduction in mean serum bile acid concentration is about 25 μmol/L to about 200 μmol/L relative to baseline (e.g., about 25 μmol/L to about 50 μmol/L; about 25 μmol/L to about 50 μmol/L; about 25 μmol/L to about 75 μmol/L; about 25 μmol/L to about 100 μmol/L; about 25 μmol/L to about 125 μmol/L; about 25 μmol/L to about 150 μmol/L; about 25 μmol/L to about 175 μmol/L; about 25 μmol/L to about 75 μmol/L). In some embodiments, the reduction in serum bile acid concentration is about 50 μmol/L to about 180 μmol/L relative to baseline (e.g., about 50 μmol/L to about 100 μmol/L; about 50 μmol/L to about 120 μmol/L; about 50 μmol/L to about 150 μmol/L; about 65 μmol/L to about 120 μmol/L; about 50 μmol/L to about 90 μmol/L; about 65 μmol/L to about 85 μmol/L; about 100 μmol/L to about 130 μmol/L; about 100 μmol/L to about 180 μmol/L; or about 150 μmol/L to about 180 μmol/L). In some embodiments, the reduction in serum bile acid concentration is about 50 μmol/L to about 600 μmol/L, about 100 μmol/L to about 600 μmol/L, about 150 μmol/L to about 600 μmol/L, about 200 μmol/L to about 600 μmol/L, about 250 μmol/L to about 600 μmol/L, about 300 μmol/L to about 600 μmol/L, about 350 μmol/L to about 600 μmol/L, about 400 μmol/L to about 600 μmol/L, about 450 μmol/L to about 600 μmol/L, about 500 μmol/L to about 600 μmol/L, about 550 μmol/L to about 600 μmol/L, about 50 μmol/L to about 500 μmol/L, about 100 μmol/L to about 500 μmol/L, about 150 μmol/L to about 500 μmol/L, about 200 μmol/L to about 500 μmol/L, about 250 μmol/L to about 500 μmol/L, about 300 μmol/L to about 500 μmol/L, about 350 μmol/L to about 500 μmol/L, about 400 μmol/L to about 500 μmol/L, about 450 μmol/L to about 500 μmol/L, about 50 μmol/L to about 400 μmol/L, about 100 μmol/L to about 400 μmol/L, about 150 μmol/L to about 400 μmol/L, about 200 μmol/L to about 400 μmol/L, about 250 μmol/L to about 400 μmol/L, about 300 μmol/L to about 400 μmol/L, about 350 μmol/L to about 400 μmol/L, about 50 μmol/L to about 300 μmol/L, about 100 μmol/L to about 300 μmol/L, about 150 μmol/L to about 300 μmol/L, about 200 μmol/L to about 300 μmol/L, about 250 μmol/L to about 300 μmol/L, about 50 μmol/L to about 300 μmol/L, about 100 μmol/L to about 300 μmol/L, about 150 μmol/L to about 300 μmol/L, about 200 μmol/L to about 300 μmol/L, about 250 μmol/L to about 300 μmol/L, about 50 μmol/L to about 200 μmol/L, about 100 μmol/L to about 200 μmol/L, or about 150 μmol/L to about 200 μmol/L relative to baseline. In some embodiments, the reduction in serum bile acid concentration of about 70 μmol/L to about 120 μmol/L relative to baseline. In some embodiments, the reduction in serum bile acid concentration is about 150 μmol/L to about 180 μmol/L.

In some embodiments, the reduction in serum bile acid concentration occurs after about 1 weeks to about 72 weeks of administration of the IBAT inhibitor. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of the IBAT inhibitor. In some embodiments, the reduction in serum bile acid concentration occurs after about 5 weeks to about 8 weeks, about 9 to about 12 weeks, about 21 to about 24 weeks of administration of the IBAT inhibitor.

In some embodiments, the reduction in serum bile acid concentration occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of the IBAT inhibitor.

In some embodiments, the reduction in serum bile acid concentration occurs following administration of the IBAT inhibitor, for at least 1 week, at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 54 weeks, at least 60 weeks, at least 66 weeks, at least 72 weeks, etc. In some embodiments, the reduction in serum bile acid concentration occurs following 4 weeks of administration. In some embodiments, the reduction in serum bile acid concentration occurs following 12 weeks of administration. In some embodiments, the reduction in serum bile acid concentration occurs following 24 weeks of administration. In some embodiments, the reduction in serum bile acid concentration occurs following 48 weeks of administration. In some embodiments, the reduction in serum bile acid concentration occurs following 72 weeks of administration.

In some embodiments, the reduction in serum bile acid concentration is about 50 μmol/L to about 90 μmol/L following 12 weeks of administration of the IBAT inhibitor. For example, the reduction in serum bile acid concentration is about 65 μmol/L to about 85 μmol/L following 12 weeks of administration of the IBAT inhibitor. In some embodiments, the reduction in serum bile acid concentration is about 70 μmol/L (e.g., about 73 μmol/L) following 12 weeks of administration of the IBAT inhibitor. In some embodiments, the reduction in serum bile acid concentration is about 100 μmol/L to about 130 μmol/L following 24 weeks of administration of the IBAT inhibitor. For example, the reduction in serum bile acid concentration is about 110 μmol/L to about 120 μmol/L following 24 weeks of administration of the IBAT inhibitor. In some embodiments, the reduction in serum bile acid concentration is about 115 μmol/L following 24 weeks of administration of the IBAT inhibitor.

In some embodiments, the reduction in serum bile acid concentration is about 150 μmol/L to about 180 μmol/L following 48 weeks of administration of the IBAT inhibitor. For example, the reduction in serum bile acid concentration is about 155 μmol/L to about 170 μmol/L following 48 weeks of administration of the IBAT inhibitor. In some embodiments, the reduction in serum bile acid concentration is about 165 μmol/L (e.g., about 166 μmol/L) following 48 weeks of administration of the IBAT inhibitor.

In some embodiments, following administration of the IBAT inhibitor, for at least 24 weeks, the subject exhibits a serum bile acid concentration of less than 70 μmol/L (e.g., less than 60 μmol/L; less than 50 μmol/L, etc.).

In some embodiments, following administration of the IBAT inhibitor, for at least 24 weeks, the subject exhibits a reduction in serum bile acid concentration of at least 50% relative to baseline (e.g., at least 55%; at least 60; at least 65%; at least 70%; at least 75%; at least 80%; at least 85%; at least 90%; at least 95%). In some embodiments, the subject exhibits a reduction in serum bile acid concentration of at least 60%, at least 70%, or at least 80% relative to baseline.

In some embodiments, following administration of the IBAT inhibitor, the subject exhibits an increase in mean height Z score relative to baseline.

In some embodiments, following administration of the IBAT inhibitor, growth is improved relative to placebo. In some embodiments, following administration of the IBAT inhibitor, the subject exhibits an increase in mean height Z score relative to baseline. In some embodiments, the increase in mean height Z score is at least 0.1, at least 0.2, at least 0.5, at least 0.75, at least 1, at least 1.25, or at least 1.5 relative to baseline. For example, the mean height Z score increased about 0.5 to about 2.0 (e.g., about 0.5 to about 0.8; about 0.5 to about 1.2; about 0.5 to about 1.5; about 0.7 to about 1.5; about 0.8 to about 1.4; about 0.9 to about 1.3; or about 1.0 to about 1.2). In some embodiments, the mean height Z score increased about 1.1.

In some embodiments, the increase in mean height Z score occurs after about 20 week to about 72 weeks of administration of the IBAT inhibitor. For example, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of the IBAT inhibitor. In some embodiments, the increase in mean height Z score occurs after about 21 to about 24 weeks of administration of the IBAT inhibitor.

In some embodiments, the increase in mean height Z score occurs after about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of the IBAT inhibitor.

In some embodiments, the increase in mean height Z score occurs following administration of the IBAT inhibitor, for at least 20 weeks, at least 24 weeks, at least 28 weeks, at least 32 weeks, at least 36 weeks, at least 40 weeks, at least 44 weeks, at least 48 weeks, etc. In some embodiments, the increase in mean height Z score occurs following 24 weeks of administration. In some embodiments, the increase in mean height Z score occurs following 48 weeks of administration. In some embodiments, the increase in mean height Z score occurs following 72 weeks of administration.

In some embodiments, the mean height Z score increases about 0.9 to about 1.3 following administration of the IBAT inhibitor, for 48 weeks. In some embodiments, the mean height Z score increases about 1.0 to about 1.2 following administration of the IBAT inhibitor, for 48 weeks. In some embodiments, the mean height Z score increases about 1.1 following administration of the IBAT inhibitor, for 48 weeks.

In some embodiments, following administration of the IBAT inhibitor, the subject exhibits an increase in mean weight Z score.

In some embodiments, the increase in mean weight Z score is at least 0.2, at least 0.4, at least 0.6, at least 0.8, at least 1, at least 1.2, or at least 1.4. For example, the mean weight Z score increased about 0.2 to about 1.5 (e.g., about 0.5 to about 0.8; about 0.5 to about 1.2; about 0.5 to about 1.5; about 0.7 to about 1.5; about 0.8 to about 1.4; about 0.9 to about 1.3; or about 1.0 to about 1.2). In some embodiments, the mean weight Z score increased about 1.1.

In some embodiments, the increase in mean weight Z score occurs after about 1 week to about 72 weeks of administration of the MAT inhibitor. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of the IBAT inhibitor. In some embodiments, the increase in mean weight Z score occurs after about 5 weeks to about 8 weeks, about 9 to about 12 weeks, about 21 to about 24 weeks of administration of the IBAT inhibitor.

In some embodiments, the increase in mean weight Z score occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of the IBAT inhibitor.

In some embodiments, the increase in mean weight Z score occurs following administration of the IBAT inhibitor, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the increase in mean weight Z score occurs following administration of the IBAT inhibitor, for at least 4 weeks, at least 8 weeks, at least 12 weeks, at least 16 weeks, at least 20 weeks, or at least 24 weeks. In some embodiments, the increase in mean weight Z score occurs following 12 weeks of administration. In some embodiments, the increase in mean weight Z score occurs following 24 weeks of administration. In some embodiments, the increase in mean weight Z score occurs following 48 weeks of administration. In some embodiments, the increase in mean weight Z score occurs following 72 weeks of administration.

In some embodiments, the mean weight Z score increases about 0.9 to about 1.3 following administration of the IBAT inhibitor, for 48 weeks. In some embodiments, the mean weight Z score increases about 1.0 to about 1.2 following administration of the IBAT inhibitor, for 48 weeks. In some embodiments, the mean weight Z score increases about 1.1 following administration of the IBAT inhibitor, for 48 weeks.

In some embodiments, following administration of the IBAT inhibitor, the subject exhibits an increase in height Z score relative to baseline. In some embodiments, the increase in height Z score is at least 0.1, at least 0.2, at least 0.5, at least 0.75, at least 1, at least 1.25, or at least 1.5 relative to baseline. For example, the height Z score increased about 0.5 to about 2.0 (e.g., about 0.5 to about 0.8; about 0.5 to about 1.2; about 0.5 to about 1.5; about 0.7 to about 1.5; about 0.8 to about 1.4; about 0.9 to about 1.3; or about 1.0 to about 1.2). In some embodiments, the height Z score increased about 1.1.

In some embodiments, the increase in height Z score occurs after about 20 week to about 72 weeks of administration of the IBAT inhibitor. For example, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of the IBAT inhibitor. In some embodiments, the increase in height Z score occurs after about 21 to about 24 weeks of administration of the IBAT inhibitor.

In some embodiments, the increase in height Z score occurs after about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of the IBAT inhibitor.

In some embodiments, the increase in height Z score occurs following administration of the IBAT inhibitor, for at least 20 weeks, at least 24 weeks, at least 28 weeks, at least 32 weeks, at least 36 weeks, at least 40 weeks, at least 44 weeks, at least 48 weeks, etc. In some embodiments, the increase in height Z score occurs following 24 weeks of administration. In some embodiments, the increase in height Z score occurs following 48 weeks of administration. In some embodiments, the increase in height Z score occurs following 72 weeks of administration.

In some embodiments, the height Z score increases about 0.9 to about 1.3 following administration of the IBAT inhibitor, for 48 weeks. In some embodiments, the height Z score increases about 1.0 to about 1.2 following administration of the IBAT inhibitor, for 48 weeks. In some embodiments, the height Z score increases about 1.1 following administration of the IBAT inhibitor, for 48 weeks.

In some embodiments, following administration of the IBAT inhibitor, or a pharmaceutically acceptable salt thereof, the subject exhibits an increase in weight Z score.

In some embodiments, the increase in weight Z score is at least 0.2, at least 0.4, at least 0.6, at least 0.8, at least 1, at least 1.2, or at least 1.4. For example, the weight Z score increased about 0.2 to about 1.5 (e.g., about 0.5 to about 0.8; about 0.5 to about 1.2; about 0.5 to about 1.5; about 0.7 to about 1.5; about 0.8 to about 1.4; about 0.9 to about 1.3; or about 1.0 to about 1.2). In some embodiments, the weight Z score increased about 1.1.

In some embodiments, the increase in weight Z score occurs after about 1 week to about 72 weeks of administration of the IBAT inhibitor. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of the IBAT inhibitor. In some embodiments, the increase in weight Z score occurs after about 5 weeks to about 8 weeks, about 9 to about 12 weeks, about 21 to about 24 weeks of administration of the IBAT inhibitor.

In some embodiments, the increase in weight Z score occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of the IBAT inhibitor.

In some embodiments, the increase in weight Z score occurs following administration of the IBAT inhibitor, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the increase in weight Z score occurs following administration of the IBAT inhibitor, for at least 4 weeks, at least 8 weeks, at least 12 weeks, at least 16 weeks, at least 20 weeks, or at least 24 weeks. In some embodiments, the increase in weight Z score occurs following 12 weeks of administration. In some embodiments, the increase in weight Z score occurs following 24 weeks of administration. In some embodiments, the increase in weight Z score occurs following 48 weeks of administration. In some embodiments, the increase in weight Z score occurs following 72 weeks of administration.

In some embodiments, the weight Z score increases about 0.9 to about 1.3 following administration of the IBAT inhibitor, for 48 weeks. In some embodiments, the weight Z score increases about 1.0 to about 1.2 following administration of the IBAT inhibitor, for 48 weeks. In some embodiments, the weight Z score increases about 1.1 following administration of the IBAT inhibitor, for 48 weeks.

In some embodiments, the subject exhibits improvement in sleep parameters following administration of the IBAT inhibitor. Improvements in sleep parameters can include, for example, mean decreases in caregiver-reported percentage of days with scratching associated with bleeding, needing help falling asleep, needing soothing, sleeping with caregiver, or taking medication to induce sleep, as well as caregiver-reported percentage of days with daytime tiredness, and caregiver-reported number of awakenings per night. As described in the Examples, at week 24, most sleep parameters for patients were significantly improved since starting the IBAT inhibitor.

In some embodiments, the subject exhibits improvement in liver parameters or liver biomarkers following administration of the IBAT inhibitor. For example, in some embodiments, levels of autotaxin, which is linked to cholestatic pruritus intensity, and/or plasma 7α-hydroxy-4-cholesten-3-one (p-C4), a marker of bile acid synthesis, are improved following administration of the IBAT inhibitor, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc.

In some embodiments, serum ALT levels are improved following administration of the IBAT inhibitor, or a pharmaceutically acceptable salt thereof. In some embodiments, serum total bilirubin levels are decreased following administration of the IBAT inhibitor, or a pharmaceutically acceptable salt thereof. In some embodiments, serum AST levels are improved following administration of the IBAT inhibitor, or a pharmaceutically acceptable salt thereof.

In some embodiments, following administration of the IBAT inhibitor, the subject exhibits a reduction in serum cholesterol levels (mmol/L).

In some embodiments, the reduction in cholesterol level is at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, or at least 2.2 mmol/L. For example, the reduction in cholesterol level is about 0.2 to about 2.5 mmol/L (e.g., about 0.2 to about 2.0; about 0.2 to about 1.5; about 0.2 to about 1.2; about 0.2 to about 1.0; about 0.2 to about 0.8; about 0.2 to about 0.5; about 0.5 to about 2.5; about 0.5 to about 2.0; about 0.5 to about 1.5; about 0.5 to about 1.2; about 0.5 to about 1.0; about 0.5 to about 0.8; about 1.0 to about 2.5; about 1.0 to about 2.0; about 1.0 to about 1.5; about 1.0 to about 1.2; about 1.2 to about 2.5; about 1.2 to about 2.0; about 1.2 to about 1.5; about 1.5 to about 2.5; about 1.5 to about 2.0; or about 2.0 to about 2.5 mmol/L). In some embodiments, the reduction in cholesterol level is about 0.5 mmol/L. In some embodiments, the reduction in cholesterol level is about 0.6 mmol/L.

In some embodiments, the reduction in cholesterol level occurs after about 1 week to about 72 weeks of administration of the IBAT inhibitor. For example, about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 40 weeks, about 4 weeks to about 48 weeks, about 4 weeks to about 52 weeks, about 4 weeks to about 60 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 28 weeks, about 8 weeks to about 36 weeks, about 8 weeks to about 40 weeks, about 8 weeks to about 48 weeks, about 8 weeks to about 52 weeks, about 8 weeks to about 60 weeks, about 8 weeks to about 72 weeks, about 12 weeks to about 16 weeks, about 12 weeks to about 20 weeks, about 12 weeks to about 24 weeks, about 12 weeks to about 28 weeks, about 12 weeks to about 36 weeks, about 12 weeks to about 40 weeks, about 12 weeks to about 48 weeks, about 12 weeks to about 52 weeks, about 12 weeks to about 60 weeks, about 12 weeks to about 72 weeks, about 16 weeks to about 20 weeks, about 16 weeks to about 24 weeks, about 16 weeks to about 28 weeks, about 16 weeks to about 36 weeks, about 16 weeks to about 40 weeks, about 16 weeks to about 48 weeks, about 16 weeks to about 52 weeks, about 16 weeks to about 60 weeks, about 16 weeks to about 72 weeks, about 20 weeks to about 24 weeks, about 20 weeks to about 28 weeks, about 20 weeks to about 36 weeks, about 20 weeks to about 40 weeks, about 20 weeks to about 48 weeks, about 20 weeks to about 52 weeks, about 20 weeks to about 60 weeks, about 20 weeks to about 72 weeks, about 24 weeks to about 28 weeks, about 24 weeks to about 36 weeks, about 24 weeks to about 40 weeks, about 24 weeks to about 48 weeks, about 24 weeks to about 52 weeks, about 24 weeks to about 60 weeks, about 24 weeks to about 72 weeks, about 28 weeks to about 36 weeks, about 28 weeks to about 40 weeks, about 28 weeks to about 48 weeks, about 28 weeks to about 52 weeks, about 28 weeks to about 60 weeks, about 28 weeks to about 72 weeks, about 36 weeks to about 40 weeks, about 36 weeks to about 48 weeks, about 36 weeks to about 52 weeks, about 36 weeks to about 60 weeks, about 36 weeks to about 72 weeks, about 40 weeks to about 44 weeks, about 40 weeks to about 48 weeks, about 40 weeks to about 52 weeks, about 40 weeks to about 60 weeks, about 40 weeks to about 72 weeks, about 44 weeks to about 48 weeks, about 44 weeks to about 52 weeks, about 44 weeks to about 60 weeks, about 44 weeks to about 72 weeks, about 48 weeks to about 52 weeks, about 48 weeks to about 60 weeks, about 48 weeks to about 72 weeks, about 52 weeks to about 60 weeks, about 52 weeks to about 72 weeks, or about 60 weeks to about 72 weeks of administration of the IBAT inhibitor. In some embodiments, the reduction in cholesterol level occurs after about 5 weeks to about 8 weeks, about 9 to about 12 weeks, about 21 to about 24 weeks of administration of the IBAT inhibitor.

In some embodiments, the reduction in cholesterol level occurs after about 1 week, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks of administration of the IBAT inhibitor.

In some embodiments, the reduction in cholesterol level occurs following administration of the IBAT inhibitor, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the reduction in cholesterol level can occur following administration of the IBAT inhibitor, for at least 4 weeks, at least 8 weeks, at least 12 weeks, at least 16 weeks, at least 20 weeks, or at least 24 weeks. In some embodiments, the reduction in cholesterol level occurs following 4 weeks of administration. In some embodiments, the reduction in cholesterol level occurs following 24 weeks of administration. In some embodiments, the reduction in cholesterol level occurs following 48 weeks of administration. In some embodiments, the reduction in cholesterol level occurs following 72 weeks of administration.

In some embodiments, the reduction in cholesterol level is about 0.4 to about 1 mmol/L following 24 weeks of administration of the IBAT inhibitor. For example, the reduction in cholesterol level is about 0.5 to about 0.8 mmol/L following 24 weeks of administration of the IBAT inhibitor. In some embodiments, the reduction in cholesterol level is about 0.6 mmol/L following 24 weeks of administration of the IBAT inhibitor.

In some embodiments, the reduction in cholesterol level is about 0.5 to about 2.0 mmol/L following 48 weeks of administration of the IBAT inhibitor. For example, the reduction in cholesterol level is about 0.8 to about 1.5 mmol/L following 48 weeks of administration of the IBAT inhibitor.

In some embodiments, following administration of the IBAT inhibitor, the subject exhibits a reduction in cholesterol level relative to baseline. In some embodiments, the reduction in cholesterol level relative to baseline is at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0 mmol/L. For example, the reduction in cholesterol level relative to baseline is about 0.3 to about 2.0 mmol/L (e.g., about 0.5 to about 1.5; about 0.5 to about 1.2; about 0.5 to about 1; or about 0.5 to about 0.8 mmol/L). In some embodiments, the reduction in cholesterol level relative to baseline is about 0.5 mmol/L. In some embodiments, the reduction in cholesterol level is about 0.6 mmol/L.

In some embodiments, the reduction in cholesterol level relative to baseline occurs following administration of the IBAT inhibitor, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, the reduction in cholesterol level relative to baseline can occur following administration of the IBAT inhibitor, for at least 4 weeks, at least 8 weeks, at least 12 weeks, at least 16 weeks, at least 20 weeks, or at least 24 weeks. In some embodiments, the reduction in cholesterol level relative to baseline occurs following 4 weeks of administration. In some embodiments, the reduction in cholesterol level relative to baseline occurs following 24 weeks of administration. In some embodiments, the reduction in cholesterol level relative to baseline occurs following 48 weeks of administration. In some embodiments, the reduction in cholesterol level relative to baseline occurs following 72 weeks of administration.

In some embodiments, the reduction in cholesterol level relative to baseline is about 0.4 to about 1 mmol/L following 24 weeks of administration of the IBAT inhibitor. For example, the reduction in cholesterol level relative to baseline is about 0.5 to about 0.8 mmol/L following 24 weeks of administration of the IBAT inhibitor. In some embodiments, the reduction in cholesterol level relative to baseline is about 0.6 mmol/L following 24 weeks of administration of the IBAT inhibitor.

In some embodiments, the reduction in cholesterol level relative to baseline is about 0.5 to about 2.0 mmol/L following 48 weeks of administration of the IBAT inhibitor. For example, the reduction in cholesterol level relative to baseline is about 0.8 to about 1.5 mmol/L following 48 weeks of administration of the IBAT inhibitor.

In some embodiments, the subject is a pediatric subject. In some embodiments, the subject is not a pediatric subject

In some embodiments, the subject is administered about 20 to about 800 μg/kg/day of the IBAT inhibitor. For example, about 20 to about 600, about 20 to about 400, about 20 to about 200, about 20 to about 180, about 20 to about 160, about 20 to about 140, about 20 to about 120, about 20 to about 100, about 20 to about 80, about 20 to about 60, about 20 to about 40, about 40 to about 800, about 40 to about 600, about 40 to about 400, about 40 to about 200, about 40 to about 180, about 40 to about 160, about 40 to about 140, about 40 to about 120, about 40 to about 100, about 40 to about 80, about 40 to about 60, about 60 to about 800, about 60 to about 600, about 60 to about 400, about 60 to about 200, about 60 to about 180, about 60 to about 160, about 60 to about 140, about 60 to about 120, about 60 to about 100, about 60 to about 80, about 80 to about 800, about 80 to about 600, about 80 to about 400, about 80 to about 200, about 80 to about 180, about 80 to about 160, about 80 to about 140, about 80 to about 120, about 80 to about 100, about 100 to about 800, about 100 to about 600, about 100 to about 400, about 100 to about 200, about 100 to about 180, about 100 to about 160, about 100 to about 140, about 100 to about 120, about 120 to about 800, about 120 to about 600, about 120 to about 400, about 120 to about 200, about 120 to about 180, about 120 to about 160, about 120 to about 140, about 140 to about 800, about 140 to about 600, about 140 to about 400, about 140 to about 200, about 140 to about 180, about 140 to about 160, about 160 to about 800, about 160 to about 600, about 160 to about 400, about 160 to about 200, about 160 to about 180, about 180 to about 800, about 180 to about 600, about 180 to about 400, about 180 to about 200, about 200 to about 800, about 200 to about 600, about 200 to about 400, about 400 to about 800, about 400 to about 600, or about 600 to about 800 μg/kg/day of the IBAT inhibitor. In some embodiments, the subject is administered about 20, about 30, about 40, about 50, about 60, about 70, about 80, about 90, about 100, about 120, about 140, about 150, about 160, about 170, about 180, about 190, about 200, about 400, about 600, or about 800 μg/kg/day of the IBAT inhibitor. In some embodiments, the subject is administered 120 μg/kg/day of the IBAT inhibitor. In some embodiments, the subject is administered 800 μg/kg/day of the IBAT inhibitor.

In some embodiments, the subject is administered the IBAT inhibitor in an amount that does not exceed 6 mg per day.

In some embodiments, the IBAT inhibitor is administered as a unit dose ranging from about 1 μg to about 100 mg, such as from about 10 μg to about 10 mg, such as from about 100 μg to about 2000 μg, or such as from about 200 μg to about 1500 μg. In some embodiments, the IBAT inhibitor is administered as a unit dose ranging from about 10 μg to about 9 mg, about 10 μg to about 8 mg, about 10 μg to about 7 mg, about 10 μg to about 6 mg, about 10 μg to about 5 mg, about 10 μg to about 4 mg, about 10 μg to about 3 mg, about 10 μg to about 2 mg, about 10 μg to about 1 mg, about 10 μg to about 800 μg, about 10 μg to about 600 μg, about 10 μg to about 400 μg, about 10 μg to about 200 μg, about 10 μg to about 100 μg, about 10 μg to about 50 μg, about 50 μg to about 10 mg, about 50 μg to about 9 mg, about 50 μg to about 8 mg, about 50 μg to about 7 mg, about 50 μg to about 6 mg, about 50 μg to about 5 mg, about 50 μg to about 4 mg, about 50 μg to about 3 mg, about 50 μg to about 2 mg, about 50 μg to about 1 mg, about 50 μg to about 800 μg, about 50 μg to about 600 μg, about 50 μg to about 400 μg, about 50 μg to about 200 μg, about 50 μg to about 100 μg, about 100 μg to about 10 mg, about 100 μg to about 9 mg, about 100 μg to about 8 mg, about 100 μg to about 7 mg, about 100 μg to about 6 mg, about 100 μg to about 5 mg, about 100 μg to about 4 mg, about 100 μg to about 3 mg, about 100 μg to about 2 mg, about 100 μg to about 1 mg, about 100 μg to about 800 μg, about 100 μg to about 600 μg, about 100 μg to about 400 μg, about 100 μg to about 200 μg, about 200 μg to about 10 mg, about 200 μg to about 9 mg, about 200 μg to about 8 mg, about 200 μg to about 7 mg, about 200 μg to about 6 mg, about 200 μg to about 5 mg, about 200 μg to about 4 mg, about 200 μg to about 3 mg, about 200 μg to about 2 mg, about 200 μg to about 1 mg, about 200 μg to about 800 μg, about 200 μg to about 600 μg, about 200 μg to about 400 μg, about 200 μg to about 10 mg, about 200 μg to about 9 mg, about 400 μg to about 8 mg, about 400 μg to about 7 mg, about 400 μg to about 6 mg, about 400 μg to about 5 mg, about 400 μg to about 4 mg, about 400 μg to about 3 mg, about 400 μg to about 2 mg, about 400 μg to about 1 mg, about 400 μg to about 800 μg, about 400 μg to about 600 μg, about 600 μg to about 10 mg, about 600 μg to about 9 mg, about 600 μg to about 8 mg, about 600 μg to about 7 mg, about 600 μg to about 6 mg, about 600 μg to about 5 mg, about 600 μg to about 4 mg, about 600 μg to about 3 mg, about 600 μg to about 2 mg, about 600 μg to about 1 mg, about 600 μg to about 800 μg, about 800 μg to about 10 mg, about 800 μg to about 9 mg, about 800 μg to about 8 mg, about 800 μg to about 7 mg, about 800 μg to about 6 mg, about 800 μg to about 5 mg, about 800 μg to about 4 mg, about 800 μg to about 3 mg, about 800 μg to about 2 mg, about 800 μg to about 1 mg, about 1 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4 mg, about 1 mg to about 3 mg, about 1 mg to about 2 mg, about 2 mg to about 10 mg, about 2 mg to about 9 mg, about 2 mg to about 8 mg, about 2 mg to about 7 mg, about 2 mg to about 6 mg, about 2 mg to about 5 mg, about 2 mg to about 4 mg, about 2 mg to about 3 mg, about 3 mg to about 10 mg, about 3 mg to about 9 mg, about 3 mg to about 8 mg, about 3 mg to about 7 mg, about 3 mg to about 6 mg, about 3 mg to about 5 mg, about 3 mg to about 4 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 10 mg, about 5 mg to about 9 mg, about 5 mg to about 8 mg, about 5 mg to about 7 mg, about 5 mg to about 6 mg, about 6 mg to about 10 mg, about 6 mg to about 9 mg, about 6 mg to about 8 mg, about 6 mg to about 7 mg, about 7 mg to about 10 mg, about 7 mg to about 9 mg, about 7 mg to about 8 mg, about 8 mg to about 10 mg, about 8 mg to about 9 mg, or about 9 mg to about 10 mg, In some embodiments, the IBAT inhibitor is administered as a unit dose of about 100 μg, about 200 μg, about 300 μg, about 400 μg, about 500 μg, about 600 μg, about 700 μg, about 800 μg, about 900 μg, about 1000 μg, about 1100 μg, about 1200 μg, about 1300 μg, about 1400 μg, about 1500 μg, about 1600 μg, about 1700 μg, about 1800 μg, about 1900 μg, or about 2000 μg.

In some embodiments, the IBAT inhibitor is administered as a unit dose of about 200 μg. In some embodiments, the IBAT inhibitor is administered as a unit dose of about 400 μg. In some embodiments, the IBAT inhibitor is administered as a unit dose of about 600 μg. In some embodiments, the IBAT inhibitor is administered as a unit dose of about 1200 μg.

The frequency of administration can vary from once or twice a week to once or more times a day, such as two or three times daily. In some embodiments, the IBAT inhibitor is administered once daily. The frequency of administration can furthermore remain constant or be variable during the duration of the treatment. Several factors can influence the frequency of administration and the effective amount of the formulation that should be used for a particular treatment, such as the severity of the condition being treated, the duration of the treatment, as well as the age, weight, sex, diet and general medical condition of the patient being treated.

In some embodiments, the IBAT inhibitor is administered as a unit dose of about 200 μg per day. In some embodiments, the IBAT inhibitor is administered as a unit dose of about 400 μg per day. In some embodiments, the IBAT inhibitor is administered as a unit dose of about 600 μg per day. In some embodiments, the IBAT inhibitor is administered as a unit dose of about 1200 μg per day.

In some embodiments, the unit dose of the IBAT inhibitor does not exceed 6 mg per day.

In some embodiments, the subject was IBAT inhibitor naïve prior to the first administration of the pharmaceutical formulation comprising the IBAT inhibitor.

### Formulations

IBAT inhibitors as provided herein can be formulated as previously described. See, for example, International Publication Nos. WO 2019/245449; WO 2020/0167981; WO 2020/0167985; WO 2020/0167964; U.S. Pat. No. 10,709,755; and U.S. Application No. US 2017/0143738.

Odevixibat, for example, exhibits high potency and should be administered in low doses, such as ranging from about 40 μg/kg/day to about 120 μg/kg/day. This can correspond to doses such as 200 μg to 7200 μg in the treatment of paediatric patients that weigh about 4 kg to ≥55.5 kg. In some embodiments, this can correspond to doses as low as 200 μg to 800 μg in the treatment of paediatric patients that weigh about 4 kg to about 20 kg (e.g., infants and toddlers). It is desirable that a formulation of odevixibat can be administered to young patients in a dosage form having a small size. It is further desirable that such a formulation has good palatability, is not perceived as gritty, and is well-tolerated by infants and small children.

Multiparticulates can be administered to infants from birth if they are administered with a liquid. For children aged approximately 6 months and older (i.e., after weaning), the multiparticulates can be administered in their solid form either directly into the mouth or mixed with semi-solid food. Particle size, shape, texture, hardness, taste and dose volume (i.e., the number of particles) have been reported to be important for acceptability of multiparticulates by infants and children (Kozarewicz, Int. J. Pharm. 2014, vol. 469, pp 245-248). Various literature reviews have been conducted on the acceptability of different oral dosage forms in paediatric and older adult patients (see e.g. Liu, et al., Drugs 2014, vol. 74, pp. 1871-1889; Drumond et al., Int. J. Pharm. 2017, vol. 521, pp. 294-305; Mistry et al., J. Pharm. Pharmacol. 2017, vol. 69, pp. 361-376; Walsh et al., Int. J. Pharm. 2017, vol. 536, pp. 547-562), but the size and/or dose volume (amount) of multiparticulates investigated have not always been reported in these reviews.

Perception of grittiness can be influenced by a range of factors including particle size, quantity, and dosing vehicle (see Mishra et al., Yakugaku Zasshi 2009, vol. 129, pp. 1537-1544; Lopez et al., Eur. J. Pharm. Sci. 2016, vol. 92, pp. 156-162), as well as the hardness and shape of the particles (Tyle, Acta Psychologica 1993, vol. 84, pp. 111-118), with irregular particles being perceived as larger than round (spherical) particles of the same size (Engelen et al., J. Text. Studies 2005, vol. 36, pp. 373-386). Grittiness perception studies have shown that grittiness scores may increase with increasing size and dose of the multiparticulates, whereas grittiness scores may decrease with increasing vehicle viscosity (Lopez et al., Eur. J. Pharm. Sci. 2016, vol. 92, pp. 156-162).

Capsules can be acceptable for children from approximately 6 years of age. The swallowability of the capsules can depend upon the dosage form dimensions (i.e. the size) and the ability of the child. The size, shape, taste and after taste are important capsule attributes that can influence patient acceptability (Kozarewicz, Int. J. Pharm. 2014, vol. 469, pp 245-248). In some embodiments, the size of the capsules is kept as small as possible, and the number of capsules required per dose is kept to a minimum, e.g. not more than 1-3 capsules.

Provided herein is a multiparticulate formulation containing low doses of odevixibat. In some embodiments, the formulation is a paediatric formulation. In some embodiments, the formulation enables weight-based dosing and can be sprinkled onto food. The formulation can be designed to have a good palatability, with an optimal balance between particle size and dose volume.

Provided herein is a pharmaceutical formulation of odevixibat, comprising a plurality of particles, wherein each particle contains odevixibat, or a pharmaceutically acceptable salt thereof, in an amount of from about 0.1% w/w to about 5.0% w/w based on the total weight of the particle.

Because of the low doses in which odevixibat is to be administered, and further because of the multiparticulate form of the application, each particle of the formulation contains only a very low amount of the active ingredient. For example, the amount of odevixibat, or a pharmaceutically acceptable salt thereof, in each particle can be from about 0.2% w/w to about 3.5% w/w, for example, from about 0.3% w/w to about 3.0% w/w, from about 0.4% w/w to about 2.5% w/w, or from about 0.5% w/w to about 2.0% w/w based on the total weight of the particle. In some embodiments, each particle contains odevixibat, or a pharmaceutically acceptable salt thereof, in an amount of about 0.5% w/w based on the total weight of the particle. In another embodiment, each particle contains odevixibat, or a pharmaceutically acceptable salt thereof, in an amount of about 1.0% w/w based on the total weight of the particle. In yet another embodiment, each particle contains odevixibat, or a pharmaceutically acceptable salt thereof, in an amount of about 1.5% w/w based on the total weight of the particle.

As used herein, the term “particles” refers to small particles ranging in size from about 0.1 to about 1.5 mm. Such particles are essentially spherical, although elongated or oblong particles also might be used. The particles may e.g. be pellets, beads, microparticles, microspheres, granules or minitablets, and may optionally be coated with one or more coating layers surrounding every such pellet, bead, microparticle, microsphere, granule or minitablet.

In some embodiments, the particles of the formulation are small enough, that they can be sprinkled onto food and easily swallowed. In some embodiments, the particles can be swallowed without causing a perception of grittiness. In some embodiments, the particles do not give the patient an urge to chew the particles. The particles are, therefore, between about 0.1 and about 1.5 mm in size, for example, between about 0.1 and about 1.0 mm, or between about 0.1 and 0.8 mm, such as about 0.2 mm, about 0.3 mm, about 0.4 mm, about 0.5 mm, about 0.6 mm, or about 0.7 mm. In some embodiments, the particles are between about 0.4 and about 0.8 mm, such as about 0.5 mm, or such as about 0.6 mm, or such as about 0.7 mm. In some embodiments, the particles are about 0.7 mm.

In some embodiments, provided herein is a formulation of odevixibat, wherein each particle comprises a core and a coating layer surrounding the core. The core of each particle may be a pellet, a granule, a minitablet, a bead, a microparticle or a microsphere.

In some embodiments, the core of each particle comprises the active pharmaceutical ingredient (odevixibat), while the coating layer of each particle does not comprise the active pharmaceutical ingredient. In some embodiments, the core of each particle comprises from about 0.1% to about 5% w/w of the active pharmaceutical ingredient, based on the total weight of the particle, such as from about 0.1% to about 2% w/w, such as from about 0.1% to about 1% w/w, or such as from about 0.1% to about 0.5% w/w of the active pharmaceutical ingredient, based on the total weight of the particle.

In some embodiments, the coating layer of each particle contains the active pharmaceutical ingredient (odevixibat), while the core of each particle does not comprise the active pharmaceutical ingredient. In some embodiments, the coating layer of each particle contains from about 0.1% to about 5% w/w of the active pharmaceutical ingredient, based on the total weight of the particle, such as from about 0.1% to about 2% w/w, such as from about 0.1% to about 1% w/w, or such as from about 0.1% to about 0.5% w/w of the active pharmaceutical ingredient, based on the total weight of the particle.

The cores can be orally dispersible and contain soluble ingredients such as a sugar (e.g., sucrose) or a soluble polymer (e.g. hydroxypropyl methylcellulose) or may be non-orally dispersible and contain non-soluble ingredients such as a non-soluble polymer (e.g., microcrystalline cellulose). In some embodiments, the cores contain microcrystalline cellulose. In some embodiments, the cores are microcrystalline cellulose spheres.

The coating layer can further contain a film-forming polymer, such as a cellulose-based polymer, a polysaccharide-based polymer, an N-vinylpyrrolidone-based polymer, an acrylate, an acrylamide, or copolymers thereof. Examples of suitable film-forming polymers include polyvinyl alcohol (PVA), polyvinyl acetate phthalate (PVAP), polyethylene glycol (PEG), polyvinylpyrrolidone (PVP), methacrylic acid copolymers, starch, hydroxypropyl starch, chitosan, shellac, methyl cellulose, hydroxypropyl cellulose (HPC), low-substituted hydroxypropyl cellulose, hydroxypropyl methylcellulose (HPMC; or hypromellose), hydroxypropyl methylcellulose acetate succinate (HPMCAS), hydroxypropyl methylcellulose phthalate (HPMCP), cellulose acetate phthalate (CAP), cellulose acetate trimellitate (CAT), as well as combinations thereof, such as a mixture of methyl cellulose and hydroxypropyl methylcellulose (metolose). In some embodiments, the coating layer comprises a film-forming polymer selected from the group consisting of hydroxypropyl methylcellulose, polyvinyl alcohol (PVA), polyethylene glycol (PEG), starch, hydroxypropyl starch and hydroxypropyl cellulose (HPC). For example, the coating layer can contain hydroxypropyl methylcellulose as the film-forming polymer.

The coating layer can optionally comprise one or more additional ingredients, such as a plasticizer (e.g. polyethylene glycol, triacetin or triethyl citrate), an anti-tack agent (e.g. talc or magnesium stearate), or a colouring agent (e.g. titanium dioxide, iron oxides, riboflavin or turmeric).

In some embodiments, the formulation contains odevixibat in crystalline form. In some embodiments, the formulation contains a crystalline hydrate of odevixibat. In some embodiments, the formulation contains crystal modification 1 of odevixibat. This stable crystal modification can be obtained from a slurry of odevixibat in a mixture of water and an organic solvent such as ethanol. Under these conditions, a mixed solvate containing about two moles of water and about one to about three, such as about two to about three, moles of ethanol per mole of odevixibat (e.g., a dihydrate-diethanolate or a dihydrate-triethanolate) is initially formed. This mixed solvate is referred to herein as crystal modification 2. When crystal modification 2 is dried, such as under vacuum (e.g., less than 5 mbar) or under a nitrogen flow, it loses its organic solvent molecules and becomes crystal modification 1. In some embodiments, the transformation of crystal modification 2 to crystal modification 1 proceeds via a crystalline intermediate. It is believed that this crystalline intermediate is a dehydrated form, which quickly takes up water from the air. While not wishing to be bound by theory, it is believed that the solvent molecules can be removed without dissolution and recrystallization of the crystals.

Crystal modification 1 of odevixibat cannot only be obtained from a mixture of water and ethanol, as described above, but also from a slurry of odevixibat in a mixture of water and an organic solvent selected from the group consisting of methanol, 2-propanol, acetone, acetonitrile, 1,4-dioxane, DMF and DMSO. Upon drying of the different mixed solvates obtained under these conditions (crystal modification 2), the same crystalline hydrate of odevixibat is obtained, namely crystal modification 1.

Crystal modification 1 contains void volumes that are capable of containing up to about 2 moles of water associated with the crystal per mole of odevixibat, depending on the relative humidity. This form is therefore formally a channel hydrate. At about 30% relative humidity, however, crystal modification 1 contains a substantially stoichiometric amount of about 1.5 moles of water per mole of organic compound and is thus a sesquihydrate. The substantially stoichiometric amount of water is considered advantageous, as the water content of the crystals remains substantially constant even with humidity changes within the normal relative humidity range of about 30% to about 70% RH. Indeed, at normal humidities, such as between about 30 and about 70% RH, crystal modification 1 exhibits relatively low hygroscopicity.

In one embodiment, the formulation contains crystal modification 1 of odevixibat having an X-ray powder diffraction (XRPD) pattern, obtained with CuKα1-radiation, with at least specific peaks at ° 2θ positions 5.6±0.2, 6.7±0.2 and/or 12.1±0.2.

In a specific embodiment, the formulation contains crystal modification 1 having an XRPD pattern, obtained with CuKα1-radiation, with specific peaks at ° 2θ positions 5.6±0.2, 6.7±0.2 and 12.1±0.2 and one or more of the characteristic peaks: 4.1±0.2, 4.6±0.2, 9.3±0.2, 9.4±0.2 and 10.7±0.2.

In a more specific embodiment, the formulation contains crystal modification 1 having an XRPD pattern, obtained with CuKα1-radiation, with specific peaks at ° 2θ positions 4.6±0.2, 5.6±0.2, 6.7±0.2, 9.3±0.2, 9.4±0.2 and 12.1±0.2.

In a more specific embodiment, the formulation contains crystal modification 1 having an XRPD pattern, obtained with CuKα1-radiation, with characteristic peaks at ° 2θ positions 4.1±0.2, 4.6±0.2, 5.6±0.2, 6.7±0.2, 9.3±0.2, 9.4±0.2, 10.7±0.2 and 12.1±0.2, and one or more of 8.1±0.2, 8.6±0.2, 13.4±0.2, 13.8±0.2, 13.9±0.2, 16.6±0.2, 17.3±0.2, 17.7±0.2, 18.3±0.2, 18.9±0.2, 19.4±0.2, 19.7±0.2, 20.5±0.2, 20.8±0.2, 21.6±0.2, 23.2±0.2, 24.3±0.2, 29.8±0.2 and 30.6±0.2.

In an even more specific embodiment, the formulation contains crystal modification 1 having an XRPD pattern, obtained with CuKα1-radiation, with characteristic peaks at ° 2θ positions 4.1±0.2, 4.6±0.2, 5.6±0.2, 6.7±0.2, 8.1±0.2, 8.6±0.2, 9.3±0.2, 9.4±0.2, 10.7±0.2, 12.1±0.2, 13.4±0.2, 13.8±0.2, 13.9±0.2, 16.6±0.2, 17.3±0.2, 17.7±0.2, 18.3±0.2, 18.9±0.2, 19.4±0.2, 19.7±0.2, 20.5±0.2, 20.8±0.2, 21.6±0.2, 23.2±0.2, 24.3±0.2, 29.8±0.2 and 30.6±0.2.

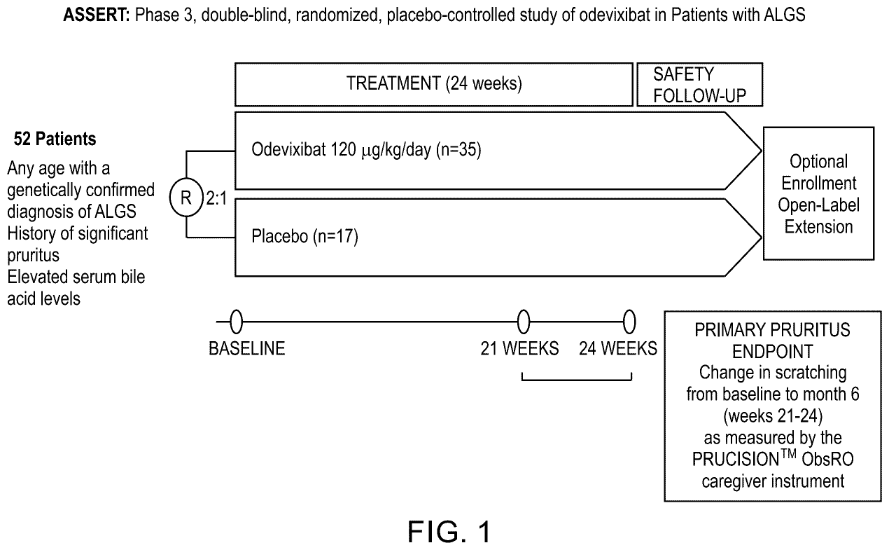

In another embodiment, the formulation contains crystal modification 1 having an XRPD pattern, obtained with CuKα1-radiation, substantially as shown in FIG. 1.

Whereas crystal modification 1 is a sesquihydrate containing about 3.5% (w/w) water at about 30% relative humidity (based on the total crystal weight), it has been observed that the crystal can take up an additional 1.5% (w/w) water when the humidity is increased up to 95% RH. The sorption and desorption of this additional water is fully reversible. The additional water may be adsorbed on the surface or may further fill the channels of the structure. In some embodiments, the term “overhydrated” refers to crystal modification 1 containing from about 1.5 to about 4 moles of water per mole of odevixibat, such as from about 1.5 to about 3.5, or such as from about 1.5 to 3, or such as from about 1.5 to about 2.5, or such as from about 1.5 to about 2 moles of water per mole of odevixibat. In some embodiments, the term “overhydrated” refers to crystal modification 1 containing from about 2 to about 4 moles of water per mole of odevixibat, such as from about 2 to about 3.5, or such as from about 2 to about 3, or such as from about 2 to 2.5 moles of water per mole of odevixibat.

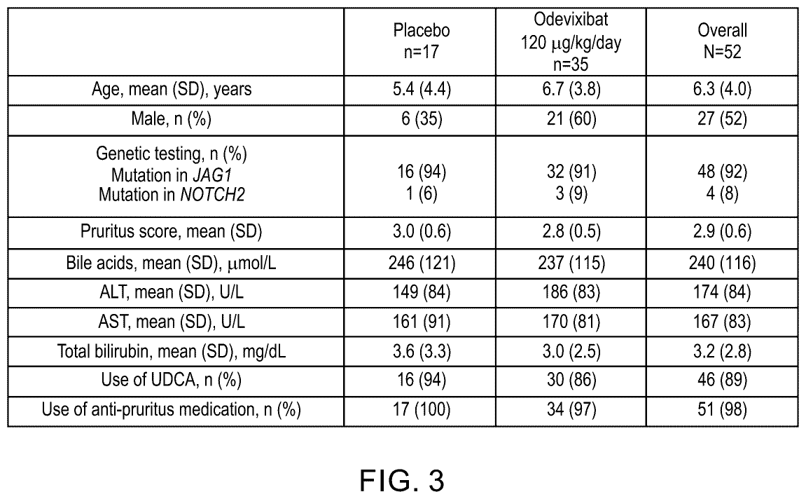

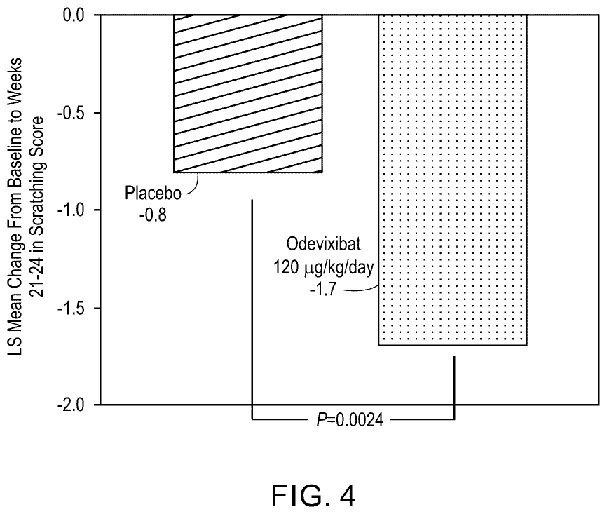

It has been observed that the XRPD pattern of overhydrated crystal modification 1 slightly changes when it is dried, e.g. at 50° C. in vacuum. A small shift of peaks is most clearly seen in the 20 ranges 5-13° and 18-25°, as shown in FIGS. 3 and 4, respectively. Exposing the dried modification to elevated relative humidity, such as up to 95% RH, makes the XRPD pattern of the overhydrated modification appear again. The peak shifts are a result of the unit cell volume changes, which occur as water molecules go in and out of the crystal structure.

Therefore, in another embodiment, the formulation contains overhydrated crystal modification 1 having an X-ray powder diffraction (XRPD) pattern, obtained with CuKα1-radiation, with at least specific peaks at ° 2θ positions 5.7±0.2, 6.7±0.2 and/or 12.0±0.2.

In a specific embodiment, the formulation contains overhydrated crystal modification 1 having an XRPD pattern, obtained with CuKα1-radiation, with specific peaks at ° 2θ positions 5.7±0.2, 6.7±0.2 and 12.0±0.2 and one or more of the characteristic peaks: 4.0±0.2, 9.4±0.2, 9.6±0.2 and 10.8±0.2.

In a more specific embodiment, the formulation contains overhydrated crystal modification 1 having an XRPD pattern, obtained with CuKα1-radiation, with specific peaks at ° 2θ positions 4.0±0.2, 5.7±0.2, 6.7±0.2, 9.4±0.2, 9.6±0.2, 10.8±0.2 and 12.1±0.2.

In a more specific embodiment, the formulation contains overhydrated crystal modification 1 having an XRPD pattern, obtained with CuKα1-radiation, with characteristic peaks at ° 2θ positions 4.0±0.2, 5.7±0.2, 6.7±0.2, 9.4±0.2, 9.6±0.2, 10.8±0.2 and 12.1±0.2, and one or more of 4.7±0.2, 8.0±0.2, 8.6±0.2, 13.3±0.2, 14.1±0.2, 15.3±0.2, 16.5±0.2, 17.3±0.2, 19.3±0.2, 19.7±0.2, 19.9±0.2, 20.1±0.2, 20.8±0.2, 21.7±0.2, 23.6±0.2, 26.2±0.2, 26.5±0.2, 28.3±0.2 and 30.9±0.2.

In an even more specific embodiment, the formulation contains overhydrated crystal modification 1 having an XRPD pattern, obtained with CuKα1-radiation, with characteristic peaks at ° 2θ positions 4.0±0.2, 4.7±0.2, 5.7±0.2, 6.7±0.2, 8.0±0.2, 8.6±0.2, 9.4±0.2, 9.6±0.2, 10.8±0.2, 12.1±0.2, 13.3±0.2, 14.1±0.2, 15.3±0.2, 16.5±0.2, 17.3±0.2, 19.3±0.2, 19.7±0.2, 19.9±0.2, 20.1±0.2, 20.8±0.2, 21.7±0.2, 23.6±0.2, 26.2±0.2, 26.5±0.2, 28.3±0.2 and 30.9±0.2.

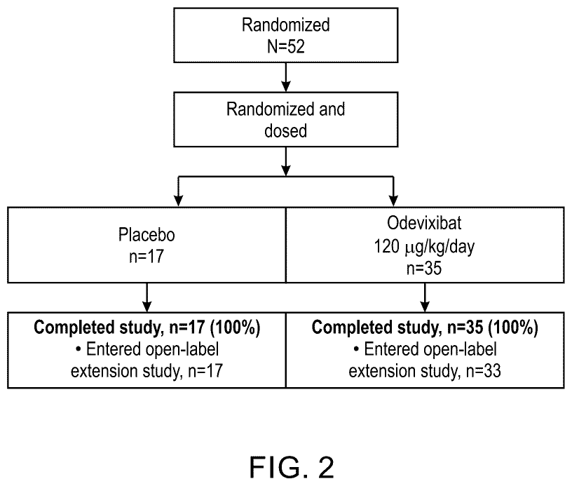

In another embodiment, the formulation contains overhydrated crystal modification 1 of odevixibat having an XRPD pattern, obtained with CuKα1-radiation, substantially as shown in FIG. 2.

It is desirable that the use of organic solvents in the preparation of the formulation is avoided. In some embodiments, water is used as the solvent for the preparation of the formulation. Odevixibat dissolves in water only very poorly, and the solubility at pH 7 and at 37° C. has been determined to be as low as about 30 μg/mL. Because of this low solubility in water, aqueous suspensions of odevixibat can contain larger agglomerates of odevixibat, which may lead to an uneven distribution of the active pharmaceutical ingredient on the cores, i.e., the cores may contain different amounts of odevixibat, which in turn impacts dose uniformity. Accordingly, in some embodiments, the aqueous suspension of odevixibat is homogeneous. In some embodiments, a homogeneous aqueous suspension of odevixibat is sprayed onto the cores.

Odevixibat exhibits high potency and it should be administered in low doses, especially in the treatment of pediatric patients that weigh about 4 to >=55.5 kg. In order to reach high dose uniformity for the multiparticulate formulation disclosed herein, it is important that each particle of the formulation substantially contains the same amount of odevixibat, i.e., the deviation in the odevixibat content of the particles of the formulation should be as low as possible.

As used herein, the term “homogeneous” refers to a suspension that does not contain agglomerates of odevixibat that are larger than about 200 μm, for example, no agglomerates larger than about 100 μm, or no agglomerates larger than about 50 μm. The size of the odevixibat agglomerates in the coating suspension may be determined by optical microscopy, using a method based on European Pharmacopoeia 9.0, monograph 2.9.37, and as described in the experimental section. Alternatively, the size of the odevixibat agglomerates in the coating suspension may be determined by light scattering techniques, such as low-angle laser light scattering (LALLS). In some embodiments, the d₉₀ value for the particle size distribution of the coating suspension is smaller than 15 μm, such as smaller than 14 μm, such as smaller than 13 μm, such as smaller than 12 μm, such as smaller than 11 μm, or such as smaller than 10 μm.

In some embodiments, a homogeneous suspension of odevixibat can be prepared by dispersing the compound in water by wet-milling. Wet-milling is a process in which a solid substance is dispersed in a liquid by shearing, by crushing, or by attrition. Examples of wet-milling apparatus include colloid mills, conical mills, ball mills, disc mills and high-shear dispersing machines. A specific example of a wet-milling apparatus for use in the formulations provided herein is a colloid mill.

In some embodiments, the crystallinity of odevixibat increases during the wet-milling.

In some embodiments, odevixibat is first wetted in a small amount of water using a homogenizer and thereafter dispersed in water using a colloid mill. Spraying the homogenized dispersion onto the cores enables an even distribution of the active pharmaceutical ingredient.

It is desirable that the formulation is free of any ingredients that are not strictly necessary for the formulation, such as surfactants. In some embodiments, therefore, the coating suspension does not contain surfactants. Similarly, in some embodiments, the coating layer of the formulation does not contain surfactants.

In one embodiment, the particles are contained within a sachet. In another embodiment, the particles are contained within a capsule. Such capsules may be made from gelatine, from a cellulose-based polymer such as a hydroxypropyl methylcellulose (hypromellose), or from a polysaccharide-based polymer such as a pullulan. Capsules may be swallowed intact, or may be designed to be opened, so that, for example, the contents (i.e. the particles) can be sprinkled onto a food vehicle for administration. In the latter case, the number of particles in one capsule should fit onto a single tablespoon of food. In some embodiments, a capsule contains from about 20 to about 100 mg of particles, such as about 30, about 40, about 50, about 60, about 70, about 80 or about 90 mg.

For younger paediatric patients, such as infants, toddlers and children up to about 6 years old, the particles can be sprinkled onto food that can be easily swallowed and which does not require chewing, such as yoghurt, applesauce, fruit purée, or oatmeal. For older paediatric patients, such as children older than about 6 years old, adolescents and younger adults, capsules containing the particles may be swallowed intact, i.e. without opening. For newborn patients up to about 6 months old, who have not yet been weaned or are unable to take semi-solid food, the formulation can be administered by dispersing the particles in a suitable liquid vehicle, such as breast milk, baby formula or water. When the particles have been dispersed in a liquid vehicle, they can be administered to the patient within 30 minutes after dispersion, without loss of the active ingredient or indications of degradation. In some embodiments, the volume of liquid vehicle used for administering the odevixibat particles, including rinsing, can be smaller than about 20 mL, such as smaller than about 15 mL, such as smaller than about 10 mL, or such as smaller than about 5 mL. In some embodiments, the dispersed particles are administered directly into the mouth using an oral syringe.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1. A Phase 3 Double-Blind, Randomized, Placebo-Controlled Study of the Safety and Efficacy of Odevixibat in Patients with Alagille Syndrome (ASSERT)

Alagille syndrome (ALGS) is a rare, multisystem disorder with a wide variety of clinical manifestations affecting the liver, heart, skeleton, eyes, central nervous system, kidneys, and facial features. It is an autosomal, dominantly-inherited disorder caused by defects in components of the NOTCH signaling pathway, most commonly due to mutations in JAG1, in about 90% of the patients. A small number of patients with ALGS have mutations in the gene for the NOTCH2 receptor. Approximately 60% of the cases represent de novo mutations. The majority of patients present early, often within the first 3 months of life, with jaundice or cardiac symptoms.

Due to the variable clinical presentations, the diagnosis of ALGS has traditionally been difficult; even the findings on histological review of liver biopsy materials may not be definitive. With the advent of genetic testing, the clinical diagnosis of ALGS is confirmed or the diagnosis itself is made by finding a mutation within the sequence analysis of JAG1 or NOTCH2.

The majority of patients with ALGS present with severe, intractable pruritus, which can be disabling. Attempts at managing pruritus are made by including ursodeoxycholic acid, cholestyramine, rifampin, ondansetron, or naltrexone and others in the patient's treatment regimen; these agents are at best partially effective. Biliary diversion surgery is occasionally used to treat intractable pruritus with some success. Treatment of persistent cholestasis and progressive liver cirrhosis is supportive and usually includes a choleretic agent. Kasai hepatoportoenterostomy (HPE) has been attempted in an effort to increase biliary flow from the liver to the intestine, but unlike patients with biliary atresia, those with ALGS who undergo the procedure have a worse outcome. Approximately 15% to 25% of patients with ALGS will require a liver transplant during childhood. For patients with ALGS there is a positive response to transplant with about 90% of patients showing improvement in liver parameters and some degree of catch-up growth. The 5-year survival post-transplant in this population is about 80%.

In addition to cholestasis and pruritus, other characteristic features of ALGS include jaundice, elevated bile acids and hepatic biochemical parameters, cardiovascular abnormalities, xanthomas, and fat-soluble vitamin deficiencies.

The IBAT, also called the apical sodium-dependent bile acid transporter (SLC10A2), is located on the luminal surface of enterocytes in the terminal ileum; this transporter mediates resorption of conjugated bile acids for recirculation back to the liver. Inhibition of IBAT disrupts the enterohepatic circulation and leads to fecal elimination of bile acids similar to surgical interruption of the enterohepatic circulation.

### Methods

### Study Design and Treatment

Odevixibat is an orally administered, potent, luminally restricted, selective IBAT inhibitor in development to treat cholestatic liver diseases. In this phase 3, double-blind, randomized, 24-week trial (ASSERT), the efficacy and safety of odevixibat versus placebo was evaluated in patients with Alagille Syndrome (ALGS). The efficacy of odevixibat in patients with ALGS was evaluated in a 24-week, randomized, double-blind, placebo-controlled trial conducted in 52 patients with a confirmed diagnosis of ALGS (NCT04674761; ASSERT). Patients were randomized 2:1 to receive odevixibat 120 μg/kg/day or placebo. The objectives of this study were to demonstrate the efficacy of repeated daily doses of 120 μg/kg/day odevixibat in relieving pruritus in patients with ALGS, assess the impact of odevixibat on serum bile acid levels in patients with ALGS, and evaluate the safety and tolerability of odevixibat in patients with ALGS.

The study consisted of a screening phase and parallel-design treatment period. Two screening visits occurred: the first took place during days −56 to −21 prior to the first dose of study drug and the second occurred during days −49 to ˜14. On day 0, all eligible patients were randomized 2:1 to oral, 120 μg/kg/day odevixibat or once-daily placebo. Eligibility for randomization was determined using pruritus or scratching reports from observer reported outcome (ObsRO)/patient reported outcome (PRO) data for evaluation of itching (PRO), scratching (ObsRO), and sleep disturbance (PRO and ObsRO) in the 14 days before Study Day 1, clinical genetic confirmation of diagnosis, and the liver biochemistry evaluations from the previous screening visits. After written informed consent was obtained, an Interactive Web Response System (IWRS) was used to assign patients to treatment. The randomization codes were computer generated by Albireo or a qualified randomization vendor.

Odevixibat and placebo were supplied as capsules for oral administration. White opaque capsules filled with pellets containing odevixibat or placebo were provided. Two different capsule sizes were available: capsule size 0 that can be opened and capsule size 3 to be swallowed intact. The size 3 capsules were opened only under exceptional circumstances (e.g., patient could not swallow capsule intact). To ensure blinding of treatment assignment, study drug and matching placebo had the same shape and size, with labels on the study drug containers that did not identify the randomized treatment assignment. Dispensing of study drug was coordinated by IWRS.

Treatment was dispensed during on-site clinic visits, and patients or caregivers were instructed to take or administer the study drug at home each morning as an intact capsule(s) (swallowed with a glass of water and with food) or the capsule can be opened and the contents sprinkled and mixed in a small amount of soft room temperature food (e.g., applesauce, followed by water). The double-blind ASSERT treatment period lasted 24 weeks.

Patients who completed the treatment period either attended a follow-up visit 28 days (±7) after the last dose of study drug, or they could continue into an optional open-label extension study (ASSERT-EXT; ClinicalTrials.gov identifier: NCT05035030), in which all patients received odevixibat. There were up to 10 planned clinic visits, including visits for screening, treatment, and follow-up, with one telephone call at week 2 between the randomization and week 4 visits.

### Patients—Key Eligibility Criteria

Patients (of any age) with a genetically confirmed diagnosis of ALGS were eligible for inclusion. Patients must have had a history of significant pruritus, an elevated baseline serum bile acid level, an average caregiver-reported observed scratching or a patient-reported pruritus score ≥2 (on 0 to 4 scale), as measured by the ObsRO instrument (for patients <18 years of age) or the PRO instrument (for patients ≥18 years of age) in the 14 days prior to randomization. Additionally, caregivers or age-appropriate patients (≥8 years of age) agreed to use the electronic diary (eDiary) device to record symptoms.

Exclusion criteria included the following: medical history or ongoing presence of other types of liver disease (e.g., biliary atresia of any kind, progressive familial intrahepatic cholestasis (PFIC), benign recurrent intrahepatic cholestasis, liver cancer or metastasis to the liver on imaging studies); diseases or conditions known to interfere with the absorption, distribution, metabolism (specifically bile acid metabolism), or excretion of drugs in the intestine (e.g., inflammatory bowel disease); chronic (i.e. >3 months) diarrhea; active, clinically significant, acute or chronic infection or infection requiring hospitalization or parenteral anti-infective treatment within 4 weeks of treatment start; cancer within the last 5 years except for basal cell carcinoma; cancer >5 years prior to screening except for non-liver cancers with no evidence of recurrence; or chronic kidney disease. Patients were excluded from the study if they had biliary diversion surgery within 6 months prior to start of screening period; had a liver transplant or one planned within 6 months of randomization; signs of decompensated liver disease (e.g., ascites); or pruritus caused by any condition other than ALGS (e.g., refractory atopic dermatitis, other primary pruritic skin disease). Use of resins or medications that slow gastrointestinal motility were not permitted. Patients with laboratory parameters above the following thresholds were excluded: international normalized ratio (INR)>1.4, serum alanine aminotransferase (ALT)>10 times the upper limit of normal (ULN) at screening, serum ALT >15 times the ULN during the last 6 months, and total bilirubin >15 times the ULN at screening. Additional exclusion criteria included any patient who was pregnant or lactating or who was planning to become pregnant within 24 weeks of randomization; patients with a past medical history of alcohol or substance abuse; and patients that have had investigational exposure to a drug, biologic agent, or medical device within 30 days prior to screening, or 5 half-lives of the study agent, whichever was longer.

### Assessments

A single primary endpoint was evaluated. The primary endpoint in this study was the change from baseline in scratching to Month 6 (Weeks 21 to 24) as measured by the observer-reported outcome (ObsRO) instrument.

The study was also powered for a second primary endpoint, the change in serum bile acid levels from baseline to the average of week 20 and week 24. Additional secondary efficacy endpoints were as follows:
Change from baseline in pruritus to Month 6 (Weeks 21 to 24) as measured by the Albireo PRO instrument.
Percentage of patients achieving a clinically meaningful decrease in pruritus (pruritis responders) as measured by the ObsRO/PRO instruments.
Change from baseline through Week 24 in patient reported and observer reported itching and scratching severity scores, respectively, for the morning assessment and for the evening assessment; sleep parameters as measured with the Albireo ObsRO/PRO instruments (e.g., tiredness and number of awakenings); and PedsQL subdomain scores.
Assessment of Global Symptom Relief to from baseline to Weeks 4, 12, and 24 as measured by patient, caregiver, and clinician Global Impression of Symptoms GIS (PGIS, CaGIS, CGIS) items.
Change from baseline to Week 24 in xanthomatosis as assessed by the Clinician Xanthoma Scale; serum bile acid levels; serum ALT concentration; and serum AST concentration; gamma-glutamyl transferase concentration; total bilirubin concentration.
Change from baseline in biochemical markers and measures of bile acid synthesis (autotaxin, p-C4, only in patients >10 kg); and total cholesterol concentration.

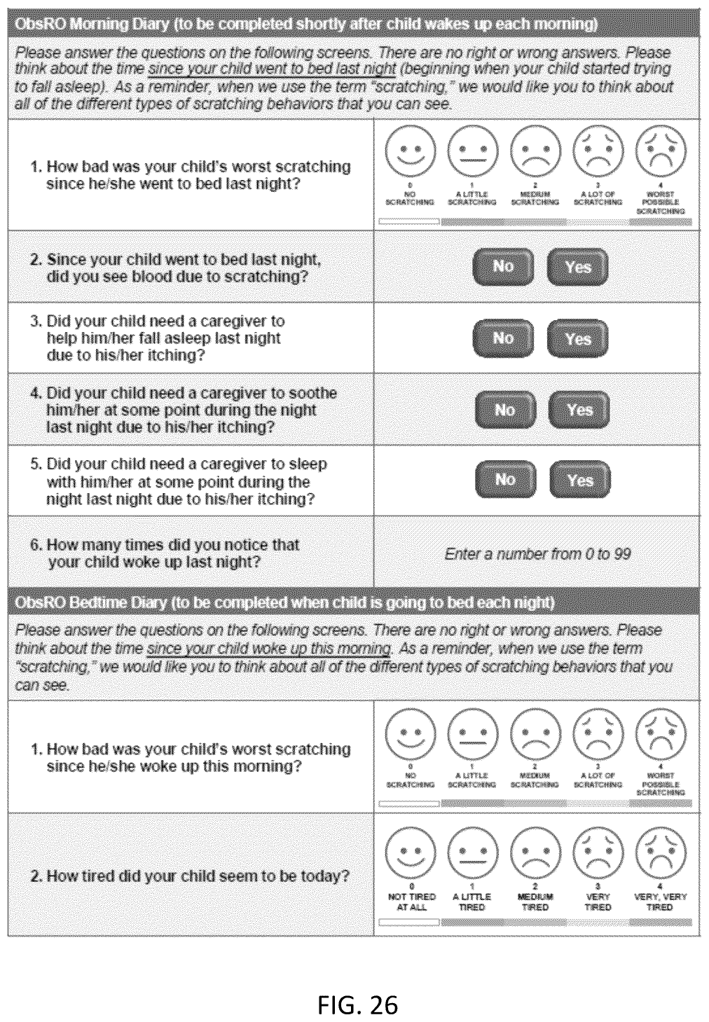

Itching, Scratching, and Sleep Parameters. Itching, observed scratching, and sleep disturbance were recorded twice each day via eDiary. Patients and/or caregivers were instructed to complete the eDiary every day in the morning after the patient woke and in the evening just before the patient went to sleep throughout the study. The eDiary included ObsRO and PRO items. The PRO items assessed severity of itch, aspects of sleep disturbance (morning diary only), and tiredness. The ObsRO items assessed severity of observed scratching, aspects of observed sleep disturbance (morning diary only), and signs of tiredness (evening diary only). See FIG. 26. The ObsRO and PRO scratching and itch severity items used 0 to 4 response scales.

Serum Bile Acids. Blood samples for analysis of fasting total serum bile acid were drawn at all clinic visits. All post-baseline serum bile acid was blinded. Samples were processed and transported to a central laboratory per instructions in the laboratory manual.

Biochemical Markers and PK Samples. Blood samples for analysis of clinical chemistry were drawn during Screening Visits 1 and 2, as well as at randomization (Study Day 1), Week 4 to 24/EOT, and Safety Follow-up visits. As outlined in Table 1, total bilirubin, AST, ALT, and gamma glutamyl transferase assessments were included as part of the routine laboratory parameters. Blood samples for autotaxin and p-C4 were drawn at randomization (Study Day 1), Week 12, and Week 24/EOT, only for children with body weight >10 kg. Blood for odevixibat PK assessment was drawn at Week 4, Week 20, and EOT (only for children with body weight >10 kg). Samples were processed and transported to a laboratory per instructions in the laboratory manual.

TABLE 1
Routine Laboratory Parameters.

| Clinical Chemistry | Hematology | Urinalysis |
|---|---|---|
| Albumin | Hematocrit | Blood |
| ALT | Hemoglobin | Glucose |
| Alkaline phosphatase (and | Platelet count | Ketones |
| isoenzymes as applicable) | Red blood cell count | Leukocytes |
| AST | White blood cell count | Nitrites |
| Bilirubin - total and direct | and differential | pH |
| Blood Urea Nitrogen | (neutrophils, | Protein |
| Calcium | eosinophils, basophils, | |
| Chloride | lymphocytes, and | |
| Cholesterol | monocytes) | |
| Creatinine | | |
| Creatine kinase | | |
| Gamma-glutamyl transferase | | |
| Potassium | | |
| Sodium | | |
| Triglycerides | | |

ALT = alanine aminotransferase; AST = aspartate aminotransferase.

Global Impression of Change and Global Impression of Symptom Measures. Patients (≥8 years of age), caregivers, and clinicians completed global impression of change (GIC) measures (patient global impression of change (PGIC), caregiver global impression of change (CaGIC), and clinical global impression of change (CGIC)) and the global impression of symptoms (GIS) measures (patient global impression of symptoms (PGIS), caregiver global impression of symptoms (CaGIS), clinical global impression of symptoms (CGIS)) at randomization (Study Day 1; PGIS only), Week 4, 12, and 24/EOT.

The GIC items assessed changes in itch (patient version), scratching (caregiver and clinician versions), and sleep (all versions) since starting the study drug. The GIS items assessed itch (patient version), scratching (caregiver and clinician versions), and sleep (all versions) in the past week. Caregivers and clinicians completed the GIC and GIS for all patients; those patients ≥8 years of age completed the patient version.

Clinical Assessment of Xanthoma. Changes in xanthoma as assessed by the Clinician Xanthoma Scale were measured at randomization (Study Day 1), Week 12, and Week 24/EOT visits. The clinician's assessment of the participant's xanthomatosis was focused on the number of lesions present and the degree to which the participant's lesions interfered or limited activities. The clinician xanthoma scale used a 5-point scale, in which 0 represented no evidence of xanthomatosis, 1 represented fewer than 20 scattered individual lesions, 2 represented more than 20 lesions that did not interfere with or limit activities, 3 represented large numbers of lesions that by their large numbers or size caused distortion of the face or extremities, and 4 represented xanthomas that interfered with function (such as hand use or ability to walk) because of excess size or number.

Safety. The primary safety analysis for this study included the incidence of total treatment-emergent adverse events (AEs) and treatment-emergent adverse events (TEAEs) categorized by causality, severity, and seriousness assessments made by the investigator by comparing study drug exposure to placebo. Other safety assessments included physical examinations, concomitant medications, vital signs, laboratory tests, and liver ultrasound and elastography.

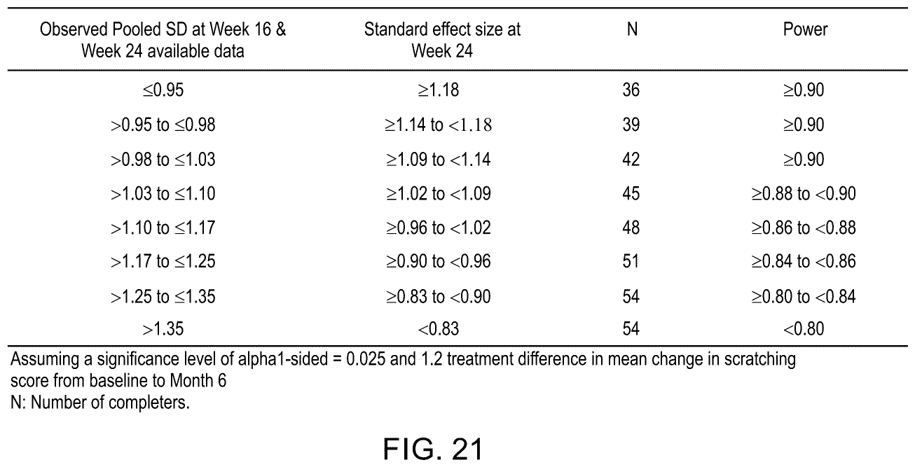

Statistical Analyses. The planned sample size of forty-five (45) patients <18 years of age were randomized at an experimental to control allocation of 2 to 1 in order to obtain approximately 36 completers, assuming an approximate drop-out rate of 20%. Subjects <18 years of age were randomized according one age stratification factor, i.e., <10, and 10 to <18 years of age. This stratification factor was based on showing an increase in the prevalence and severity of pruritus in children <10 years of age. At a 1-sided significance level of alpha 1-sided=0.025, assuming a pooled standard deviation (SD) of 1.0, and a difference between the treatment groups of 1.2 in change of pruritus, favoring response, the power of the study was 0.909, using the exact method (Proc Power, SAS v.9.4, Cary, NC). The key secondary endpoint was also powered for a standardized treatment effect (treatment effect/SD) of 1.2. After a minimum of 18 patients had completed the Week 16 visit, the pooled SD of change from baseline to Month 4 (Weeks 13-16 assessments) and available change from baseline to Month 6 (Weeks 21-24 assessments) in average monthly scratching score was calculated by an independent statistician to assess the need for sample size re-estimation. Sample size re-estimation was based on noncomparative blinded data for which no alpha adjustment was required. If the pooled SD was underestimated in this sample size calculation, an adjustment would be made to maintain study power, assuming a 1.2 treatment effect. It was assumed that the Week 16 and Week 24 SDs would be equivalent; however, a multiplication factor (1.07) on the calculated SD may be used based on the current understanding of the instrument at the time of sample size re-estimation. If SD was overestimated in this original sample size calculation, the sample size would not be decreased. The planned sample size re-estimation was conducted based on FIG. 21; the sample size was increased to target 48 completers (i.e. approximate 32 and 16 in the odevixibat and placebo groups, respectively, based on 2:1 randomization). Given the low actual dropout rate at that time, 7 patients were added to the study. A total of 52 patients were enrolled.

### Results

### Patients

Patient Disposition. Study disposition and baseline characteristics are provided in FIGS. 1-3. A total of 52 patients were randomized; 17 and 35 to placebo and odevixibat 120 μg/kg/day, respectively. Overall, all 52 (100%) of patients completed the 24-week treatment period (FIG. 2).

Patient demographics and baseline characteristics are summarized in FIG. 3. The mean age of the patients was 6.3 years; 27 (52%) were male. 92% of patients had a mutation in JAG1 and 8% of patients had a mutation in NOTCH2. At baseline (study entry), 89% of patients were treated with ursodeoxycholic acid (UDCA) and 98% of patients were treated with an anti-pruritus medication. Baseline mean (SD) pruritus score (range: 0-4) was 2.9 (0.6). Baseline mean (SD) bile acid levels were 240 (116) μmol/L. Baseline mean (SD) ALT, AST and bilirubin levels were 174 (84) U/L, 167 (83) U/L, and 3.2 (2.8) mg/dL, respectively. See FIG. 3.

### Efficacy

### Primary and Secondary Endpoints

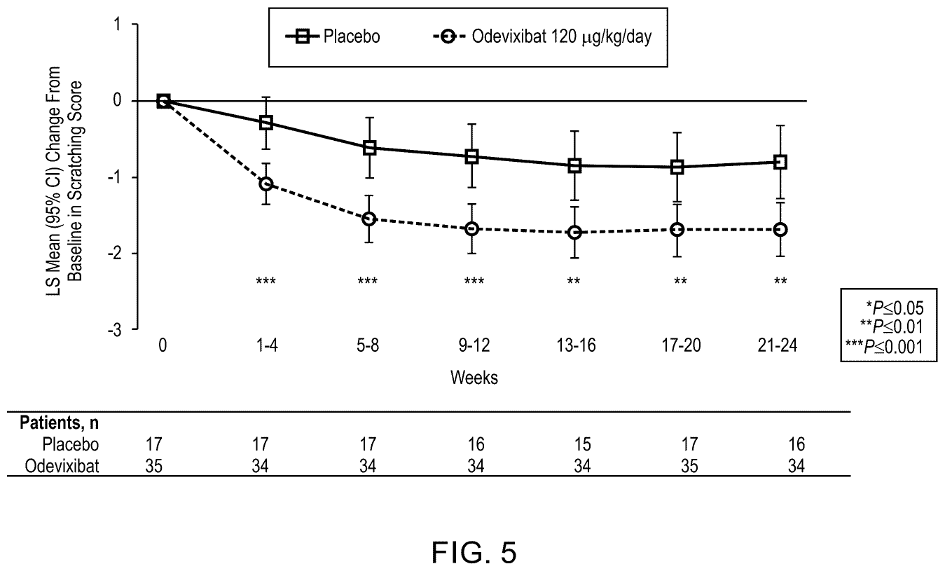

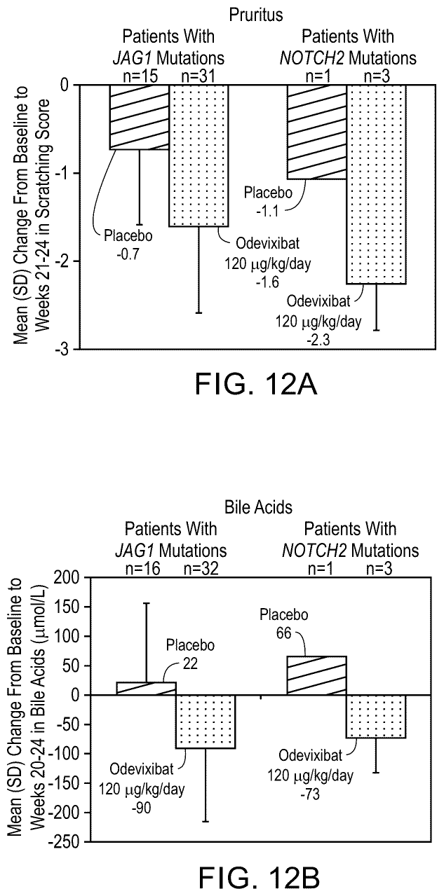

The study met the primary endpoint. The primary endpoint of change from baseline in scratching score to Month 6 (Weeks 21-24) as measured by the Albireo ObsRO caregiver instrument. Significant improvements in pruritus were observed with odevixibat versus placebo (FIGS. 4 and 5). Treatment with odevixibat overall, at 120 μg/kg/day, led to statistically significant improvements in pruritus compared with placebo from baseline to the weeks 21-24 of treatment period based on the ObsRO instrument: the least square (LS) mean change from baseline to Weeks 21-24 in scratching score was −1.69 points (P=0.0024; 34 patients in the odevixibat 120 μg/kg/day group) compared to 16 patients who were treated placebo (−0.8 point reduction at Weeks 21-24) (FIGS. 4 and 5). Improvements in pruritus were achieved at Weeks 21-24 in patients with JAG1 and NOTCH2 mutations (FIG. 12A) who received odevixibat versus placebo.

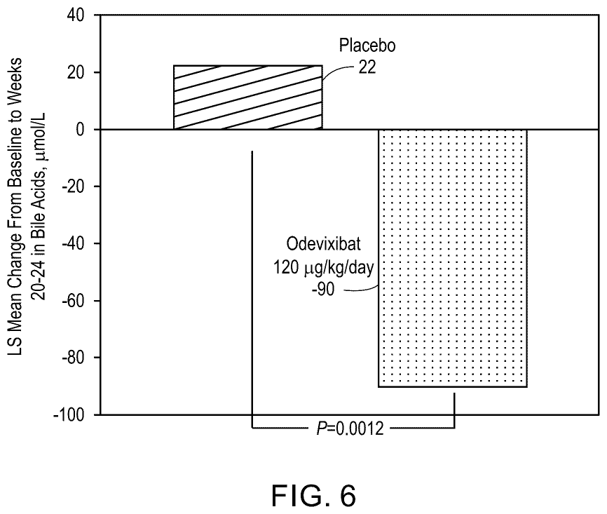

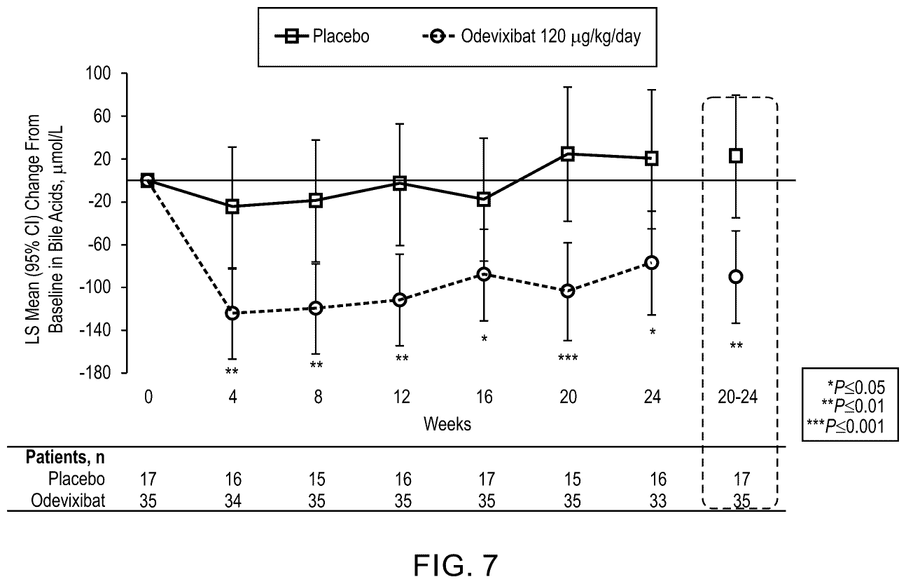

Serum bile acid reduction was also significantly larger in the odevixibat group compared with placebo to the average of Weeks 20 and 24 (P=0.0012; FIG. 6). The mean reduction in serum bile acid levels was 90 μmon in patients treated with odevixibat vs. an increase of 22 μmon in those treated with placebo (FIGS. 6 and 7). Serum bile acid levels were also improved at Weeks 20-24 in patients with JAG1 and NOTCH2 mutations (FIG. 12B) who received odevixibat versus placebo.

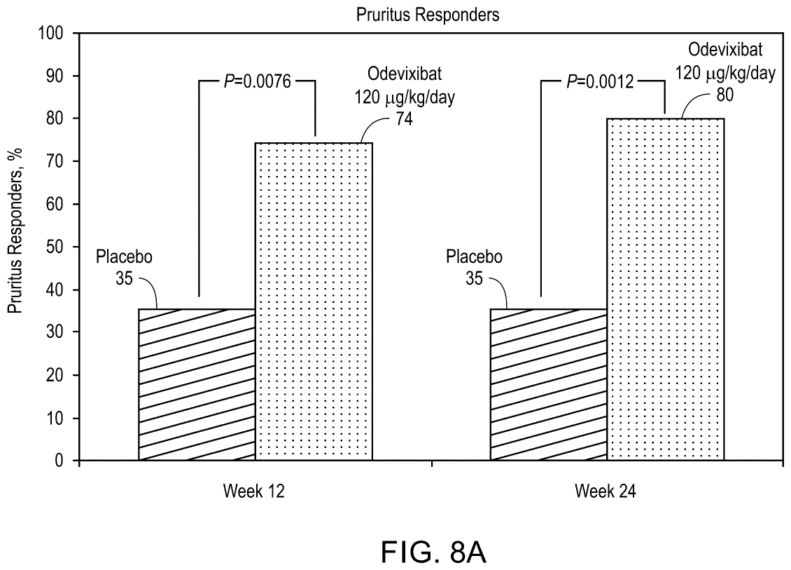

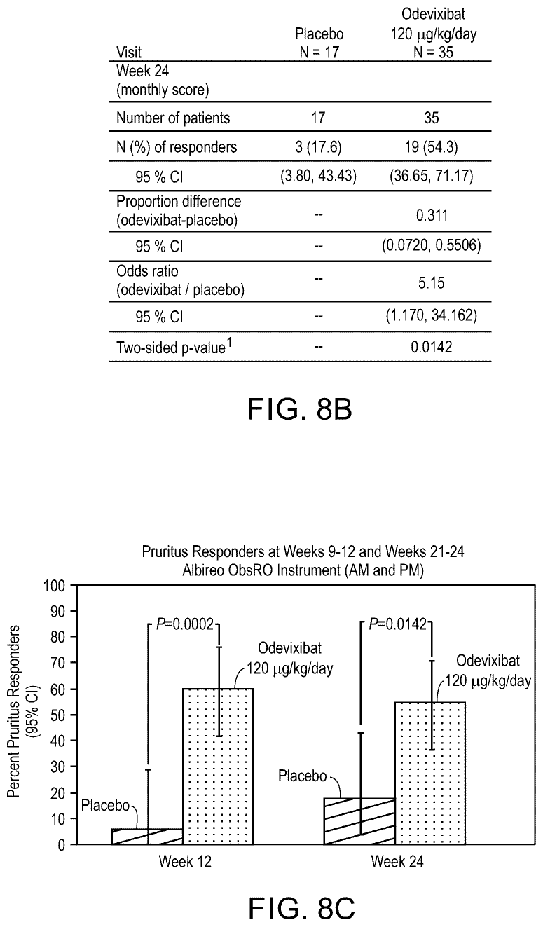

Improvement in pruritus among odevixibat-treated patients based on mean monthly ObsRO scratching score was observed by as early as week 1 of treatment; the percentage of patients who were pruritus responders (i.e. that had a greater than or equal to 1-point reduction in monthly scratching score from baseline) at week 12 was 74% with odevixibat vs 35% with placebo (p=0.0076), and at week 24 was 80% with odevixibat vs 35% with placebo (p=0.0012) (FIGS. 8A-8C).

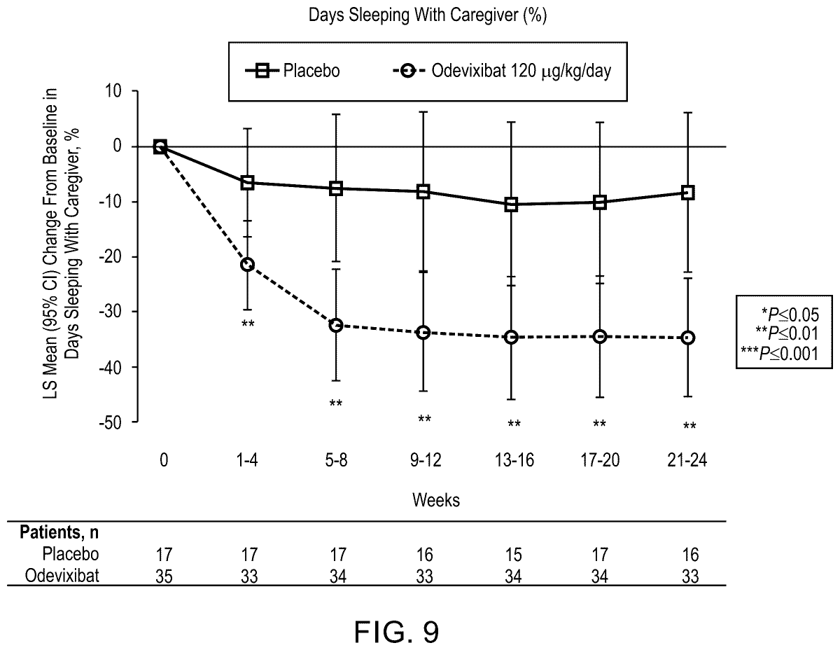

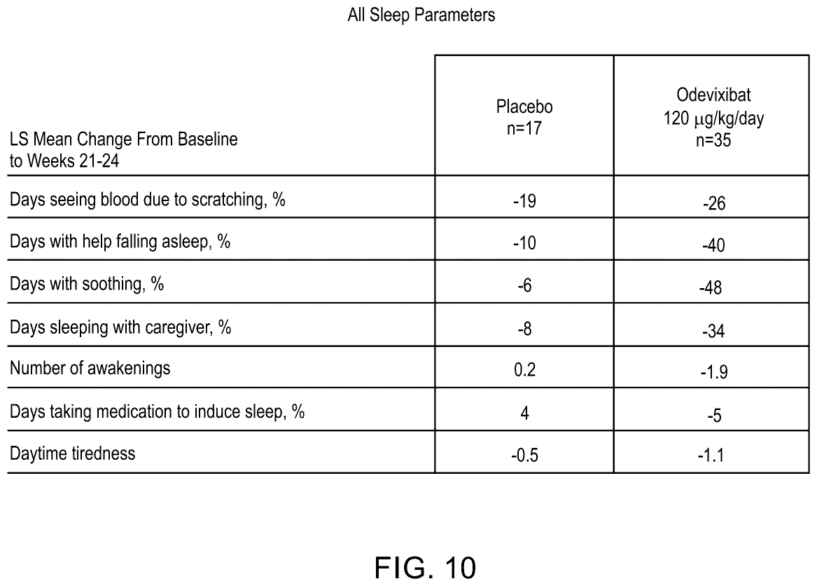

Consistent with improvements observed with pruritus, treatment with odevixibat improved sleep parameters for patients based on caregiver-reported information. At weeks 21-24, the LS mean change from baseline for percent of days with help falling asleep, soothing and sleeping with caregiver were −43%, −47% and −35%, respectively, for the odevixibat group compared to −10%, −6% and −8%, respectively for the placebo group (one-sided p≤0.0017) (FIGS. 9 and 10).

Additionally, caregivers rated patients' daytime tiredness using a 5-point scale that ranged from 0 (“not tired at all”) to 4 (“very, very tired”). A greater mean reduction (i.e., improvement) from baseline to weeks 21 to 24 was observed with odevixibat compared with placebo (−1.1 versus −0.5, respectively; one side p value=0.0062). (FIG. 10)

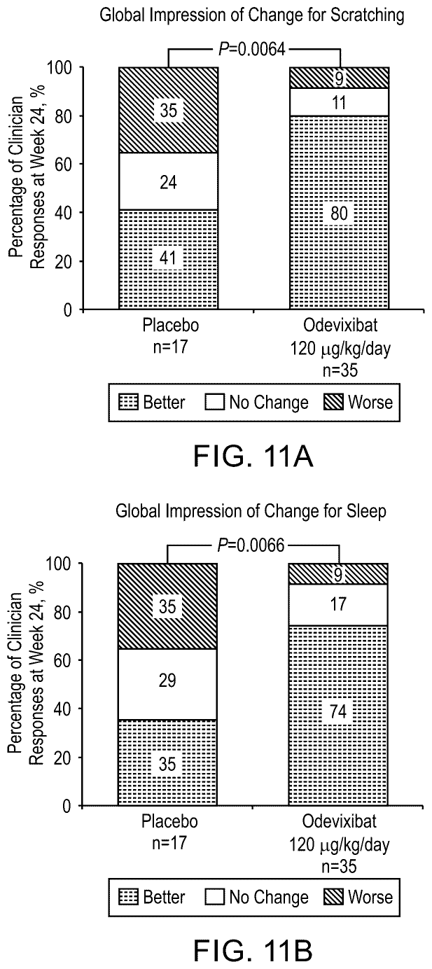

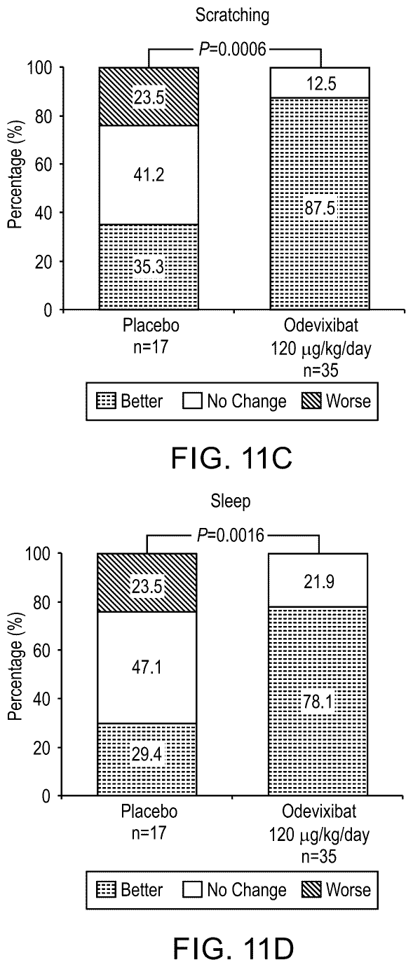

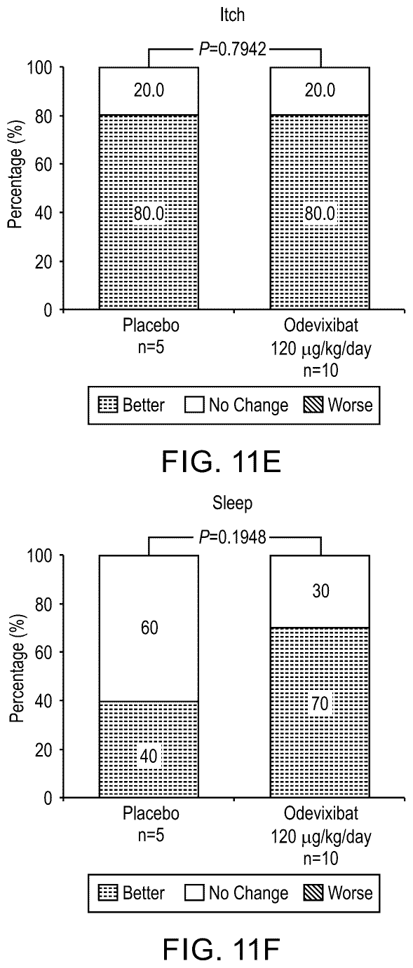

Consistent with improvements observed with pruritus and sleep parameters for patients based on clinician-reported information, treatment with odevixibat versus placebo improved CGIC for scratching and CGIC for sleep (FIGS. 11A and 11B) in patients (percentage of clinician responses at week 24 of the CGIC for scratching was 80% for odevixibat versus 41% for placebo, and the percentage of clinician responses at week 24 of the CGIC for sleep was 74% for odevixibat versus 35% for placebo). Consistent with improvements observed with pruritus and sleep parameters for patients based on clinician-reported information, treatment with odevixibat versus placebo improved CaGIC for scratching and CaGIC for sleep (FIGS. 11C and 11D) in patients (percentage of caregiver responses at week 24 of the CaGIC for scratching was 87% for odevixibat versus 35% for placebo, and the percentage of caregiver responses at week 24 of the CaGIC for sleep was 78% for odevixibat versus 29% for placebo). Consistent with improvements observed with pruritus and sleep parameters for patients based on patient-reported information, treatment with odevixibat versus placebo improved PGIC for itching and PGIC for sleep (FIGS. 11E and 11F) in patients (percentage of patient responses at week 24 of the PGIC for itching was 80% for odevixibat and placebo, and the percentage of patient responses at week 24 of the PGIC for sleep was 70% for odevixibat versus 40% for placebo).

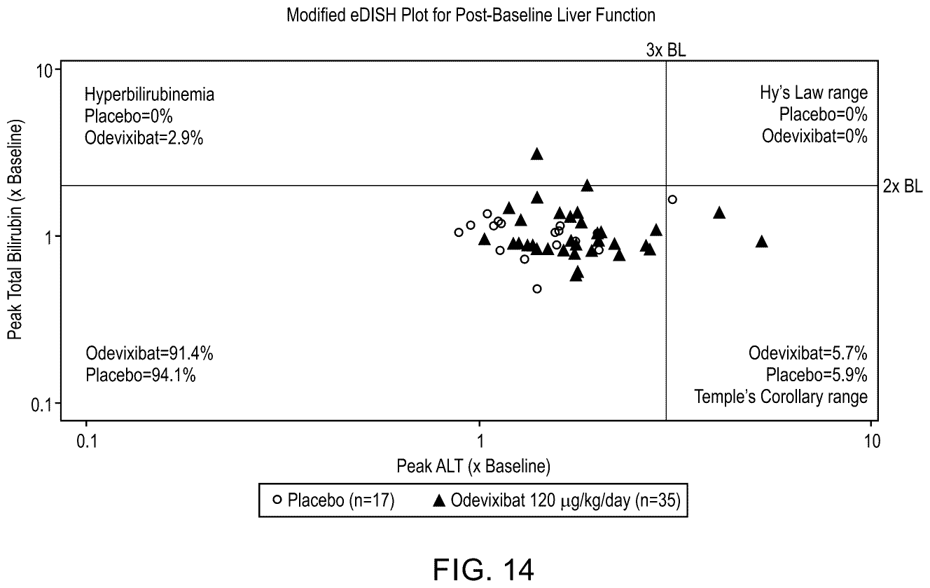

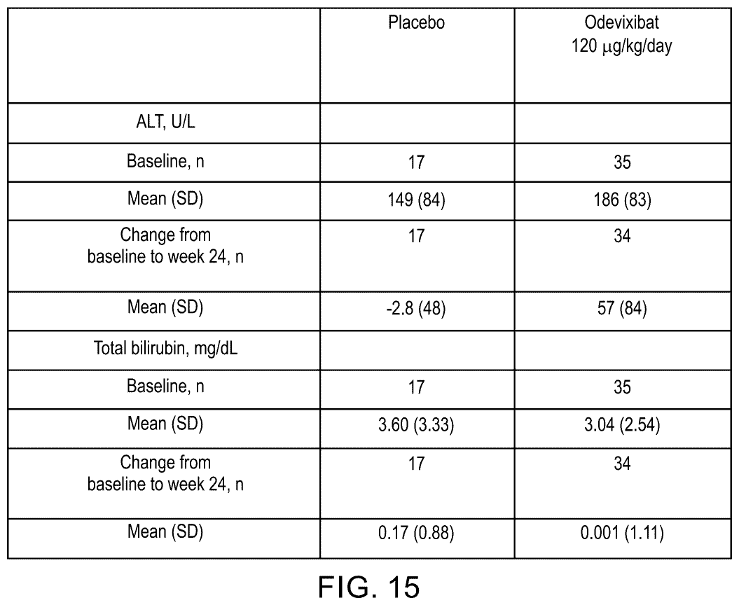

Post-Baseline liver function in those treated with odevixibat or placebo are presented in FIG. 14. No patient had post-treatment changes in Total Bilirubin (≥3×BL) concurrently with ALT (≥2×BL) (no Hy's Law or drug induced liver injury cases). Mean changes from baseline by group to week 24 in hepatic biochemical parameters are presented in FIG. 15. See Table 2 for a summary of the changes from baseline to week 24 in hepatic parameters, biochemical markers, and markers of bile acid synthesis.

TABLE 2
Changes from baseline to week 24 in hepatic parameters,
biochemical markers, and markers of bile acid synthesis.

| | Placebo | Odevixibat |
|---|---|---|
| | n = 17 | n = 35 |
| Total bilirubin, μmol/L | | |
| Mean (SD) at baseline | 62 (57) | 52 (43) |
| Mean (SD) at week 24 | 64 (60) | 53 (42) |
| LS mean (SE) change from | 4 (4) | 1 (3) |
| baseline to week 24 | | |
| Serum AST, U/L | | |
| Mean (SD) at baseline | 161 (91) | 170 (81) |
| Mean (SD) at week 24 | 151 (66) | 217 (131) |
| LS mean (SE) change from | −2 (20) | 54 (15) |
| baseline to week 24 | | |
| Serum ALT, U/L | | |
| Mean (SD) at baseline | 149 (84) | 186 (83) |
| Mean (SD) at week 24 | 146 (66) | 245 (121) |
| LS mean (SE) change from | 4 (19) | 66 (14) |
| baseline to week 24 | | |
| GGT, U/L | | |
| Mean (SD) at baseline | 536 (345) | 366 (211) |
| Mean (SD) at week 24 | 594 (461) | 460 (270) |
| LS mean (SE) change from | 62 (61) | 100 (44) |
| baseline to week 24 | | |
| Autotaxin, ng/mL | | |
| Mean (SD) at baseline | 2070 (1023) | 1846 (540) |
| Mean (SD) at week 24 | 1905 (719) | 1452 (440) |
| Mean (SD) change from | −45 (687) | −414 (439) |
| baseline to week 24 | | |
| C4, ng/mL | | |
| Mean (SD) at baseline | 7 (6) | 6 (6) |
| Mean (SD) at week 24 | 8 (9) | 19 (13) |
| Mean (SD) change from | 1 (10) | 12 (12) |
| baseline to week 24 | | |
| Total cholesterol, mmol/L | | |
| Mean (SD) at baseline | 9 (5) | 8 (2) |
| Mean (SD) at week 24 | 10 (8) | 7 (2) |
| LS mean (SE) change from | 1 (1) | −1 (1) |
| baseline to week 24 | | |

None of the 52 patients underwent surgical interruption of the enterohepatic circulation or liver transplantation during the ASSERT placebo-controlled study.

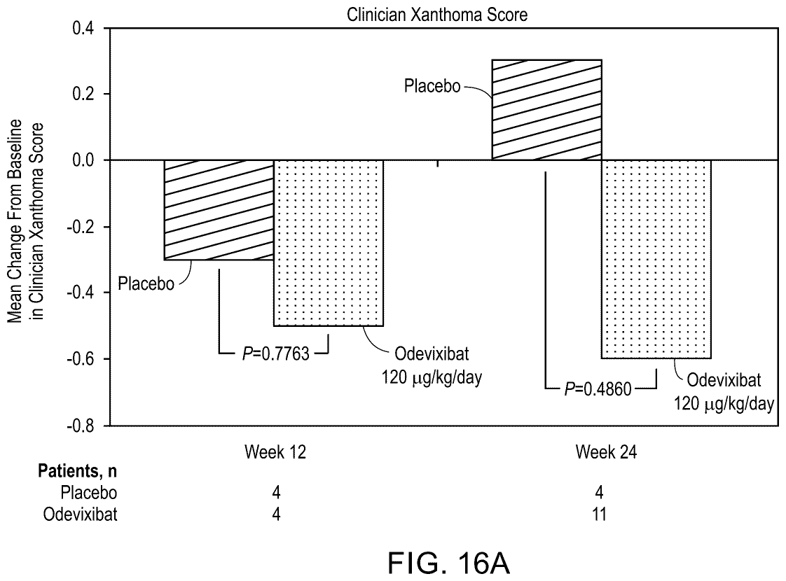

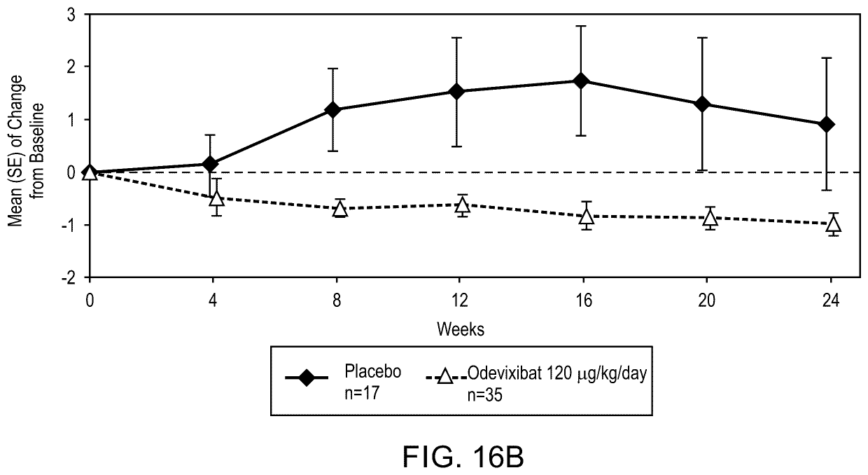

Improvement in clinician xanthoma score among odevixibat-treated patients who had xanthomas (n=11) was observed. The mean change from baseline to week 12 and end of treatment in the clinician xanthoma score is shown in FIG. 16A. Mean (SE) improvements from baseline to week 24 for odevixibat vs placebo is demonstrated in FIG. 16B. In the odevixibat group, 27/35 (77%) patients had no change from baseline to week 24 in xanthomas, including 25 patients who had no xanthomas at any time point during the study. An additional seven (20%) patients showed improvements in xanthomas with odevixibat, and one (3%) patient's score increased from 0 to 1 on the Clinician Xanthoma Scale. In the placebo group, 14/17 (82%) patients had no change from baseline to week 24 in xanthomas, with all 14 having no xanthomas at any time point. Of the remaining three patients in the placebo group, two (12%) showed improvements in xanthomas (score decreased from 2 to 1 in one patient and 3 to 1 in another patient) and one (6%) showed worsening (score increased from 0 to 3).

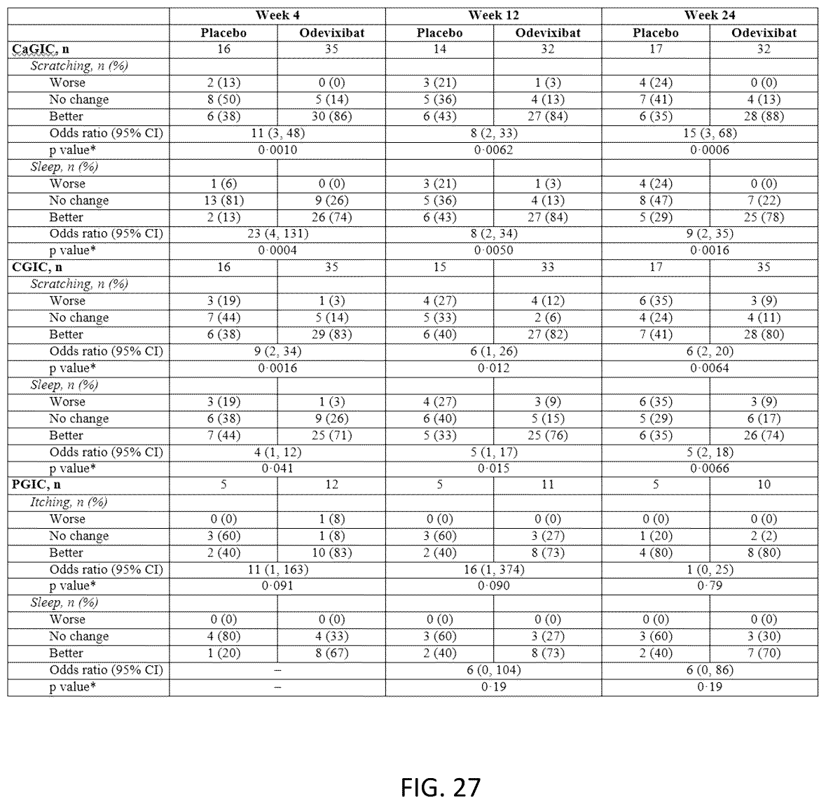

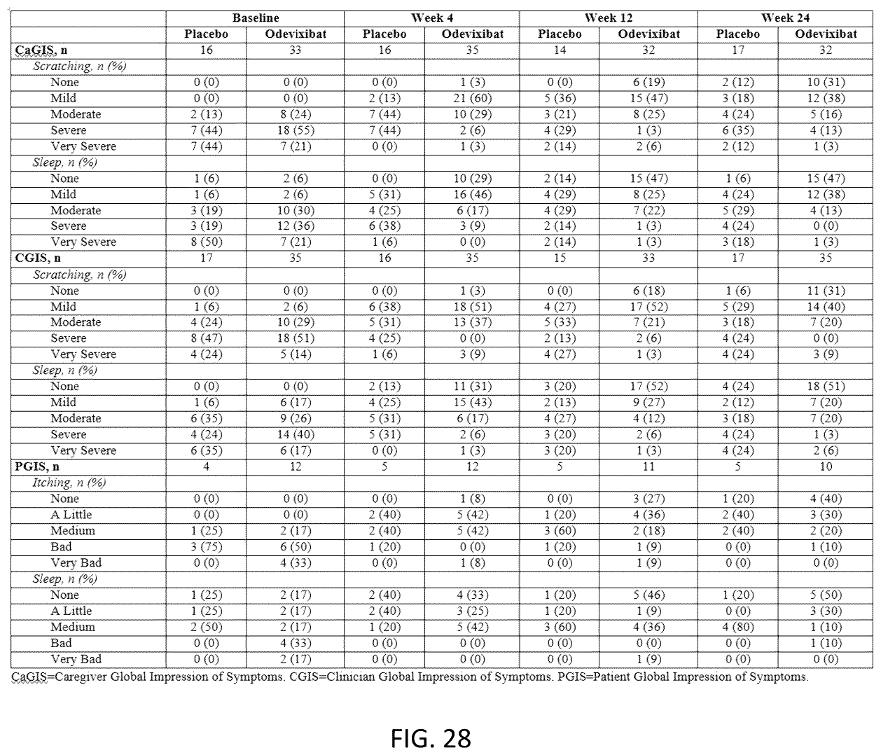

Instruments assessing quality of life (i.e., PedsQL) and global symptom relief (i.e., GIC and GIS) also supported the efficacy of odevixibat. See Table 3 and FIGS. 27 and 28. For example, caregivers completing the CaGIC indicated that a higher proportion of odevixibat-treated patients had improved pruritus and sleep at week 24 relative to patients receiving placebo (pruritus: 28/32 [88%] vs 6/17 [35%] with odevixibat vs placebo; sleep: 25/32 [78%] vs 5/17 [29%] with odevixibat vs placebo). Based on these data, the odds of improvement versus no change or worsening in scratching at week 24 were 15 times higher for odevixibat-treated patients relative to patients receiving placebo (p=0.0006); similarly, the odds of improvement in sleep at week 24 were nine times higher for odevixibat-treated patients relative to patients receiving placebo (p=0.0016). Finally, according to a post hoc analysis of the exit survey administered at the completion of the study, a higher proportion of caregivers of patients in the odevixibat group reported meaningful change in the patient since the start of treatment (25/32 [78%] vs 4/15 [27%] for odevixibat vs placebo, respectively, p=0.0009).

TABLE 3
Changes from baseline in PedsQL total and domain scores.

| | Placebo | Odevixibat | p |
|---|---|---|---|
| PedsQL | n = 11 | n = 30* | value^{†} |
| Total Score | | | |
| Mean (SD) at baseline | 67.1 (13.5) | 67.8 (16.3) | — |
| LS mean (SE) change from | 10.7 (4.0) | 13.5 (2.5) | 0.54 |
| baseline to week 24 | | | |
| Physical Functioning | | | |
| Mean (SD) at baseline | 73.6(16.1) | 71.6 (21.4) | — |
| LS mean (SE) change from | 9.8 (4.4) | 14.2 (2.9) | 0.38 |
| baseline to week 24 | | | |
| Emotional Functioning | | | |
| Mean (SD) at baseline | 55.0 (22.0) | 55.8 (17.8) | — |
| LS mean (SE) change from | 15.7 (5.1) | 21.0 (3.3) | 0.37 |
| baseline to week 24 | | | |
| Social Functioning | | | |
| Mean (SD) at baseline | 77.3 (14.6) | 78.5 (18.8) | — |
| LS mean (SE) change from | 10 (5.0) | 5.1 (3.2) | 0.38 |
| baseline to week 24 | | | |
| School Functioning | | | |
| Mean (SD) at baseline | 58.2 (18.8) | 63.3 (22.9) | — |
| LS mean (SE) change from | 9.8 (4.8) | 13.3 (3.0) | 0.52 |
| baseline to week 24 | | | |

Scores range from 0 and 100, with higher scores indicating better functioning. *n=28 for LS mean change from baseline to week 24. ^{†}Analysis of change from baseline with odevixibat vs placebo based on ANCOVA model at each visit with baseline score as a covariate and treatment group and age category (<5 years, 5-7 years, 8-12 years, 13-18 years) as fixed effects. ANCOVA=analysis of covariance. LS=least squares. PedsQL=Paediatric Quality of Life Inventory.

### Safety

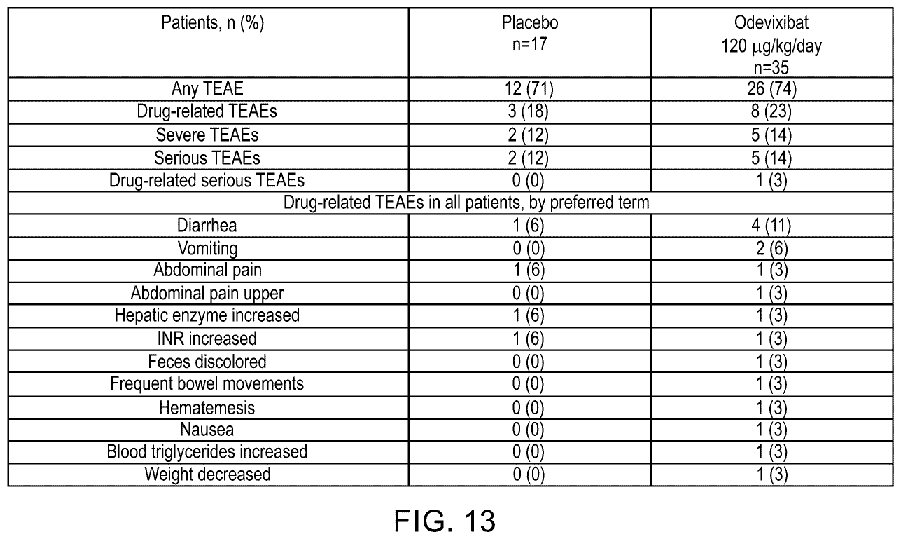

Odevixibat at a dose of 120 μg/kg/day was well tolerated over 24 weeks in patients with ALGS. Overall, 26 (74%) of the 35 patients receiving odevixibat experienced at least one treatment ending adverse event (TEAE); a similar rate was observed in patients receiving placebo (12/17 (71%); FIG. 13). Most TEAEs were mild or moderate in severity and assessed as unrelated to study drug. Incidence of drug-related TEAEs was 23% for odevixibat-treated patients and 18% for placebo-treated patients. Incidence of treatment-emergent diarrhea was 29%, in the odevixibat group, and 6% in the placebo group. The most commonly reported drug-related TEAE (occurring in ≥10% of patients overall) was diarrhea (odevixibat vs. placebo: 11% vs 6%). The incidence of SAEs was generally similar in the odevixibat (14.3%) and placebo (11.8%) groups. No deaths or treatment-emergent adverse events leading to study drug discontinuation were reported. No patients discontinued the placebo-controlled study.

Infections were the most common types of TEAEs reported across the treatment groups. The incidence of infections was similar in patients who received odevixibat (51.4%) compared to patients who received placebo (58.8%). Commonly reported infections in patients in the odevixibat group were generally typical of those in a paediatric population and included COVID-19 (14.3%) and upper respiratory tract infection, respiratory tract infection, and bronchitis (8.6% each). Most infections were assessed as Grade 1 to 2 in intensity. Serious infections were reported in 3 patients in the odevixibat group, including rhinovirus infection, tonsillitis, pneumonia, and otitis media, and 1 patient in the placebo group who developed a subcutaneous abscess and chronic otitis media.

Gastrointestinal disorders were also commonly reported. The incidence of GI disorders was higher in the odevixibat group (34%) compared to the placebo (12%) group. The most common GI disorders among patients who received odevixibat were diarrhoea (29%) and abdominal pain (preferred terms of abdominal pain and abdominal pain upper) (14%). All cases of diarrhoea and most reports of abdominal pain were Grade 1 in intensity; none of these events led to treatment interruption, dose reduction, or discontinuation from study treatment.

Patients with ALGS have ongoing liver damage due to cholestasis with elevated serum bile acids, liver transaminases, and bilirubin levels. There were no cases of liver decompensation events during the study and the incidence of liver-related events, which included a comprehensive evaluation of events across SMQs, was similar in the odevixibat (11%) and placebo (12%) groups. Review of changes from baseline to Week 24 for transaminase levels showed larger mean increases for ALT, AST, and GGT for patients who received odevixibat compared to patients who received placebo. The increases were observed by Week 4 of treatment and then plateaued through Week 24. Changes from baseline to Week 24 in total bilirubin were minimal and similar in the odevixibat and placebo groups. Elevations in transaminase levels have been reported in patients with ALGS following partial surgical biliary diversion, patients receiving cholestyramine, and patients receiving another IBAT inhibitor, maralixibat.

Based on this finding and that almost all patients had elevated levels of transaminases and total bilirubin at study entry, further evaluations were conducted, including eDISH plots. Based on these further evaluations, 3 patients, including 2 in the odevixibat group and 1 in the placebo group had ALT and AST elevations ≥3× baseline without concurrent (within 30 days) elevations in total bilirubin ≥2× baseline. A review of the pertinent clinical and diagnostic information for the 2 patients treated with odevixibat suggests that the occurrence of DILI in these patients is unlikely. One patient interrupted study drug for the hepatic enzyme elevations with return to baseline levels off treatment and was rechallenged with fluctuating transaminase levels after restarting treatment, but all lower than the peak observed, without further interruption in study treatment. The other patient continued dosing with odevixibat with improvement of the ALT and AST elevations on-treatment without intervention.

Patients with ALGS are known to have fat-soluble vitamin deficiencies due to decreased delivery of bile acids into the intestine and malabsorption. No clinically meaningful changes from baseline to the last assessment were observed in mean vitamin levels or INR. Two patients, 1 in each treatment group, had a fat-soluble vitamin deficiency TEAE that was reported by the Investigator as refractory to clinically recommended vitamin supplementation. Both had vitamin D deficiency reported and the patients responded to modification in their vitamin supplementation with resolution of the deficiency. Shifts to low vitamin D levels were reported in 8 patients on odevixibat. None of these patients were taking the protocol-recommended supplementation at the time of the shift. Shifts to low vitamin E levels were reported in 4 patients on odevixibat, none of whom were taking the protocol-recommended supplementation at the time of randomisation or at the time of the shifts.

For INR, 12 patients in the odevixibat group shifted to high INR based on the normal range; 6 of these patients had clinically meaningful shifts as defined in the protocol (INR >1.2). Only 1 of these 6 patients was receiving adequate vitamin K supplementation. Review of potential clinical sequelae indicated that 1 of the patients with INR >1.2, who was not receiving vitamin supplementation at study entry, experienced haematemesis that responded rapidly to treatment with phytomenadione.

Possible sequelae of fat-soluble vitamin deficiency were reported with similar incidence in the odevixibat (14%) and placebo (18%) groups. The most common sequelae were haematoma (9% in the odevixibat group), INR increased (3% and 12% in the odevixibat and placebo groups), and epistaxis (12% in the placebo group). Most of the possible sequelae were reported as Grade 1 or 2 in intensity and did not lead to treatment interruption, dose reduction, or discontinuation from study treatment.

Review of hematology, coagulation, clinical chemistry, and urinalysis data did not reveal any clinically meaningful changes from baseline with results similar in the odevixibat- and placebo-treated patients. Further, changes from baseline in vital signs were similar in the odevixibat and the placebo groups.

### Conclusions

The primary and key secondary endpoints (pruritus improvement and bile acid level reduction) were achieved in this phase 3, placebo-controlled trial of odevixibat treatment in patients with ALGS. Treatment with odevixibat for 24 weeks led to highly statistically significant and clinically meaningful improvements in pruritus, as well as significant reductions in bile acid levels and improvements in most sleep parameters relative to placebo. Treatment effects were early, rapid, and sustained. Odevixibat at a dose of 120 μg/kg/day was generally well tolerated over 24 weeks with most TEAEs being mild to moderate in severity and not dose limiting. No patients discontinued the study and 96% of patients rolled over into the open-label extension study.

Odevixibat-associated reductions in pruritus severity were statistically significant and clinically meaningful according to multiple metrics. In addition to the pruritus responder analysis, data from the ObsRO sleep items, caregiver and clinician versions of the GIC, and exit interviews all support the clinical efficacy of odevixibat. As pruritus is a major driver of liver transplantation in patients with ALGS, these results suggest odevixibat may have the potential to delay or prevent liver transplantation. Odevixibat may be a safe, effective, non-surgical option to reduce the systemic accumulation of bile acids that results from cholestasis, lessen the severity of pruritus, and ultimately improve the standard of care in patients with ALGS.

### Example 2. An Open Label Study to Evaluate the Long-term Safety and Efficacy of Odevixibat in Patients with Alagille Syndrome (ASSERT-EXT)

ALGS is characterized by one or more of a number of organ manifestations including:
Hepatic manifestations: cholestasis, bile duct paucity, pruritus, xanthomas, and cirrhosis that can lead to end-stage liver disease (approximately 95%).
Cardiac defects: peripheral pulmonic stenosis, tetralogy of Fallot, ventricular septal defect, atrial septal defect, aortic stenosis, and coarctation of the aorta (approximately 90%).
Dysmorphic face: prominent and broad forehead, deep-set eyes, prominent ears, triangular face with pointed chin, and broad nasal bridge (approximately 90%).
Renal abnormalities: dysplastic kidneys, glomerular mesangiolipidosis, and renal tubular acidosis (74%).
Skeletal malformations: butterfly vertebrae, hemivertebrae, and pathologic fractures of the long bones (70%).
Vascular abnormalities: cerebral artery stenosis and aneurysms, Moya-moya syndrome, reno-vascular abnormalities, and middle aortic syndrome (up to 15%) Ophthalmologic manifestations: ocular xanthelasma and posterior embryotoxon (78% to 90%).

Other features associated with ALGS include failure to thrive, short stature, immunodeficiency with recurrent infections, pancreatic insufficiency, delayed puberty, and developmental delays. The clinical presentation of ALGS is extremely variable and even patients from the same family with the same genetic mutation may have different presentations. Approximately 95% of patients with ALGS present with chronic cholestasis, usually within the first 3 months of life. Laboratory evaluation of these patients revealed elevated serum bile acids, elevated liver function tests (LFTs), and conjugated hyperbilirubinemia. Associated symptoms include xanthomas, growth failure, and pruritus.

### Methods

### Study Design and Treatment

ASSERT-EXT is a Phase 3, multi-center, open-label extension study designed to evaluate the long-term safety and efficacy of 120 μg/kg/day odevixibat in relieving pruritus in patients with ALGS. Patients who participated in the ASSERT trial (see Example 1) and met eligibility criteria for the ASSERT trial were eligible to participate. The duration of the treatment period is 72 weeks, followed by a 4-week Safety Follow-up Period. Clinic visits occur every 4 to 12 weeks. Patients who wish to continue receiving odevixibat after 72 weeks will have the opportunity to remain on treatment in an optional extension period with visits every 16 weeks until the drug is commercially available, provided continued use is supported by the risk-benefit profile, the patient has not been previously withdrawn or discontinued from the study, and the study is not terminated by the Sponsor. The 4-week Safety Follow-up Period will not occur for those who remain on treatment in the optional extension period.

Day 1 coincided with Week 24 in the ASSERT trial; however, if this was not possible due to logistic issues, patients enrolled in the ASSERT-EXT trial within 28 days of completion of the ASSERT trial. All patients in the ASSERT-EXT trial receive odevixibat 120 μg/kg/day. Two groups were analyzed up to an interim cut-off date of Sep. 9, 2022: treatment-naïve patients who received placebo in ASSERT (i.e., “placebo-odevixibat”) and patients who received odevixibat in ASSERT (i.e., “odevixibat-odevixibat”).

### Assessments

Outcomes evaluated include pruritus, serum BAs, sleep, and treatment-emergent adverse events (TEAEs). The Primary Efficacy Endpoint is the change from baseline in scratching through Week 72 as measured by the Albireo ObsRO caregiver instrument.

The secondary efficacy endpoints include: change in serum bile acid levels from baseline to Week 72; change from baseline through Week 72 in patient reported and observer reported itching and scratching severity scores, respectively, for the morning and evening assessment; percentage of patients achieving a clinically meaningful decrease in pruritus (pruritus responders) at each visit as measured by the Albireo ObsRO/patient reported outcomes (PRO) instruments; change from baseline to Week 72 in sleep parameters as measured with the Albireo ObsRO/PRO instruments (e.g. tiredness and number of awakenings); change from baseline to Week 72 in Pediatric Quality of Life Inventory (PedsQL) scores; assessment of Global Symptom Relief from baseline to Weeks 4, 12, 24, 48, and 72 as measured by patient, caregiver, and clinician Global Impression of Symptoms (PGIS, CaGIS, CGIS) items; assessment of Global Symptom Relief as measured by patient, caregiver, and clinician Global Impression of Change (PGIC, CaGIC, CGIC) items at Weeks 4, 12, 24, 48, and 72; and change in serum bile acid levels from baseline through Week 72.

The exploratory efficacy endpoints include: change from baseline to Week 72 in xanthomatosis as assessed by the Clinician Xanthoma Scale; change from baseline to Week 72 in ALT, AST, gamma-glutamyl transferase, and total bilirubin concentrations; change from baseline in biochemical markers and measures of bile acid synthesis (autotaxin, p-C4), total cholesterol, and triglycerides; change in growth from baseline to Weeks 24, 48, and 72, defined as the linear growth deficit (height/length for age, weight for age, and body mass index [BMI]) compared to a standard growth curve (Z-score, standard deviation [SD] from P50); incidence of biliary diversion and/or liver transplantation; and change from baseline in stage of liver fibrosis as assessed by elastography (where available). The PRO in addition to the ObsRO was be assessed in patients ≥8 years old.

Itching, Scratching, and Sleep Score. Itching, observed scratching, and sleep disturbance were recorded twice each day via eDiary. Patients and/or caregivers were instructed to complete the eDiary every day in the morning after the patient wakes and in the evening just before the patient goes to sleep for the first 24 weeks and the last 28 days before all remaining visits to the clinic. When logistically feasible, the same parent or caregiver recorded in the eDiary, and changes to the parent or caregiver who is performing the recording should be minimized. If a new parent or caregiver begins to complete the Albireo ObsRO eDiary during the course of the trial, they were provided with training during a clinic visit before they begin to use the eDiary. The caregiver recording in the eDiary was recorded. For the optional extension period, daily eDiary entries will not be completed after Week 72.

The eDiary included Albireo ObsRO and PRO items. Patients <8 years of age at time of signing the ICF were not be asked to complete the Albireo PRO items; only the Albireo ObsRO eDiary was completed by caregivers of patients in this age group. Older patients, 8 to <18 years of age at time of signing the ICF, completed the Albireo PRO items and the caregiver completed the Albireo ObsRO items. The Albireo PRO items assessed severity of itch, aspects of sleep disturbance (morning diary only), and tiredness. For patients 8 to 12 years of age, the caregiver read the Albireo PRO items along with the child and record the child's response. A guide was provided to the caregivers that provides standardized explanations of the Albireo PRO items, in case the patient was confused or required clarification about the meaning of a question. The Albireo ObsRO items assessed severity of observed scratching, aspects of observed sleep disturbance (morning diary only), and signs of tiredness (evening diary only). The Albireo ObsRO and PRO scratching and itch severity items use 0 to 4 response scales, where each response is distinguished by a unique facial expression, verbal anchor, number, and color code.

A daily AM and PM score for the Albireo ObsRO scratching item was averaged from the 2 ratings for each day. The AM score captured nighttime symptoms and the PM score captured daytime symptoms.

Serum Bile Acids. Blood samples for analysis of fasting total serum bile acid were drawn at Study Day 1, Week 4 to 24 and will be to Week 72 or EOT, and for Safety Follow-up visits. Fasting serum bile acids are also drawn at all visits during the optional extension period. Patients fasted (water intake only is permissible) for at least 4 hours prior to the collection of samples for serum bile acid. Exceptions were made for infants, <12 months of age, if unable to fast for the full 4 hours. Any visit at which a bile acid sample result is unreportable, an additional unscheduled visit for a repeat sample collection may be scheduled. Samples were processed and transported to a central laboratory per instructions in the laboratory manual.

Quality of Life Questionnaire (PedsQL). Patients and caregivers were asked to complete a QoL questionnaire (PedsQL) at Study Day 1, Weeks 12, 24, 48, and 72 or EOT visits, and at all visits during the optional extension period.

Global Impression of Change and Global Impression of Symptom Measures. Patients (≥8 years of age), caregivers, and clinicians completed GIC (PGIC, CaGIC, and CGIC) and the GIS measures (PGIS, CaGIS, CGIS) at Study Day 1 (PGIS only), Week 4, Week 12, and Week 24 and will complete them at Week 48 and Week 72 or EOT visits, and at all visits during the optional extension period.

The GIC items assessed change in itch (patient version), scratching (caregiver and clinician versions), and sleep (all versions) since starting the study drug. The GIS items assessed itch (patient version), scratching (caregiver and clinician versions), and sleep (all versions) in the past week. Caregivers and clinicians completed the GIC and GIS for all patients; those patients ≥8 years of age completed the patient version.

Clinician Assessment of Xanthoma. Changes in xanthoma as assessed by the Clinician Xanthoma Scale were measured at Study Day 1, Weeks 12, and 24 and will be measured at Weeks 48 and 72 or EOT visits. The clinician's assessment of the participant's xanthomatosis was focused on the number of lesions present and the degree to which the participant's lesions interfere or limit his or her activities. The clinician xanthoma scale uses a 5-point scale, in which 0 represents no evidence of xanthomatosis, 1 represents fewer than 20 scattered individual lesions, 2 represents more than 20 lesions that do not interfere with or limit activities, 3 represents large numbers of lesions that by their large numbers or size cause distortion of the face or extremities, and 4 represents xanthomas that interfere with function (such as hand use or ability to walk) because of excess size or number.

Biochemical Markers. Blood samples for analysis of clinical chemistry were drawn at Study Day 1, Week 4 to 24 and will be drawn at Week 25 to 72 or EOT, Safety Follow-up visits, and at all visits during the optional extension period. Total bilirubin, AST, ALT, and gamma glutamyl transferase assessments were included as part of the routine laboratory parameters. Blood samples for autotaxin and plasma-C4 will be drawn at Study Day 1, Weeks 12, 24, 48, and 72 or EOT, only for children with body weight >10 kg.

Growth. Growth was defined as the linear growth deficit (height/length for age, weight for age, and BMI) compared to a standard growth curve (Z-score, SD from P50), for patients age <18.

Liver transplant/Biliary Diversion Surgery. During the 72-week treatment period, if a patient permanently discontinues study drug prior to Week 72, additional telephone contacts will be made every 3 months after the EOT or Safety Follow-up (as applicable) to Week 72 to determine whether or not the patient has had biliary diversion or liver transplantation.

Safety Analysis. The primary safety analysis for this study included the incidence of total treatment emergent AEs (TEAEs) and TEAEs categorized by causality, severity, and seriousness assessments made by the investigator. Trends in safety were also evaluated for the following assessments: Physical examinations; Concomitant medications; Vital signs; Laboratory test results (including clinical chemistry, hematology, urinalysis, AFP, vitamins A, E, and 25-hydroxy vitamin D, and INR); Liver ultrasound and elastography (where available); and Discontinuations due to Aes.

Statistical Evaluation. The change from baseline to Week 72 in pruritus/scratching will be evaluated based on monthly score. The monthly (28 days) score will be calculated by taking average of 4 weekly scores within the month. The weekly score is the average of AM weekly score and PM weekly score. The pruritus/scratching scores for Baseline 1 and Baseline 2 will each be established from averaging 2 weekly scores within 14 days prior to treatment start on Study Day 1. Baseline 1 is defined as the last value prior to treatment start in the ASSERT study. Baseline 2 is defined as the last value prior to treatment start in ASSERT-EXT study. Baseline 2 will be used in all analyses unless otherwise specified.

All secondary efficacy variables will be analyzed descriptively for categorical and continuous data, as applicable. For continuous data, the change from baseline will be analyzed in addition to the actual visit values. For categorical data, frequency and percentage will be presented as appropriate. Change in growth (Z-score, SD from P50 standard growth curve) from Baseline 1 and other secondary endpoints will be analyzed by using one-sample T-test or Wilcoxon signed rank test as appropriate. The same method will be used to analyze change from Baseline 2. Change in growth (height/length for age, weight for age, and BMI) will also be displayed using graphical presentations.

Safety data from the full analysis set will be analyzed using descriptive statistics and summaries of SAEs, Aes, vital signs, clinical laboratory tests (hematology, clinical chemistry, and urinalysis), and concomitant medication.

### Results

At 24 weeks, the placebo-controlled, Phase 3 ASSERT-EXT study, odevixibat significantly improved pruritus and sleep disturbance and reduced serum bile acids (sBAs) in patients with ALGS. At the 24 week interim cut-off date, 49 patients had been treated in ASSERT-EXT (placebo-odevixibat: n=17; odevixibat-odevixibat: n=32). Patient demographics and baseline characteristics are summarized in Table 4.

TABLE 4
Demographics and Baseline Characteristics

| | | Odevixibat- |
|---|---|---|
| | Placebo-Odevixibat | Odevixibat |
| Parameter | n = 17 | n = 32 |
| Age, mean (SD), years | 5.9 (4.4) | 6.7 (3.5) |
| Male, n (%) | 6 (35) | 19 (59) |
| Genetic testing, n (%) | | |
| Mutation in JAG1 | 16 (94) | 29 (91) |
| Mutation in NOTCH2 | 1 (6) | 3 (9) |
| Scratching score, mean (SD) | 2.2 (1.0) | 1.1 (0.9) |
| Bile acids, mean (SD), μmol/L | 265 (160) | 155 (107) |
| ALT, mean (SD), U/L | 146 (66) | 239 (111) |
| AST, mean (SD), U/L | 151 (66) | 210 (115) |
| Total bilirubin, mean (SD), | 64 (60) | 57 (47) |
| μmol/L | | |
| Use of UDCA, n (%) | 16 (94) | 27 (84) |
| Use of anti-pruritus medication, n | 17 (100) | 31 (97) |
| (%) | | |

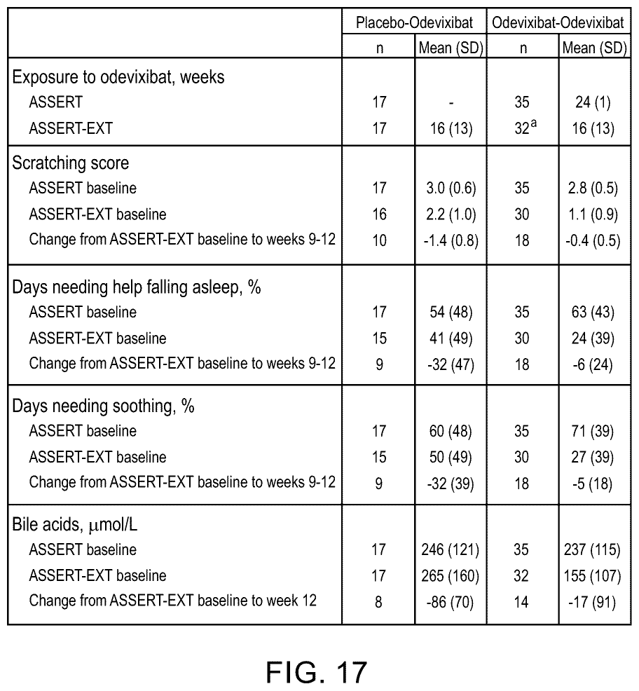

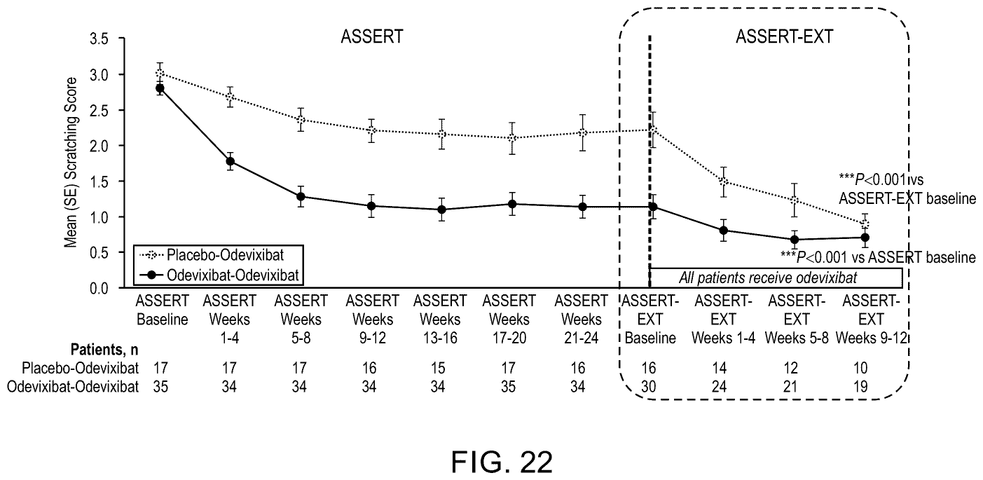

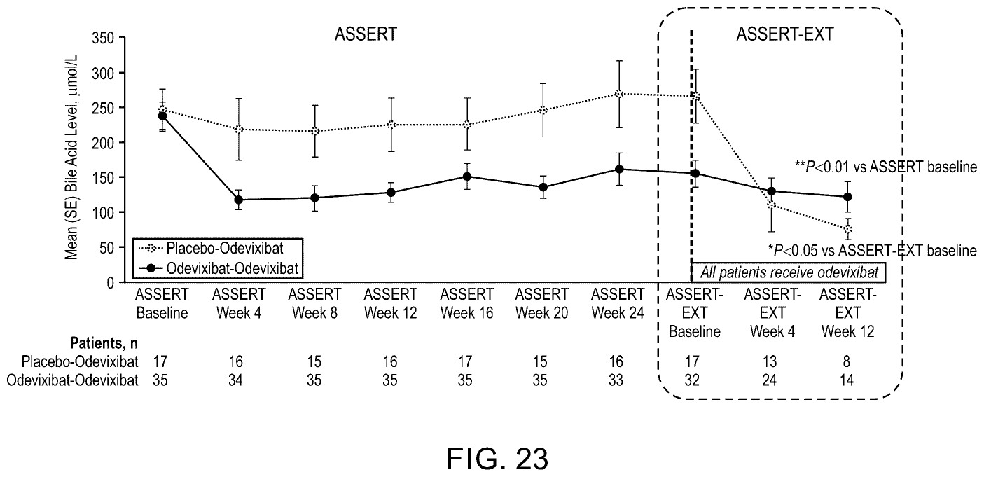

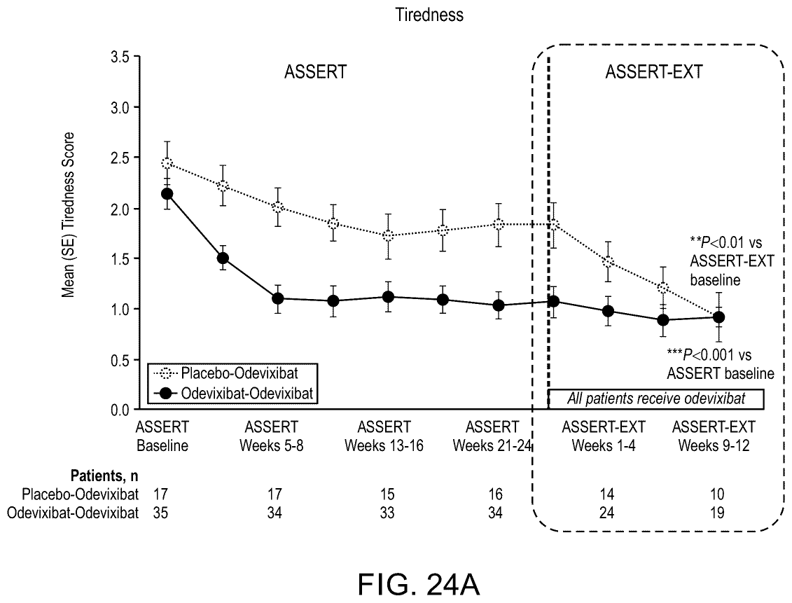

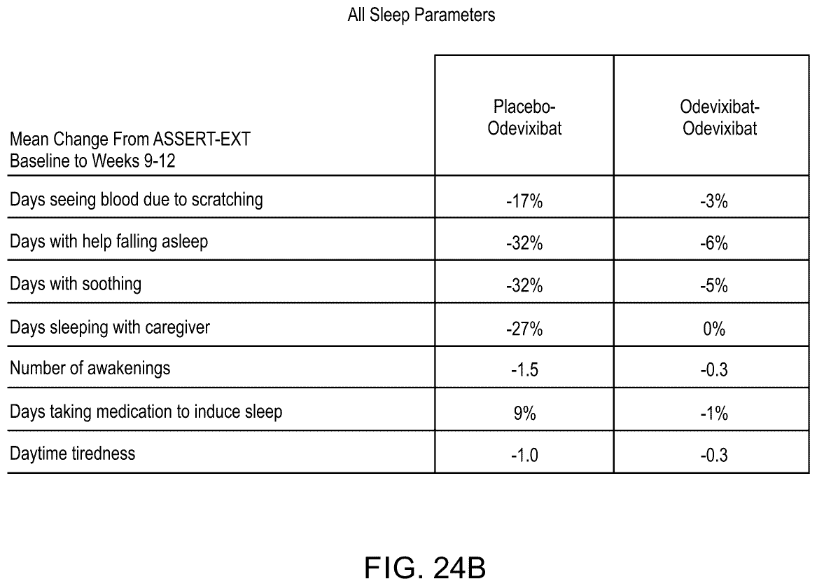

Patients in the odevixibat-odevixibat group entered ASSERT-EXT with improved pruritus, BAs, and sleep; these parameters continued to improve with longer treatment (FIG. 17). Patients in the placebo-odevixibat group had mean reductions in pruritus and BAs (FIG. 17); Mean reductions in percentage of days needing help falling asleep or soothing were reported in both groups (FIG. 17). Improvements in pruritus were sustained in the odevixibat-odevixibat group, and patients in the placebo-odevixibat group showed rapid improvements in pruritus after initiating odevixibat (FIG. 22). Reductions in bile acids were sustained in the odevixibat-odevixibat group, and patients in the placebo-odevixibat group showed rapid decreases in bile acids after initiating odevixibat (FIG. 23). Improvements in sleep parameters were sustained in the odevixibat-odevixibat group, and patients in the placebo-odevixibat group showed improvements in sleep parameters after initiating odevixibat (FIGS. 24A and 24B).

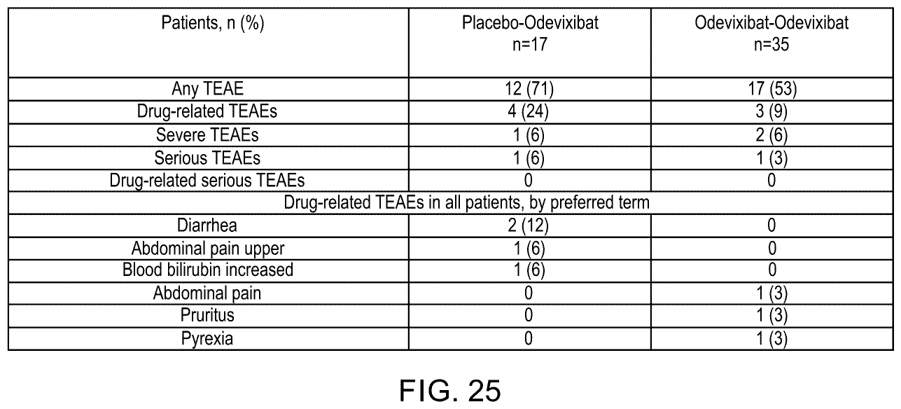

Most TEAEs were mild to moderate in severity; serious TEAEs were reported in 2/49 patients (4%). Incidence of treatment-emergent and drug-related diarrhea was 16% and 4%. No drug-related serious TEAEs or TEAEs leading to discontinuation or dose reduction were reported. See FIG. 25 for a summary of TEAEs. Scratching score and sleep parameters were based on the PRUCISION™ ObsRO instrument. Scratching score ranges from 0-4, with higher scores indicating worse symptoms.

Consistent with results from ASSERT, an interim analysis of ASSERT-EXT data showed that treatment-naïve patients with ALGS had rapid improvements in pruritus, sleep, and BAs with odevixibat; patients previously treated with odevixibat showed further improvements in these outcomes with continued treatment. Odevixibat treatment for ≥24 weeks in patients with ALGS was well tolerated.

### Example 3. Efficacy and Safety Outcomes with Odevixibat Treatment: Pooled Data from the Phase 3 ASSERT and ASSERT-EXT Studies in Patients with ALGS

Clinical features associated with cholestasis in Alagille syndrome (ALGS) may include accumulation of bile acids and other biliary components in the liver with spill-over into the systemic circulation, elevated hepatic laboratory parameters, and severe pruritus that can impair sleep. In the phase 3 ASSERT and ASSERT-EXT trials, odevixibat, an ileal bile acid transporter inhibitor, improved pruritus and sleep parameters and reduced bile acids (BAs) in patients with ALGS. Using pooled data from these studies, the efficacy and safety outcomes with odevixibat in patients with ALGS are described.

### Methods

In the completed 24-week ASSERT study, patients with ALGS with history of significant pruritus and elevated serum BAs were randomized 2:1 to once-daily odevixibat 120 μg/kg or placebo, respectively. Patients who completed ASSERT could enter ASSERT-EXT, an ongoing, 72-week open-label extension study in which all patients receive odevixibat 120 μg/kg/day. This pooled analysis included all odevixibat-treated patients from ASSERT and/or ASSERT-EXT and spans from patients' first dose of odevixibat to a data cut-off of Sep. 9, 2022. Assessments over up to 36 weeks of treatment included change in observer-reported scratching scores and sleep parameters, as well as levels of serum BAs, autotaxin (a proposed meditator of pruritus), and 7 alpha-hydroxy-4-cholesten-3-one (C4; a marker of bile acid synthesis). Safety evaluations included assessment of alanine aminotransferase (ALT), total bilirubin, and treatment-emergent adverse events (TEAEs).

### Results

At the data cut-off, 52 patients (mean age, 6.5 years; 48% female, Table 5) comprised the pooled population (median [range] odevixibat exposure, 30 [0.7-74] weeks). Seventeen of these patients were previously randomized to placebo in ASSERT. As of the data cutoff date, median (range) exposure to odevixibat was 30 weeks (0.7 to 74 weeks).

TABLE 5
Patient Demographics and Baseline^{a} Characteristics in
the Pooled Population of Odevixibat-Treated Patients.

| | | Pooled Odevixibatb |
|---|---|---|
| | Placebo^{a} | 120 μg/kg/day |
| | n = 17 | n = 52 |
| Age, mean (SD), years | 5.4 (4.4) | 6.5 (4.0) |
| Female, n (%) | 11 (65) | 25 (48) |
| Genetic testing, n (%) | | |
| Mutation in JAG1 | 16 (94) | 48 (92) |
| Mutation in NOTCH2 | 1 (6) | 4 (8) |
| Pruritus score, mean (SD) | 3.0 (0.6) | 2.6 (0.7)^{c} |
| Bile acids, mean (SD), μmol/L | 246 (121) | 246 (130) |
| Height Z score, mean (SD) | −1.4 (2.0) | −1.7 (1.4) |
| Weight Z score, mean (SD) | −1.5 (1.8) | −1.7 (1.2) |
| ALT, mean (SD), U/L | 149 (84) | 173 (79) |
| AST, mean (SD), U/L | 161 (91) | 164 (76) |
| Total bilirubin, mean (SD), mg/dL | 3.6 (3.3) | 3.3 (2.9) |
| Total cholesterol, mean (SD), mg/dL | 356 (184) | 336 (185) |
| Use of UDCA, n (%) | 16 (94) | 46 (89) |
| Use of antipruritus medication, n (%) | 17 (100) | 51 (98) |

Interim data cutoff date: Sep. 9, 2022.
^{a}Baseline is defined as the last assessment prior to start of placebo.
^{b}Baseline is defined as the last assessment prior to start of odevixibat.
^{c}n = 51.
ALT, alanine aminotransferase; AST, aspartate aminotransferase; UDCA, ursodeoxycholic acid.

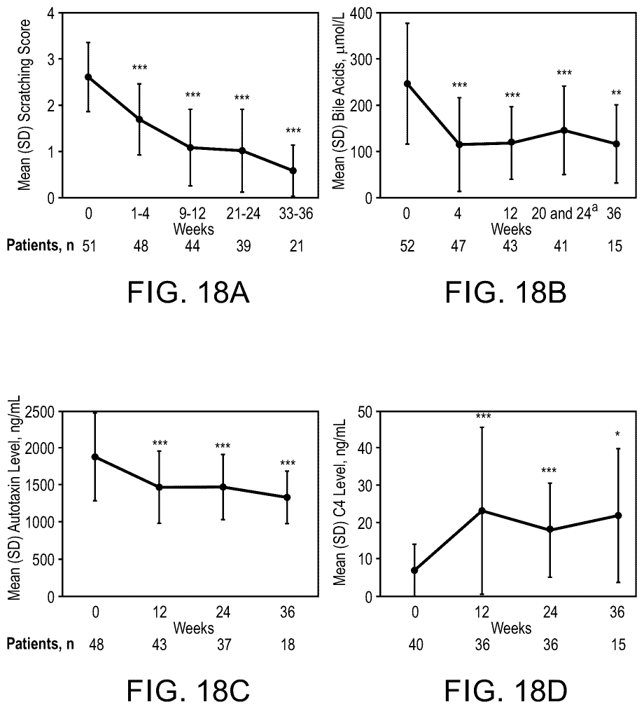

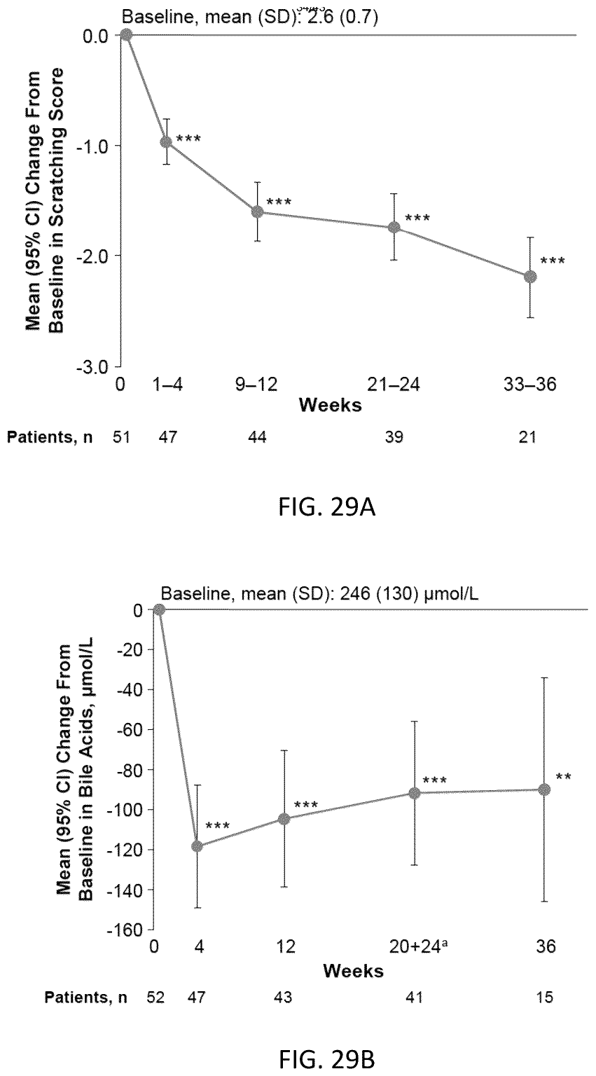

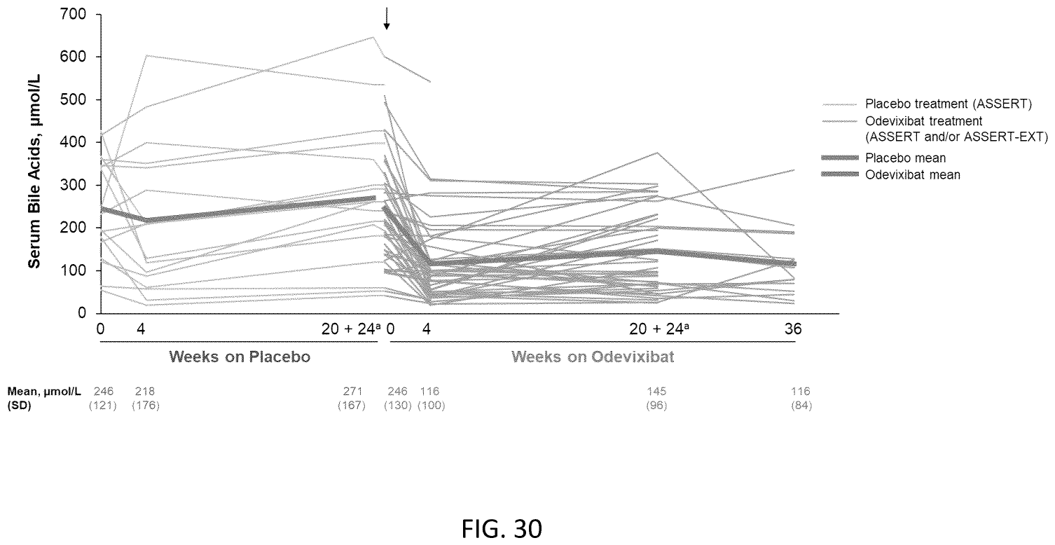

Compared with baseline, odevixibat treatment over up to 36 weeks resulted in rapid and significant mean improvements in pruritus and reductions in BA levels, as well as significant mean decreases in autotaxin and increases in C4 levels (FIGS. 18A-18D and see also FIGS. 29A-29B). Odevixibat-treated patients had reduced BA levels (mean values: baseline, 246 μmol/L; week 4, 116 μmol/L; average of weeks 20 and 24, 145 μmol/L; week 36, 116 μmol/L). In placebo-treated patients, mean BA values were 246 μmol/L at baseline and 271 μmol/L at the average of weeks 20 and 24 during the placebo treatment period. See FIGS. 18B and 29B. Serum bile acid levels over time in individual odevixibat-treated patients are shown in the FIG. 30.

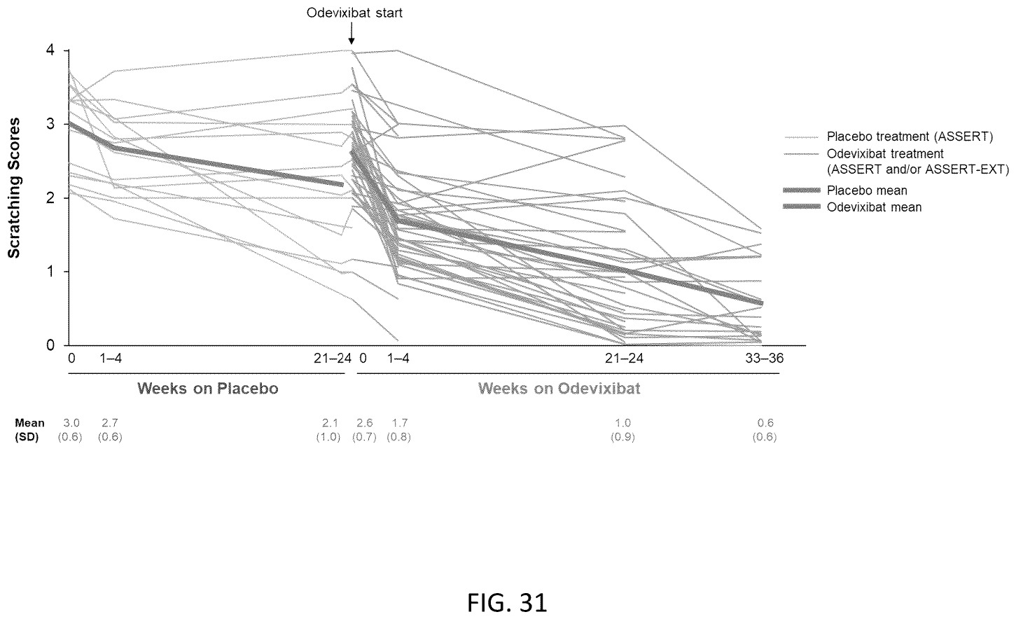

Odevixibat-treated patients had rapid reductions in scratching scores that were sustained over time: mean scores at baseline and at weeks 1-4, 21-24, and 33-36 were 2.6, 1.7, 1.0, and 0.6, respectively. In placebo-treated patients, mean scratching scores at baseline and weeks 21-24 during the placebo treatment period were 3.0 and 2.2, respectively. See FIGS. 18A and 29A. Scratching scores over time in individual odevixibat-treated patients are shown in the FIG. 31. At last assessment on odevixibat, 76% (n=37/49) of patients had a pruritus response (i.e., a ≥1 point reduction from baseline in scratching score). Mean p-C4 levels, in particular, approached normal levels following treatment with odevixibat. This effect suggests a return of bile acid homeostasis and improvement in cholestasis that could potentially predict better long-term outcomes. See FIGS. 18C and 18D.

There were significant changes from baseline to weeks 33-36 (n=21) in mean percentage of days seeing blood due to scratching, needing help falling asleep, needing soothing, and sleeping with caregiver (Table 6).

TABLE 6
Sleep Parameters With Odevixibat Treatment

| | | | Mean (95% CI) |
|---|---|---|---|
| | Mean (SD) % of Days | | Change From Baseline |
| | Baseline | Weeks 33-36 | to Week 33-36 |
| Patients, n | 50 | 21 | 21 |
| Days seeing | 35% (34%) | 5.7% (14%) | −30% (−42%, −17%)*** |
| blood due to | | | |
| scratching | | | |
| Days with help | 57% (45%) | 10% (18%) | −60% (−78%, −43%)*** |
| falling asleep | | | |
| Days with | 64% (43%) | 12% (25%) | −59% (−77%, −40%)*** |
| soothing | | | |
| Days sleeping | 43% (45%) | 10% (26%) | −44% (−64%, −24%)*** |
| with caregiver | | | |

P values are based on 1-sample t tests comparing the result at week 33-36 vs baseline.
***P ≤ 0.001.

Initial mean increases in ALT, AST, and GGT levels plateaued over time, while mean direct bilirubin and total bilirubin generally remained stable or improved; these changes were not indicative of long-term worsening of liver function. No patients had on-treatment elevations in ALT and total bilirubin that met Hy's law criteria. See also Example 3.

TEAEs were reported in 43 of 52 (83%) odevixibat-treated patients; most were mild to moderate (Table 7). In ASSERT, 71% of placebo-treated patients reported TEAEs. There were identical rates of severe adverse events (SAEs) in odevixibat and placebo groups.

Drug-related TEAEs were reported in 15 of 52 (29%) patients; the most common was diarrhea, which was reported in 6 of 52 (12%) patients. No patient experienced TEAEs that led to study discontinuation.

TABLE 7
TEAEs in the Pooled Population of Patients With
Alagille Syndrome Treated With Odevixibat.

| | | Pooled |
|---|---|---|
| | Placebo | Odevixibat |
| Patients, n (%) | n = 17 | n = 52 |
| Any TEAE | 12 (71) | 43 (83) |
| Drug-related TEAEs | 3 (18) | 15 (29) |
| Severe TEAEs | 2 (12) | 7 (14) |
| Serious TEAEs | 2 (12) | 6 (12) |
| Drug-related serious TEAEs | 0 (0) | 1 (2) |

Drug-related TEAEs, by preferred term
(occurring in >1 patient in the odevixibat group)

| | | |
|---|---|---|
| Diarrhea | 1 (6) | 6 (12) |
| Abdominal pain | 1 (6) | 2 (4) |
| Abdominal pain upper | 0 (0) | 2 (4) |
| Vomiting | 0 (0) | 2 (4) |

In patients with ALGS, odevixibat treatment for up to 36 weeks led to significant improvements in pruritus and sleep and significant reductions in BA levels. These changes occurred rapidly, with effects sustained over time. Consistent with effects on pruritus and BA levels, there were significant changes in autotaxin and C4 levels with odevixibat. Odevixibat treatment was generally well tolerated in patients with ALGS.

### Example 4. Odevixibat Treatment of Alagille Syndrome: A Case Report

ALGS, caused by mutations in JAG1 or NOTCH2, has variable presentation and may affect multiple organs, which can make diagnosis challenging (Singh S P, Pati G K. Euroasian J Hepatogastroenterol 2018; 8(2):140-47). Diagnostic criteria include cholestasis, cardiovascular defects, ocular posterior embryotoxon, atypical facial features, and/or skeletal malformation (Singh et al.). Patients with ALGS often suffer from impaired health-related quality of life (QoL) (Kamath B M, Baker A, Houwen R, et al. J Pediatr Gastroenterol Nutr 2018; 67(2):148-56), and persistent cholestasis may carry a particularly high disease burden. Cholestasis results in elevated serum bile acids (sBAs) and liver function tests, as well as symptoms like growth delays, xanthoma development, and severe pruritus, which can be particularly burdensome. Liver disease in ALGS can be progressive and may ultimately require liver transplantation.

Pathophysiologically, ALGS is characterized by bile duct paucity, which can result in accumulation of bile acids in the liver and subsequent spillover into circulation (Singh et al.; Karpen S J, Kelly D, Mack C, et al. Hepatol Int 2020; 14(5):677-89). While not fully understood, elevated sBAs have been proposed as a possible factor contributing to cholestatic pruritus (Karpen et al.). ALGS has historically been managed with supportive medical therapies like choleretic agents (e.g., ursodeoxycholic acid [UDCA]), other medications (e.g., rifampin), or surgical procedures like surgical biliary diversion (Singh et al.).

The ileal bile acid transporter (IBAT) is a regulator of the enterohepatic bile acid circulation (Karpen et al.). Given the central feature of cholestasis in ALGS and the role of IBAT in bile acid homeostasis, IBAT inhibitors may help alleviate symptoms of ALGS. The IBAT inhibitor odevixibat is indicated for treatment of pruritus in patients ≥3 months old with progressive familial intrahepatic cholestasis (PFIC), another pediatric cholestatic liver disease, in the US and for treatment of PFIC in patients aged ≥6 months in the European Union. Odevixibat has also been evaluated in 6 patients with ALGS in a phase 2 open-label study over 4 weeks; in that study, odevixibat reduced sBAs, itch, and sleep disturbance in a majority of those patients (Baumann U, Sturm E, Lacaille F, et al. Clin Res Hepatol Gastroenterol. 2021; 45(5): 101751). Here, the effects of odevixibat treatment over 8 months in a pediatric patient with severe pruritus and highly elevated sBAs with genetic confirmation of ALGS is described.

### Case Report

A male patient presented with prolonged jaundice after birth; subsequent blood work revealed hyperbilirubinemia, elevated liver enzymes (i.e., alanine aminotransferase [ALT], aspartate aminotransferase [AST], and gamma-glutamyl transferase [GGT]), and a normal total cholesterol level (105 mg/dL). The patient was referred to specialist care at 2 months of age. Biannual ultrasounds indicated splenomegaly and mild-to-moderate signs of portal hypertension. At 1 year of age, a liver biopsy was performed. The histology report suggested bile duct hypoplasia and mild fibrosis without additional details due to the very small tissue sample available for analysis. The patient had normal facial features, no skeletal abnormalities, and no overt cardiac symptoms; an initial diagnosis of PFIC was established in 2018. Genetic confirmation was not pursued then given that the patient's clinical features were consistent with PFIC and there was no effective treatment for familial cholestasis at the time; in addition, genetic testing was inaccessible in the surrounding area during this period (i.e., no genetic testing available at the treating hospital and long waiting times at external vendors).

Severe pruritus began at age 2: the patient's mother observed chronic scratching of the ears, arms, legs, and feet that resulted in bleeding, disturbed the child's sleep, and made it difficult for him to focus during the day. The child's pruritus also had a negative impact on the family's quality of life. Additionally, the patient had persistent abdominal pain and painful, irregular bowel movements. At age 3 years, the patient could only attend kindergarten for 4 hours per day.

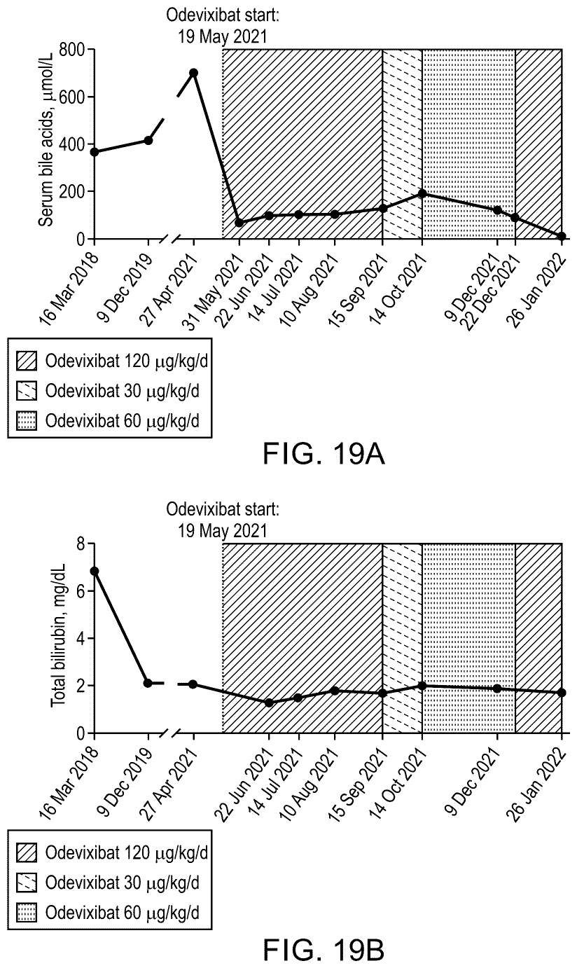

During this clinical course, the patient received UDCA (15 mg/kg body weight) and naltrexone (1 mg/kg/day), treatments that were pursued according to German treatment guidelines and following discussions among the care team and with the patient's parents. No significant clinical improvement was observed with these treatments; the patient consistently had elevated sBAs (FIG. 19A), bilirubin (FIG. 19B), liver enzymes, and ongoing pruritus. In April 2021, a compassionate-use program for odevixibat in patients with PFIC became available in Germany. Given the patient's pre-established clinical diagnosis and lack of response to other medical therapies, the patient started odevixibat 120 μg/kg/day as part of this program on May 19, 2021. The patient's sBAs (FIG. 19A) and total bilirubin (FIG. 19B) quickly decreased. The approximately 90% drop in sBAs was particularly dramatic, going from 700 μmol/L on Apr. 27, 2021, to 69 μmol/L on May 31, 2021. ALT, AST, and GGT levels rose in the months following odevixibat initiation (from 111 to 188 U/L, 116 to 140 U/L, and 187 to 487 U/L, respectively).

In August 2021, genetic testing was performed, and a heterozygous JAG1 mutation (c.2917-10A>G) was found. This variant has been previously reported by Stalke et al. as a variant of unknown significance in a patient with a characteristic ALGS phenotype (Stalke A, Skawran B, Auber B, et al. Clin. Genet. 2018; 93(3):665-70). In this report, genetic testing of the patient's parents found no evidence of the c.2917-10A>G variant in either the mother or the father, which suggests that the variant was a pathogenic de novo variant causing ALGS. Further diagnostic tests were undertaken at this time, and mild peripheral stenosis of the pulmonary artery and posterior embryotoxon were identified. The previous findings by Stalke et al., together with the patient data described here (i.e., genetic testing results and additional clinical findings), strongly suggest that the heterozygous c.2917-10A>G mutation is indeed pathogenic. Subsequently, the diagnosis of our patient was corrected to ALGS and the patient became ineligible for the compassionate-use program in PFIC. Odevixibat treatment was then continued off label. Additional testing in November 2021 revealed an elevated total cholesterol level (292 mg/dL), which gave further support to the revised diagnosis of ALGS in this patient.

To fulfill potential payor requirements, the patient's odevixibat dosage was changed multiple times, as follows: to 30 μg/kg/day from September to October 2021 (FIG. 19A), which was associated with an increase in sBAs, to 60 μg/kg/day from November to mid-December, and back to 120 μg/kg/day in mid-December, which corresponded with a drop in sBAs (FIG. 19A). sBA levels reached the normal range after 8 months of treatment (FIG. 19A).

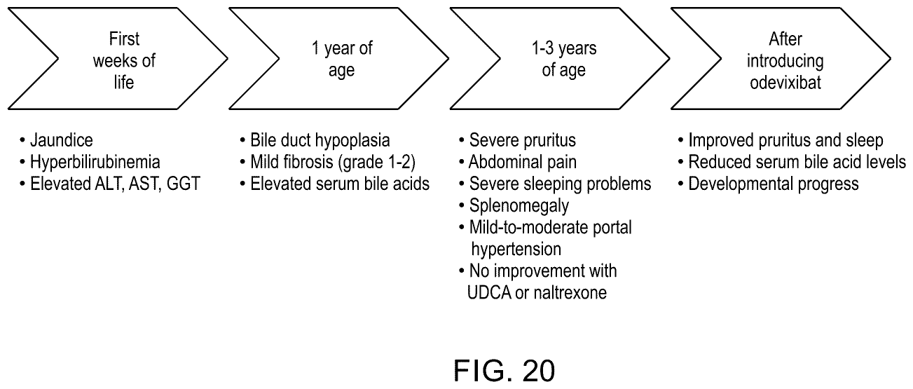

The mother reported that the patient started sleeping through the night within 4 days of starting odevixibat. Pruritus almost entirely disappeared within 2 weeks and stopped completely after 8 months. Furthermore, the patient's bowel movements improved after 1 week, and he no longer suffers from abdominal pain. The patient was able to start going to school, with improved focus, for at least 6 hours per day after starting odevixibat. Both the parents and daycare staff noted significant developmental progress. QoL was reported to significantly improve for the whole family. A flowchart of key milestones of disease progression and treatment success is shown in FIG. 20. The patient did not experience any adverse events with odevixibat.

### Discussion

In addition to supporting findings from the phase 2 trial of odevixibat in 6 patients with ALGS (Baumann et al.), this case report provides the first evidence of sustained treatment benefits and real-world effectiveness with odevixibat in ALGS. This case report also highlights the high burden of disease in patients with ALGS. Pruritus was one of the most bothersome symptoms of ALGS that greatly affected the patient's and his family's daily life. As exemplified by this patient, off-label medications that have been traditionally used to treat pruritus may not always work in children with ALGS. Neither UDCA nor naltrexone successfully treated symptoms in this case. The MAT inhibitor odevixibat was the first treatment that worked for this patient, with elimination of pruritus and rapid and robust reductions in sBAs. Importantly, odevixibat treatment also improved QoL for the patient and his family. Odevixibat was well tolerated in the patient described here, and treatment was associated with improved abdominal pain.

### Example 5. Changes in Hepatic Parameters with Odevixibat Treatment: Pooled Data from the Phase 3 ASSERT and ASSERT-EXT Studies in Patients with ALGS

Clinical features associated with cholestasis in Alagille syndrome (ALGS) may include accumulation of bile acids and other biliary components in the liver with spill-over into the systemic circulation, and elevated hepatic laboratory parameters. In the phase 3 ASSERT and ASSERT-EXT trials described in Examples 1 and 2 above, odevixibat improved pruritus and sleep parameters and reduced bile acids (BAs) in patients with ALGS. Using pooled data from these studies as described in Example 3, the changes in hepatic parameters with odevixibat in patients with ALGS are described in this example.

### Methods

In the completed 24-week ASSERT study, patients with a confirmed diagnosis of ALGS, history of significant pruritus, and elevated serum bile acids were randomized 2:1 to odevixibat 120 μg/kg/day or placebo, respectively. Patients who completed ASSERT could enter ASSERT-EXT, an ongoing 72-week extension study in which all patients received odevixibat 120 μg/kg/day. This pooled analysis spans from patients' first dose of odevixibat in ASSERT and/or ASSERT-EXT to a data cutoff at the end of the 72 week period; data from patients who received placebo in ASSERT are also presented for comparison. In both studies, laboratory assessments included measurement of serum alanine transaminase (ALT), Aspartate transaminase (AST), Gamma-glutamyltransferase (GGT), and total bilirubin levels. Caregivers rated patient pruritus using the PRUCISION™ instrument.

### Results

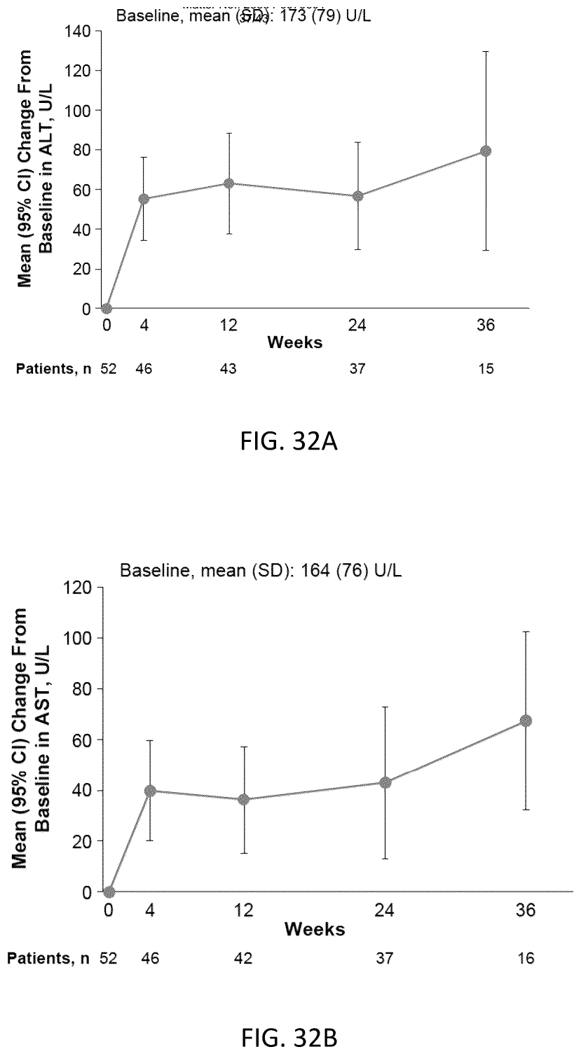

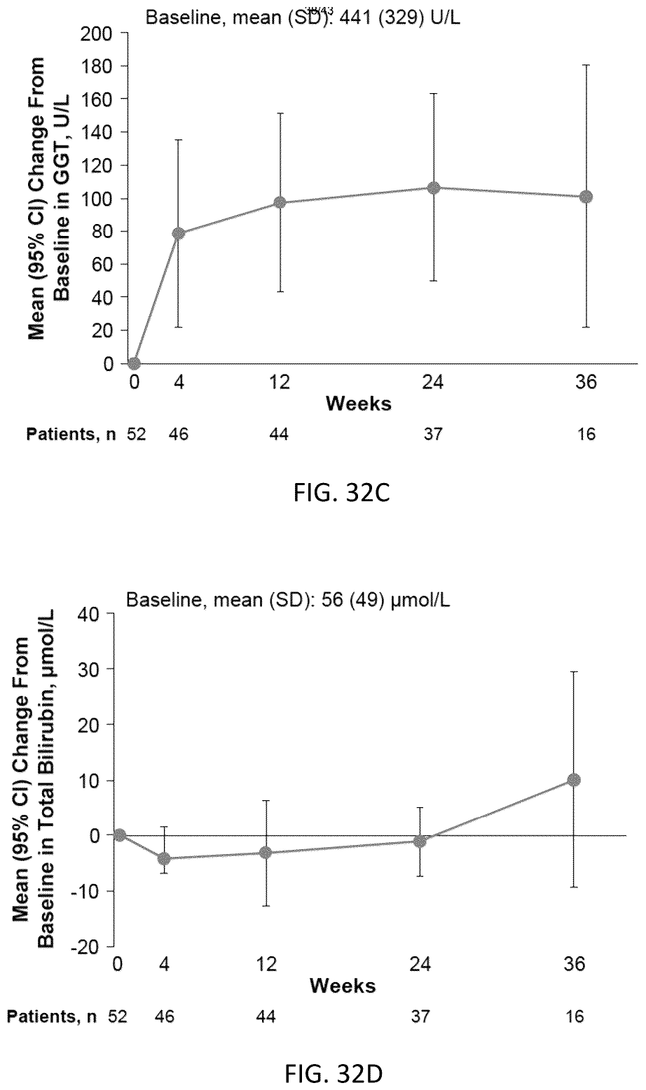

At the data cut-off, 52 odevixibat-treated patients (median [range] exposure: 30 [1-74] weeks) comprised the pooled population; 17 of these patients had initially received placebo in ASSERT (median [range] exposure: 24 [24-25] weeks). Mean elevations in hepatic parameters were observed at baseline in both groups (Table 8). In the odevixibat group, mean transaminase and GGT values generally increased from baseline to week 4 and plateaued or decreased from week 4 to week 48; mean changes from baseline in total bilirubin generally showed little change through week 48 (Table 8). In the placebo group, changes from baseline to week 24 in ALT, AST, and total bilirubin were minimal (Table 8). Potentially clinically significant elevations in AST and/or ALT to levels >3 times the baseline level (>3×BL) were observed in 4 odevixibat-treated patients, 1 of whom also had a peak ALT value >3×BL during treatment with placebo. In all 4 patients, concurrent total bilirubin levels were <2 times the baseline level. In addition, review of pertinent clinical information suggested drug-induced liver injury was unlikely, and all patients remained on treatment at the data cutoff. Overall, odevixibat-treated patients showed substantial improvements in pruritus regardless of increases from baseline in transaminases. See also FIGS. 32A-32D.

TABLE 8
Changes in Hepatic Parameters Over Time in Patients
With ALGS Treated With Odevixibat in ASSERT
and/or ASSERT-EXT or Placebo in ASSERT.

| | Pooled Odevixibat | | Placebo | |
|---|---|---|---|---|
| | n | Mean (SD) | n | Mean (SD) |
| ALT, U/L^{a} | | | | |
| Baseline | 52 | 173 (79) | 17 | 149 (84) |
| Change to week 4 | 46 | 55 (71) | 16 | 10 (76) |
| Change to week 12 | 43 | 63 (82) | 17 | 3 (39) |
| Change to week 24 | 37 | 57 (81) | 17 | −3 (48) |
| Change to week 36 | 15 | 79 (91) | NA | NA |
| Change to week 48 | 9 | 64 (102) | NA | NA |
| AST, U/L^{b} | | | | |
| Baseline | 52 | 164 (76) | 17 | 161 (91) |
| Change to week 4 | 46 | 40 (67) | 16 | −3 (71) |
| Change to week 12 | 42 | 36 (67) | 17 | −9 (40) |
| Change to week 24 | 37 | 43 (89) | 17 | −10 (43) |
| Change to week 36 | 16 | 67 (66) | NA | NA |
| Change to week 48 | 9 | 37 (72) | NA | NA |
| GGT, U/L^{c} | | | | |
| Baseline | 52 | 441 (329) | 17 | 536 (345) |
| Change to week 4 | 46 | 79 (191) | 16 | 52 (328) |
| Change to week 12 | 44 | 97 (178) | 17 | 88 (230) |
| Change to week 24 | 37 | 107 (170) | 17 | 59 (354) |
| Change to week 36 | 16 | 101 (149) | NA | NA |
| Change to week 48 | 9 | 2 (238) | NA | NA |
| Total bilirubin, μmol/L^{d} | | | | |
| Baseline | 52 | 56 (49) | 17 | 62 (57) |
| Change to week 4 | 46 | −4 (19) | 16 | −3 (12) |
| Change to week 12 | 44 | −3 (32) | 17 | 5 (20) |
| Change to week 24 | 37 | −1 (19) | 17 | 3 (15) |
| Change to week 36 | 16 | 10 (37) | NA | NA |
| Change to week 48 | 9 | −1 (16) | NA | NA |

^{a}Normal reference range varies by age and sex, but typical values are ≤50 U/L;
^{b}Normal reference range varies by age and sex, but typical values are ≤60 U/L;
^{c}Normal values for patients >6 months of age are ≤49 U/L;
^{d}Normal values for patients >1 month of age are ≤18.8 μmol/L.
ALGS, Alagille syndrome; ALT, alanine aminotransferase; AST, aspartate aminotransferase; GGT, gamma-glutamyl transferase; NA, not applicable.

As seen in Table 8, in the ASSERT and ASSERT-EXT studies, mean increases in serum transaminases without concurrent increases in total bilirubin were observed in odevixibat-treated patients with ALGS. Clinical data suggested these changes were not indicative of drug-induced liver injury and did not exclude the possibility of a beneficial treatment response.

### Example 6. Fat-soluble Vitamin Levels in Patients With Alagille Syndrome Treated With Odevixibat in the Phase 3 ASSERT Study

Alagille syndrome (ALGS) is a rare, genetic, multisystem disease that affects the liver and can lead to cholestasis, with resultant impairments in lipid absorption and subsequent fat-soluble vitamin (FSV) deficiency. In the 24-week ASSERT study described in Example 1, treatment with odevixibat resulted in significant improvements in pruritus and reductions in bile acids versus placebo in patients with ALGS. Effects related to FSVs in patients in ASSERT are summarized.

### Methods

Patients eligible for ASSERT included those of any age with ALGS, history of significant pruritus, and elevated serum bile acids; patients were randomized 2:1 to odevixibat 120 μg/kg/day or placebo, respectively. Vitamins A, D, E, and international normalized ratio (INR; surrogate for vitamin K) were measured throughout the study. Vitamins A, D, and E were measured at screening and randomization, week 12, and week 24. International normalized ratio (INR; surrogate for vitamin K) was measured at all visits. The following measures were determined: (i) percentage of patients with FSV insufficiency at baseline and week 24, and (ii) shifts from baseline to high or low post-baseline FSV values relative to normal ranges. Treatment-emergent adverse events (TEAEs) of FSV deficiency, TEAEs resulting from new or worsening FSV deficiency refractory to clinically recommended vitamin supplementation, and TEAEs represented any possible sequelae of FSV deficiency were evaluated.

### Results

As described in previous Examples, 52 patients (mean age, 6.3 years) received odevixibat (n=35) or placebo (n=17); all 52 patients completed the study. At baseline, mean levels of vitamins A, D, E, and INR were generally within normal range, reflective of vitamin supplementation received by a majority of patients. See Table 9.

TABLE 9
Patient Demographics and Clinical Characteristics.

| | | |
|---|---|---|
| Age, mean (SD), y | 5.4 (4.4) | 6.7 (3.8) |
| Female, n (%) | 11 (65) | 14 (40) |

Gene mutations, n (%)

| | | |
|---|---|---|
| JAG1 | 16 (94) | 32 (91) |
| NOTCH2 | 1 (6) | 3 (9) |

Prior vitamin supplementation, n (%)

| | | |
|---|---|---|
| Vitamin D and analogs | 11 (65) | 28 (80) |
| Vitamin K | 10 (59) | 18 (51) |
| Other plain vitamin preparations | 4 (24) | 14 (40) |
| Vitamin A, plain | 4 (24) | 6 (17) |
| Multivitamins, plain | 1 (6) | 4 (11) |
| Multivitamins with minerals | 4 (24) | 2 (6) |
| Vitamin A and D in combination | 1 (6) | 2 (6) |
| Combinations of vitamins | 3 (18) | 1 (3) |
| Vitamins, other combinations | 2 (12) | 1 (3) |
| Multivitamins, other combinations | 2 (12) | 0 |

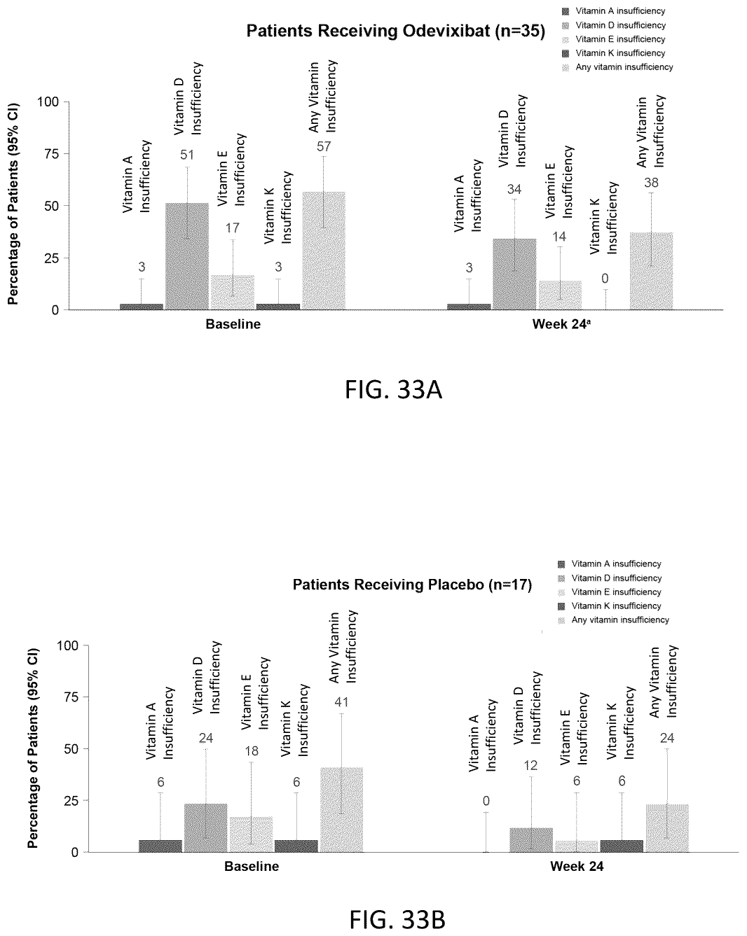

Overall, no clinically relevant changes were observed in mean FSV levels or INR in patients receiving odevixibat or placebo during the study. There were no significant differences between the odevixibat and placebo groups in proportions of patients with any vitamin deficiency at baseline (P=0.28) or at week 24 (P=0.32 [chi-squared tests]; FIGS. 33A and 33B). Within each treatment group, there were no significant differences in the proportions of patients with vitamin deficiency at baseline versus week 24 (odevixibat, P=0.11; placebo, P=0.18 [McNemar's tests]).

No patients shifted to low levels of vitamin A during the study. Shifts to low vitamin D levels were observed in 8/16 (50%) odevixibat-treated patients who had normal or high levels of vitamin D at baseline (none of these 8 patients were taking the protocol-recommended dosages of vitamin D supplementation at the time of the shift). There were no shifts to low levels of vitamin D in patients receiving placebo. There were no reports of pathologic bone fractures. See Table 10.

Shifts to low levels of vitamin E were observed in 4/29 (14%) odevixibat-treated patients who had normal or high levels of vitamin E at baseline (none of these four patients were taking vitamin E supplementation prior to randomization). There were no shifts to low levels of vitamin E in placebo-treated patients. Shifts from low or normal INR at baseline to high INR (i.e., low vitamin K) were observed in 12/30 (40%) patients receiving odevixibat (9 of whom were not taking the protocol-recommended vitamin K supplementation prior to randomization) and 3/14 (21%) patients who received placebo. See Table 10.

TABLE 10
Shift Analysis from Baseline to Highest
or Lowest Post-Baseline Values of FSVs.
Vitamin A, n/n (%)

| | | |
|---|---|---|
| Shifts to low^{a} | 0/16 | 0/34 |

Vitamin D, n/n (%)

| | | |
|---|---|---|
| Shifts to low^{a} | 0/13 | 8/16 (50) |

Vitamin E^{b}, n/n (%)

| | | |
|---|---|---|
| Shifts to low^{a} | 0/14 | 4/29 (14) |

INR^{c}, n/n (%)

| | | |
|---|---|---|
| Shifts to high^{d} | 3/14 (21) | 12/30 (40) |

^{a}Includes patients with normal or high baseline levels who shifted to low levels;
^{b}Reported as alpha tocopherol;
^{c}Surrogate measure for vitamin K;
^{d}Includes patients with normal or low baseline levels who shifted to high levels. Categories (low/normal/high) were defined based on the normal reference ranges of laboratory tests, including the lower limit of normal reference ranges in relevant age groups for vitamin A (0.2 mg/L), vitamin D (25-hydroxyvitamin D; 20 ng/L), vitamin E (alpha tocopherol; 2.0 mg/L), and the upper limit of normal reference range for vitamin K (INR; 1.1).
FSV, fat-soluble vitamin; INR, international normalized ratio.

Three patients (odevixibat, n=2; placebo, n=1) who shifted to high post-baseline INR had TEAEs of potential sequelae of vitamin K deficiency (Table 11). In two of these patients (odevixibat, n=1; placebo, n=1), TEAEs of INR increased were reported as potential sequelae of FSV deficiency on the same day as recorded shifts to high INR levels. The odevixibat-treated patient also experienced a TEAE of hematemesis on the same day as the shift to high post-baseline INR levels that responded rapidly to treatment with phytomenadione. In one additional odevixibat-treated patient, a TEAE of hematoma that occurred 2 days prior to the shift to high post-baseline INR levels was reported as a potential sequela of FSV deficiency; this TEAE was attributed to trauma.

Overall, TEAEs that were potential sequelae of FSV deficiency had a similar incidence in the odevixibat group (n=5 [14%]) and the placebo group (n=3 [18%]). See Table 11. The most common of these in the odevixibat group was hematoma, of which all incidences were attributed to trauma (Table 11). TEAEs of potential sequelae of FSV deficiency were reported in most patients as Grade 1 or 2 in intensity, were nonserious, were not related to the study drug, and did not lead to treatment interruption or study discontinuation.

In total, six patients reported TEAEs related to FSV levels (odevixibat, n=3 [9%]; placebo, n=3 [18%]; Table 11) One (3%) of 35 patients receiving odevixibat and 1 (6%) of 17 patients receiving placebo had an FSV deficiency TEAE refractory to clinically recommended vitamin supplementation. Both were TEAEs of vitamin D deficiency that were nonserious, resolved with modification of vitamin supplementation, did not lead to treatment interruption, and were assessed as unrelated to the study drug.

The four additional patients with TEAEs related to FSV levels had TEAEs that were not considered refractory to clinically recommended vitamin supplementation. Additional TEAEs related to FSV levels were increased INR (odevixibat, n=1; placebo, n=2) and decreased vitamin A and decreased vitamin E (odevixibat, n=1 each [both in the same patient]).

TABLE 11
TEAEs Related to FSV Deficiency and TEAEs
of Potential Sequelae of FSV Deficiency.

| | Placebo | Odevixibat |
|---|---|---|
| Patients, n (%) | (n = 17) | (n = 35) |
| Any FSV deficiency-related TEAE | 3 (18) | 3 (9)^{a} |
| Vitamin A decreased | 0 | 1 (3) |
| Vitamin D deficiency | 1 (6)^{b} | 1 (3)^{b} |
| Vitamin E decreased | 0 | 1 (3) |
| INR increased | 2 (12) | 1 (3) |
| Any potential sequelae events^{c} | 3 (18) | 5 (14) |
| Hematoma | 0 | 3 (9) |
| INR increased | 2 (12) | 1 (3) |
| Coagulopathy | 0 | 1 (3) |
| Hematemesis | 0 | 1 (3) |
| Contusion | 0 | 1 (3) |
| Epistaxis | 2 (12) | 0 |

^{a}One patient had both vitamin A and vitamin E decreased reported.
^{b}TEAEs considered to be refractory to clinically recommended vitamin supplementation; both were nonserious, did not lead to treatment interruption, and were assessed as unrelated to study drug.
^{c}Patients may have more than 1 TEAE of potential sequelae.
FSV, fat-soluble vitamin; INR, international normalized ratio; TEAE, treatment-emergent adverse event.

In the ASSERT study, proportions of patients with FSV insufficiencies were similar with odevixibat and placebo treatment. Shifts to lower levels of certain vitamins were numerically more common in patients treated with odevixibat compared with placebo; however, the rate of any FSV deficiency-related TEAE was lower in patients treated with odevixibat (9%) compared with placebo (18%). TEAEs of potential sequelae of FSV deficiency were reported at similar incidences in odevixibat- and placebo-treated patients. All patients completed the study.

### Example 7. Outcomes With Odevixibat Treatment in Patients With Alagille Syndrome: Analysis of Pruritus Responders From the Phase 3 ASSERT Study

The efficacy and safety outcomes in the subset of patients who met criteria for pruritus response following 24 weeks of odevixibat treatment in ASSERT are described.

### Methods

In ASSERT, patients with a confirmed diagnosis of ALGS, history of significant pruritus, and elevated serum bile acids were randomized 2:1 to odevixibat 120 μg/kg/day or placebo for 24 weeks. Treatment with concomitant medications for pruritus was permitted provided the patient was on a stable dosage for ≥4 weeks prior to enrollment that was maintained throughout the study.

Patient pruritus and daytime tiredness were rated twice daily by caregivers using the validated PRUCISION™ observer-reported outcome (ObsRO) (range: 0-4; higher scores indicate worse symptoms 5). Pruritus responders were defined as patients having a ≥1-point decrease in ObsRO scratching score from baseline to weeks 21-24). The following efficacy outcomes were evaluated in the subset of odevixibat-treated patients who met the prespecified criteria for pruritus response at the end of the study period:
Change from baseline to weeks 21-24 in ObsRO scratching score
Change from baseline to the average of weeks 20 and 24 in serum bile acid levels
Change from baseline to weeks 21-24 in ObsRO sleep parameters
Change from baseline to week 24 in caregiver-reported PedsQL total and domain scores
   Safety outcomes included treatment-emergent adverse events (TEAEs).

### Results

At baseline, patients entered the study with moderate-to-severe pruritus despite the use of antipruritic medications. Of the 35 patients who received odevixibat in ASSERT (median [range] exposure: 24 [21-26] weeks), 28 (80%) met criteria for pruritus response. Patient demographics and baseline disease characteristics are shown in Table 12.

TABLE 12
Demographics and Baseline Characteristics
in Odevixibat-Treated Pruritus Responders.

| | Pruritus Responders |
|---|---|
| | n = 28 |
| Age, mean (SD), years | 7.1 (3.8) |
| Male, n (%) | 20 (71) |

Genetic testing, n (%)

| | |
|---|---|
| Mutation in JAG1 | 25 (89) |
| Mutation in NOTCH2 | 3 (11) |
| Scratching score, mean (SD) | 2.8 (0.5) |
| Bile acids, mean (SD), μmol/L | 243 (120) |
| ALT, mean (SD), U/L | 185 (86) |
| AST, mean (SD), U/L | 174 (87) |
| GGT, mean (SD), U/L | 340 (219) |
| Total bilirubin, mean (SD), μmol/L | 45 (37) |
| Total bilirubin, mean (SD), mg/dL | 3 (2) |
| Use of UDCA, n (%) | 24 (86) |
| Use of antipruritus medication, n (%) | 28 (100) |

ALT, alanine aminotransferase; AST, aspartate aminotransferase; UDCA, ursodeoxycholic acid.

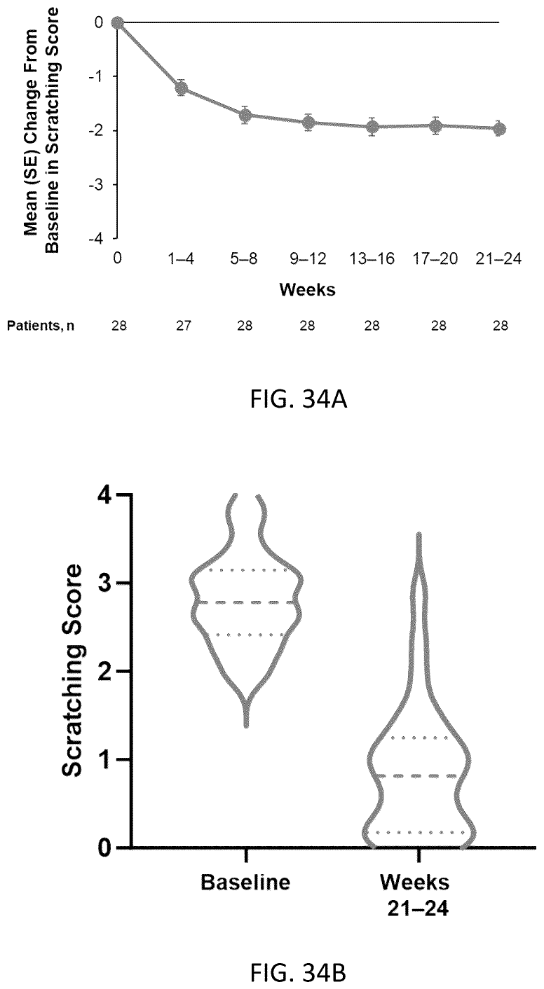

Odevixibat-treated patients who went on to achieve a pruritus response had a mean (SE) scratching score at ASSERT baseline of 2.8 (0.1). From baseline to weeks 21-24 of odevixibat treatment, pruritus responders had a mean (SE) change in scratching score of −2.0 (0.1) (FIGS. 34A and 34B).

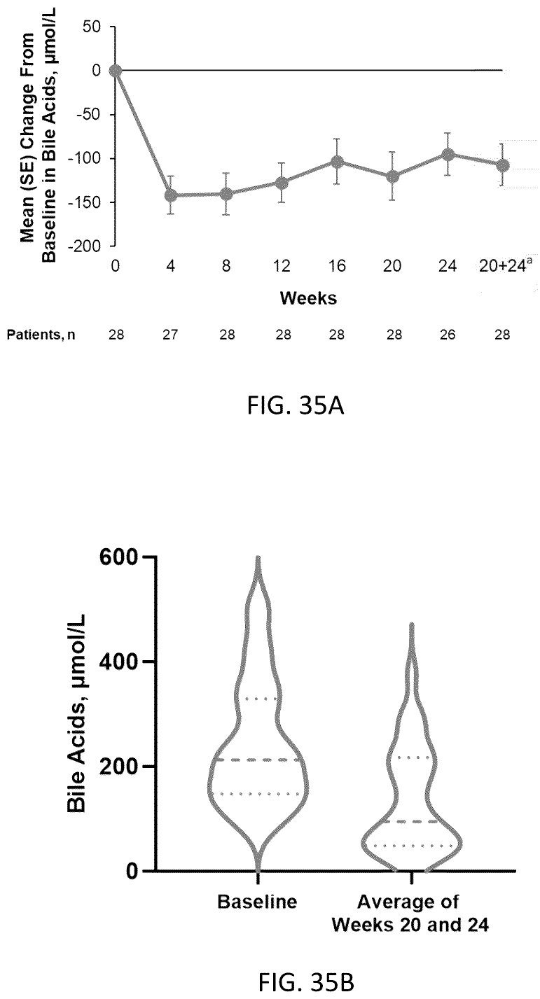

In odevixibat-treated patients who went on to achieve a pruritus response in ASSERT, mean [SE] bile acids at baseline were 243 μmol/L. The mean (SE) change from baseline to the average of weeks 20 and 24 in bile acids was −107 (23) μmol/L in pruritus responders (FIGS. 35A and 35B).

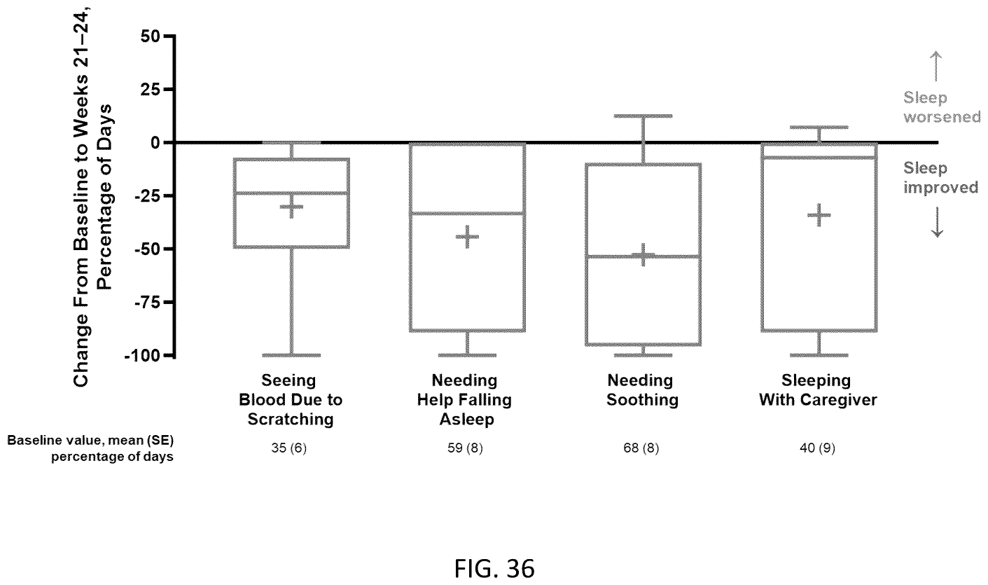

From baseline to weeks 21-24 of odevixibat treatment, mean and median reductions in the percentage of days seeing blood due to scratching, needing help falling asleep, needing soothing, and sleeping with caregivers were observed in pruritus responders (FIG. 36). Odevixibat-treated pruritus responders also had reductions in tiredness (mean [SE] change from baseline to weeks 21-24: −1.3 [0.2] on a scale of 0-4, with higher scores indicating worse symptoms) and number of awakenings (mean [SE] change from baseline to weeks 21-24: −2.2 [0.5]).

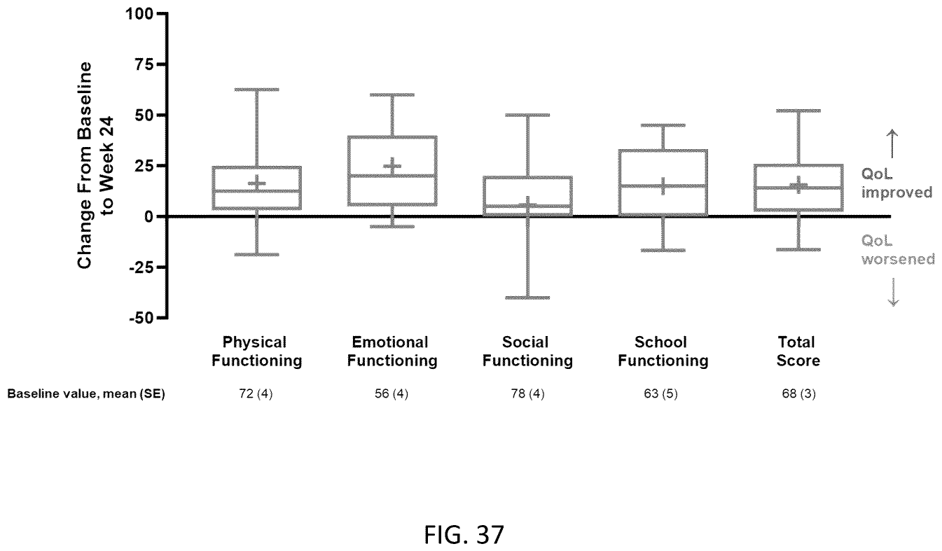

From baseline to week 24 of odevixibat treatment, pruritus responders experienced mean and median improvements in the caregiver-reported PedsQL total score, as well as the physical, emotional, social, and school functioning domain scores (FIG. 37).

Table 13 provides an additional summary of pruritus, bile acids, sleep, and quality of life in pruritus responders.

TABLE 13
Pruritus, Bile Acids, Sleep, and Quality of Life in Pruritus
Responders With Alagille Syndrome Treated With Odevixibat.

| | | | Week 24^{a} | | |
|---|---|---|---|---|---|
| | Baseline | | | | Mean (SE) |
| | | Mean | | Mean | Change From |
| | n | (SE) | n | (SE) | Baseline |
| Scratching score | 28 | 2.8 (0.1) | 28 | 0.9 (0.1) | −2.0 (0.1) |
| Bile acids, μmol/L | 28 | 243 (23) | 28 | 136 (19) | −107 (23) |
| Sleep parameters | | | | | |
| Days seeing blood due | 28 | 35 (6) | 27 | 5 (2) | −30 (5) |
| to scratching, % | | | | | |
| Days with help falling | 28 | 59 (8) | 27 | 15 (6) | −44 (8) |
| asleep, % | | | | | |
| Days with soothing, % | 28 | 68 (8) | 27 | 16 (5) | −53 (8) |
| Days sleeping with | 28 | 40 (9) | 27 | 8 (5) | −34 (8) |
| caregiver, % | | | | | |
| Daytime tiredness | 28 | 2.2 (0.2) | 28 | 1.0 (0.2) | −1.3 (0.2) |
| PedsQL total score^{b} | 24 | 68 (3) | 25 | 83 (2) | 16 (4)^{c} |

^{a}Scratching score and sleep parameters values are at weeks 21-24; bile acids values are at the average of weeks 20 and 24, and PedsQL scores are at week 24.
^{b}PedsQL total score range: 0-100 (higher scores indicate improved quality of life).
^{c}n = 23 patients.
PedsQL, Pediatric Quality of Life Inventory.

Overall, 21 (75%) pruritus responders reported any TEAE during treatment with odevixibat (Table 14). Most TEAEs were mild or moderate in severity; no TEAEs leading to death or study drug discontinuation were reported.

TABLE 14
Summary of TEAEs in Odevixibat-Treated Pruritus Responders.

| | Pruritus Responders |
|---|---|
| | n = 28 |
| Any TEAE, n (%) | 21 (75) |
| Mild | 10 (36) |
| Moderate | 8 (29) |
| Severe | 3 (11) |
| Drug-related TEAEs, n (%) | 8 (29) |
| Serious TEAEs, n (%) | 3 (11) |
| Drug-related serious TEAEs, n (%) | 1 (4)^{a} |
| TEAEs leading to treatment interruption, n (%) | 2 (7) |
| TEAEs leading to discontinuation, n (%) | 0 |

Drug-related TEAEs by preferred term, n (%)

| | |
|---|---|
| Diarrhea | 4 (14) |
| Vomiting | 2 (7) |
| Abdominal pain | 1 (4) |
| Abdominal pain upper | 1 (4) |
| Feces discolored | 1 (4) |
| Frequent bowel movements | 1 (4) |
| Hematemesis | 1 (4) |
| Nausea | 1 (4) |
| Blood triglycerides increased | 1 (4) |
| Hepatic enzyme increased | 1 (4) |
| INR increased | 1 (4) |
| Weight decreased | 1 (4) |

^{a}Patient had enteroviral gastroenteritis that led to vomiting and a Mallory-Weiss tear causing grade 3 serious TEAEs of hematemesis and increased INR assessed by the investigator as related to treatment; the TEAEs were responsive to vitamin K administration and resolved after 2 days with no changes to odevixibat dosage.
INR, international normalized ratio; TEAE, treatment-emergent adverse event.

These findings should be considered in light of general limitations associated with subjective measures such as those related to pruritus.

### DISCUSSION

In the ASSERT study, patients with ALGS who met criteria for pruritus response following treatment with odevixibat also experienced clinically meaningful reductions in bile acids and improvements in sleep and quality of life relative to baseline. Most TEAEs in odevixibat-treated pruritus responders were mild or moderate in severity, and none led to study drug discontinuation.

1.-53. (canceled)

54. A method for treating pruritus associated with Alagille Syndrome (ALGS) in a subject in need thereof, the method comprising orally administering to the subject a therapeutically effective amount of a pharmaceutical formulation comprising odevixibat, or a pharmaceutically acceptable salt thereof, wherein the subject exhibits a reduction in pruritus score relative to baseline following administration of the pharmaceutical formulation for at least 8 weeks.

55.-78. (canceled)

79. The method of claim 54, wherein the subject is a pediatric subject.

80. The method of claim 54, wherein the subject is administered about 20 μg/kg/day to about 120 μg/kg/day of odevixibat, or a pharmaceutically acceptable salt thereof.

81. (canceled)

82. The method of claim 54, wherein the pharmaceutical formulation of odevixibat, or a pharmaceutically acceptable salt thereof, comprises a plurality of particles, wherein each particle is between about 0.1 and about 1.5 mm in size and comprises odevixibat, or a pharmaceutically acceptable salt thereof, in an amount of from about 0.1% w/w to about 5.0% w/w based on the total weight of the particle.

83. The method of claim 82, wherein each particle comprises odevixibat, or a pharmaceutically acceptable salt thereof, in an amount of from about 0.5% w/w to about 2.0% w/w based on the total weight of the particle.

84.-100. (canceled)

101. The method of claim 54, wherein odevixibat is present as a sesquihydrate.

102. The method of claim 54, wherein odevixibat is present as a crystalline hydrate of odevixibat.

103. The method of claim 102, wherein odevixibat is present as crystal modification 1 of odevixibat.

104. The method of claim 103, wherein crystal modification 1 of odevixibat has an X-ray powder diffraction (XRPD) pattern, obtained with CuKα1-radiation, with at least specific peaks at ° 2θ positions 5.6±0.2, 6.7±0.2 and/or 12.1±0.2.

105.-106. (canceled)

107. The method of claim 54, wherein the reduction in pruritus score relative to baseline is about 1.5 to about 4.

108. The method of claim 54, wherein the reduction in pruritus score relative to baseline is about 1.5 to about 2.5.

109. The method of claim 54, wherein the reduction in pruritus score relative to baseline is about 2.

110. (canceled)

111. The method of claim 54, wherein the pruritus score at baseline is about 2.5 to about 4.0.

112.-114. (canceled)

115. The method of claim 54, wherein the subject exhibits a reduction in serum bile acid concentration relative to baseline.

116. (canceled)

117. The method of claim 115, wherein the reduction in serum bile acid concentration is about 50 μmol/L to about 180 μmol/L relative to baseline.

118.-120. (canceled)

121. The method of claim 115, wherein the serum bile acid concentration at baseline is about 180 μmol/L to about 600 μmol/L.

122.-123. (canceled)

124. The method of claim 54, wherein the subject exhibits an improvement in a liver parameter or biomarker relative to baseline following administration of the pharmaceutical formulation for at least 8 weeks.

125. The method of claim 124, wherein the liver parameter or biomarker is selected from the group consisting of autotaxin level, plasma C4 level, total bilirubin level, serum alanine aminotransferase (ALT) level, serum gamma-glutamyltransferase (GGT), and serum aspartate transaminase (AST) level.

126. The method of claim 54, wherein the subject exhibits a reduction in pruritus score relative to baseline following administration of the pharmaceutical formulation for about 24 weeks.

127. The method of claim 54, wherein the subject exhibits a reduction in pruritus score relative to baseline following administration of the pharmaceutical formulation for about 36 weeks.
